(19) United States
(12) Patent Application Publication
(10) Pub. No.: US 2005/0037445 A1
(43) Pub. Date: Feb. 17, 2005

(54) Oncology drug innovation

(76) Inventors: Hans Skovgaard Poulsen, Hellerup (DK); Nina Pedersen, Copenhagen (DK); Shila Mortensen, Gentofte (DK); Susanne Berg Sorensen, Hellerup (DK); Mikkel Wandahl Pedersen, Copenhagen (DK); Henrik Elsner, Broenshoej (DK)

Correspondence Address: BROWDY AND NEIMARK, P.L.L.C.;624 NINTH STREET, NW, SUITE 300, WASHINGTON, DC, 20001-5303, US

(21) Appl. No.: 10/482,029

(22) PCT Filed: Jun. 19, 2002
(86) PCT No.: PCT/IB02/03534

Related U.S. Application Data
(60) Provisional application No. 60/301,818, filed on Jul. 2, 2001.

(30) Foreign Application Priority Data
Jun. 25, 2001 (DK) PA 2001 00992

(51) Int. Cl. G01N 33/574

(52) U.S. Cl. 435/7.23

(57) ABSTRACT

The present invention describes methods for identification of molecules expressed at a different level on the cell surface of cancer cells compared to non-malignant cells and methods of identification of cancer specific promoters to be used singly or in combination for delivery and expression of therapeutic genes for treatment of cancer. The invention furthermore describes targeting complexes targeted to cell surface molecules identified by the methods of the invention. In embodiments of the invention said targeting complexes comprise the promoters identified by the methods of the invention. In addition the invention describes methods of identifying binding partners for the cell surface molecules and the binding partners per se. Methods of treatment using the targeting complexes and uses of the targeting complexes for the preparation of a medicament arc also disclosed by the invention. Furthermore, the invention describes uses of the cell surface molecules or fragments thereof for preparation of vaccines.

### FIELD OF INVENTION

The present invention relates to methods of identification of molecules on the cell surface of cancer cells and a method of identification of cancer specific promoters to be used singly or in combination for delivery and expression of therapeutic genes for treatment of cancer.

### BACKGROUND OF THE INVENTION

Approximately half of all patients with cancer have disseminated disease at the time of diagnosis. Existing cancer therapies are able to cure only 5-7% of these patients. Consequently, there is a great need for more effective drugs, which can be administered systemically alone or in combination with existing treatments. Methods utilising gene therapy to deliver efficient and specific treatment of cancer cells is therefore a promising strategy. However, strategies applied to this date have only had limited success and the development of suitable delivery systems need further development.

Delivery Vectors

The choice of the delivery vector for gene therapy is a major issue. Many vector systems have been tested for their suitability for gene transfer, including viral vectors such as retrovirus, adenovirus, adeno-associated virus, lentivirus and non viral vectors such as complexing with liposomes, cationic lipids or polycations. However, all of these vectors have specific advantages and limitations. Retrovirus requires mitotic division for transduction, but mediate long term expression, as they integrate in the genome. Adenovirus will transduce both dividing and non-dividing cells, but only transiently as they remain episomal. Adenovirus, however, are highly immunogenic and retrovirus are rapidly inactivated by the human complement system. Lentivirus does not induce immune response, but involve specific safety concerns; as it is a member of the immunodeficiency virus. More than 75% of all protocols so far have used viral vectors despite these are difficult and expensive to produce, there is a limited insert size of the therapeutic gene and there are many safety considerations to be made. Therefore, the majority of the protocols used for adenoviral vectors have administered the therapeutic gene by local delivery (injection into the tumour) to increase the local titer of the virus and avoid immunogenic response, but even the highest titer system has not yet been sufficient to cure local tumours. A major disadvantage of viral vector systems is that their uptake is unspecific and not targeted to the cancer cells. However, as adenovirus still is the preferred vector due to its efficiency of delivery, ways of reducing the immune response and target the virus to specific cells are under development. On the other hand, liposomes and polycation complexes, which are less immunogenic, easier to produce and do not need the safety considerations of viral vectors have much lower transfection efficiency than viral transduction and also lack the cell specificity. However, polycations have the ability to compact and neutralise the charge of the delivered DNA and PEI complexes appear relatively stable in the blood system (Goula et al., 1998; reviewed in Mountain, 2000).

To assure high specificity and to limit undesired side effects of the treatment, it is of importance to design a vector or vehicle, which targets and delivers the therapeutic gene in question to the cancer cells efficiently and with high specificity. However, as described below, this involves assembly of a multi component vector.

Receptor Targeting.

Functional receptors or other cell surface molecules, which can internalise by ligand or antibody binding on the cancer cell surfaces, can be used to target the gene delivery to the cells. Receptor targeted gene delivery by means of DNA conjugated to a ligand of the receptor offers a promising approach. The major advantages of targeted gene delivery are that receptor targeting can be performed without virus, thus eliminating many of the obstacles present in current strategies of gene therapy, Successful deliverance of genes to cancer cells using receptor targeting has been reported to a variety of different surface receptors including receptors for epidermal growth factor (Cristano and Roth, 1996, Frederiksen et al., 2000), folate (Gottschalk et al., 1994), transferrin (Wagneret al., 1990). High expression of a specific receptor is not always a pre-requisite for efficient receptor mediated uptake, as has been demonstrated for the epidermal growth factor receptor (Frederiksen et al., 2000). However, many of the receptors expressed by cancer cells are also expressed by normal cells to some extent, meaning that normal cells will often be targeted as well. This issue emphasises the need for further requirements for specificity for the expression or nature of the therapeutic gene.

Molecular Conjugates

For targeted gene therapy it is essential that the ligand to be internalised and DNA expressing the therapeutic gene are physically associated for receptor mediated uptake. Several methods have been used for preparing non-viral, synthetic vectors of targeted DNA molecular conjugates by associating cationic polymers, such as poly-L-lysine (Frederiksen et al., 2000) or polyethylenimine (PEI) (Kircheis et al., 1997) (polyplexes) with the ligand and DNA. Successful gene targeting has been reported for a number of molecular conjugates. The ligand has either been covalently linked to the polycation, or biotinylated ligand and polylysine were complexed via streptavidine to form condensed conjugates with DNA, which are internalised by the receptor of the ligand. One of the advantages of these system over virus mediated transfer is the lack of size limitation of the DNA. PEI complexes, in addition, appear to be able to pass the capillary barrier in lung, making this compound one agent for molecular conjugates.

Endosomal Release of Molecular Conjugate.

After endocytosis of the DNA/ligand conjugate by the receptor, the normal pathway would lead to degradation and loss of DNA. It has therefore proven essential to include an endosomolytic agent in the molecular conjugate. Adenovirus, replication deficient adenovirus and the viral capside have all proven to be very efficient for endosomal lysis, when included in the molecular conjugate. However, all the reservations of unspecific uptake, safety and immunogenic response applying to use of using adenovirus as vectors also apply for this system. Inclusion of other fusogenic peptides containing amino acid sequences from e.g. influenza virus, toxins or synthetic peptides in the molecular conjugate have been tested for cytoplasmic release. These have the advantage of less immunogenicity and lower cost, but have been shown to be less effective in endosomal lysis than adenovirus. However, if the molecular conjugate is formed using the polycationic PEI, inclusion of endosomolytic agents are not necessary, as PEI has an intrinsic endosome-buffering capacity resulting in endosomal swelling and rupture.

Cancer Specific Promoters

An increase in the specificity of the targeting of a therapeutic gene to cancer cells can be obtained if a tumour specific promoter controlling the expression can be used (reviewed in Nettelbeck et al., 2000). Promoters for genes, whose expression is specific for the malignant phenotype, but show no tissue specificity such as telomerase have been used. Also, promoters regulating oncofetal antigens, which are not normally expressed in the adult, have been found to be active in tumor cells, such as carcinoembryonic antigen (CEA). However, the activity of these promoters (compared to strong, constitutive active viral promoters) have often proven not to mediate sufficient expression of the therapeutic gene, wherefore the tumour specific genes have been used for activation of another, stronger promoter controlling the therapeutic gene. Another disadvantage of oncofetal promoters is that these promoters will only be active in a subset of tumour types, depending on the tissue origin of the tumour. Alternatively, synthetic promoters have been designed taking advantage of the fact that many oncogenes which are overexpressed in cancer cells are transcription factors, which can mediate high transcriptional activity from their respective DNA recognition sequences.

Therapeutic Genes

The product of a therapeutic gene must be able to effectively induce cell death. Gene therapy strategies for cancer treatment have used many different approaches. These include immunogene therapy such as cytokine stimulation of immune system (enhancing the immune response against tumour cells), selective prodrug activation, suicide genes, restoration of tumor suppressor genes and inhibition of activated oncogenes (reviewed in Frederiksen et al., 1999; Gunji et al., 2000). Indeed, most of the present therapeutic protocols in clinical trials against cancer involve immunotherapy. However, as the molecular phenotype of many types of cancer regarding aberrant expression or mutations of oncogenes and tumour suppressor genes, these are obvious candidates to target. Therapeutic gene products reducing expression or activity of oncogenes, such as antisense RNA or neutralising antibody fragments, have been tried and shown to inhibit proliferation. However, oncogene inactivation does not necessarily kill the cells and is therefore probably not applicable for short term treatment. One of the at present promising strategies is to reintroduce tumour suppressor genes, as most cancer cells exhibit loss of function of one or more of these genes. Of particular interest is the tumour suppressor gene TP53 encoding p53, which is a transcription factor, which activates genes known to be involved in cell cycle arrest and induction of apoptosis. Reintroduction of wild type p53 has been shown to markedly reduce tumour cell growth or induce apoptosis of cancer cells in both in vitro and in vivo systems (Roth et al., 1996; Nielsen and Maneval, 1998).

However, gene products rendering cells sensitive to otherwise harmless drugs has also been extensively used for gene therapy trials. In particular, the herpes simplex virus thymidine kinase (HSV-tk) in combination with the nucleoside analogue drug gangcyclovir has been used. However, the conversion of the drug to a toxic nucleo side analogue by the enzyme only will kill cells, which are dividing. However, the toxic products are transmitted to surrounding cells by the so-called “by-stander” effect, making the approach potential for systems with low targeting efficiency.

### SUMMARY OF THE INVENTION

Accordingly, it is a first objective of the present invention to provide methods for identifying a plurality of cell surface molecules, which are expressed at a different level in malignant cells compared with normal cells, comprising the steps of:
i) Providing at least 3 malignant cell lines selected from the group consisting of cell lines mentioned in table 1
ii) Providing at least 3 total RNA samples derived from normal tissue selected from the group consisting of liver, heart, kidney, lung, adrenal gland, colon, pancreas, small intestine, spleen, skeletal muscle, trachea, prostate, placenta, salivary gland, testes, leucocytes, leucocytes, brain, adipose tissue, bladder, breast, cervix, esophagus, larynx, ovary, rectum, skin, spinal cord, stomach, thymus, thyroid and uterus.
iii) Comparing the expression of mRNA in the cell lines according to step i) and tissue samples according to step ii)
iv) Identifying nucleic acid sequences, wherein
   a) there is a difference between the amount of mRNA expressed in one or more cell lines according to i) and the amount of mRNA expressed in one or more tissues according to ii); and/or
   b) there is essentially no difference in the amount of mRNA expressed in at least two cell lines according to i); and/or
   c) there is essentially no difference in the amount of mRNA expressed in at least two tissue samples according to ii); and
v) Selecting among the nucleic acid sequences according to iv), nucleic acid sequences encoding for potential cell surface molecules.

It is a second objective of the present invention to provide methods of identifying first nucleic acid sequences, which are capable of directing expression of second nucleic acid sequences operably linked thereto, wherein the level of said expression is different in malignant cells compared with normal cells comprising the steps of:
i) Providing at least 3 malignant cell lines selected from the group consisting of cell lines mentioned in table 1
ii) Providing at least 3 RNA samples derived from normal tissue samples derived from the group consisting of liver, heart, kidney, lung, adrenal gland, colon, pancreas, small intestine, spleen, skeletal muscle, trachea, prostate, placenta, salivary gland, testes, leucocytes, brain, adipose tissue, bladder, breast, cervix, esophagus, larynx, ovary, rectum, skin, spinal cord, stomach, thymus, thyroid and uterus.
iii) Comparing the expression of mRNA in the cell lines according to i) and tissue samples according to ii)
iv) Identifying second nucleic acid sequences, wherein
   a) there is a difference between the amount of mRNA expressed in one or more cell lines according to i) and the amount of mRNA expressed in one or more tissues according to ii); and/or
   b) there is essentially no difference in the amount of mRNA expressed in at least two cell lines according to i); and/or
   c) there is essentially no difference in the amount of mRNA expressed in at least two tissue samples according to ii)
v) Identifying first nucleic acid sequences operably linked to the second nucleotide sequences identified in step iv)

It is a third objective of the present invention to provide uses of a pharmaceutically effective amount of the cell surface molecules identified according to the present invention for the preparation of a vaccine. Furthermore, the present invention provides uses of a pharmaceutically effective amount of a nucleic acid sequence encoding a cell surface molecule identified according to the methods of the present invention for the preparation of a vaccine. The present invention also provides uses of a pharmaceutically effective amount of a cell surface molecule and/or a nucleic acid sequence encoding such a cell surface molecule for the preparation of a vaccine, wherein said cell surface molecule preferably comprises or essentially consists of or for example is a cell surface molecule mentioned in table 2.

It is a fourth objective of the present invention to provide uses of a cell surface molecule identified according to the methods described by the present invention as a drug target, wherein said drug target is capable of binding a binding partner and internalising said binding partner into cells expressing said cell surface molecule. Furthermore, the present invention provides uses of a cell surface molecule which preferably comprises or essentially consists of or for example is a cell surface molecule mentioned in table 2 as drug target, wherein said drug target is capable of binding a binding partner and internalising said binding partner into cells expressing said cell surface molecule.

It is a fifth objective of the present invention to provide methods of identifying and/or preparing specific binding partners comprising the steps of
i) Providing a cell surface molecule identified by the methods described by the present invention
ii) Identifying and/or preparing binding partners capable of associating with said cell surface molecules

It is furthermore an objective of the present invention to provide methods of identifying and/or preparing specific binding partners comprising the steps of.
i) Providing a cell surface molecule which preferably comprises or essentially consists of or for example is Transferrin receptor; such as type II membrane protein cone: for example is HP10481; such as type II membrane protein clone: such as HP10390; for example is PG40; such as TRC8; for example is TR2-11; such as OA3 antigenic surface determinant; for example is integrin alpha 6, For example GPIIb; such as vitronectin receptor alpha subunit; for example is integrin alpha-7; such as integrin alpha E precursor; for example is integrin alpha 6B; such as integrin alpha 5 subunit; for example is integrin beta-5 subunit; such as integrin alpha-3 chain; for example is RYK; such as amyloid precursor protein-binding protein 1; for example is putative transmembrane GTPase; such as membrane cofactor protein; FOR EXAMPLE GLVR1; for example is Mr 110,000 antigen; for example is syndecan-1; such as putative seven transmembrane domain protein; for example is LCA-homolog/LAR protein; such as M6 antigen; for example is Me491/CD63 antigen; such as multispanning membrane protein; for example is DDR; such as autocrine motility factor receptor; for example is insulin receptor precursor; such as IGF1R, for example is insulin-like growth factor II receptor; such as SAS; for example is TAPA-1; such as MICB; for example is MHC class II HLA-DR7-associated glycoprotein beta-chain; such as HLA-DP; for example is bone small proteoglycan I biglycan; such as CAR; for example is MEA11; such as interferon-gamma receptor alpha chain; for example is Polymeric immunoglobulin receptor; such as metabotropic glutamate receptor type 4; for example is metabotropic glutamate receptor 8; such as CLPTM1; for example is MAGE-4b; such as MAGE5a; for example is MAGE-3; such as MAGE-1; for example is MAGE6; such as MAGE-9; for example is MAGE11; such as CD24; for example is CD59; such as CD44; for example is low density lipoprotein receptor; such as very low density lipoprotein receptor, for example is N-CAM; such as lamin B receptor homolog TM7SF2; for example is putative T1/ST2 receptor binding protein precursor; such as NTR2 receptor; for example is RAGE-4; such as HLA-G1; for example is MOAT-C; such as alpha 2 delta calcium channel subunit isoform I; for example is LFA-3; such as L1-CAM; for example is AVPR2; such as C1 p115 C1; for example is TE2; such as RbP; for example is HCF1; such as IRAK; for example is CD151; such as surface antigen; for example is MAG; such as GPR19; for example is pcta-1; such as PRAME; for example is vasopressin activated calcium mobilizing receptor-like protein; such as serotonin receptor 5-HT4B; for example is serotonin 1 D receptor (5-HT1D˜); such as CD9; for example is LDL receptor member LR3; such as DR6; for example is tumor necrosis factor receptor; such as HG38; for example is urokinase-type plasminogen receptor; such as FGF receptor; for example is nerve growth factor receptor; such as cystine/glutamate transporter; for example is CB1 cannabinoid receptor (CNR1); such as PSG; for example is PSG13′; such as CPE-receptor; for example, is CRH2R; such as OCI5; for example is TRAIL receptor 2; such as HNMP-1; for example is kidney alpha-2-adrenergic receptor; such as erythropoietin receptor; for example is chondroitin sulphate proteoglycan versican V1; for example is mGluR1beta; such as CD97; for example is L6; such as NY-ESO-1; for example is T-cell receptor alpha delta; such as ror1; for example is ror2; such as SSTR2; for example is VESPR; such as IgG Fc receptor; for example is glutamate receptor subunit GluRC; such as HEK2; for example is PVR; such as CEA; for example is CC-chemokine-binding receptor JAB61; such as HER2; for example is HER3; such as hypothetical protein FL22357 similar to Epidermal growth factor receptor-related protein; for example is putative endothelin receptor type B-like protein; such as GLVR2; for example is P2X4 purinoreceptor; such as FPRL1; for example is Atrial natriuretic peptide clearance receptor; for example is gastrin/CCK-B receptor; such as Neuromedin B receptor; for example is GFRA3; such as GRPR; for example is CDH1; such as CDH2; for example is TGFBR1; such as TGFBR2; for example is TGFBR3; such as precursor of epidermal growth factor receptor.
ii) Identifying and/or preparing binding partners capable of associating with said cell surface molecules.

A further objective of the present invention is to provide isolated and/or purified specific binding partners capable of associating with cell surface molecules, which are expressed at a different level in malignant cells compared with normal cells, identified by the methods provided by the present invention. The present invention also provides isolated and/or purified specific binding partners capable of associating with a cell surface molecule which preferably comprises or essentially consists of or for example is a cell surface molecule mentioned in table 2.

It is also an objective of the present invention to provide methods of identifying novel drug targets, comprising the steps of
i) Providing a binding partner as described in the present invention,
ii) Identifying potential drug targets capable of associating with said binding partner

It is yet another objective of the present invention to provide drug targets identified by the methods described by the present invention.

Furthermore, it is an objective of the present invention to provide targeting complexes comprising:
i) A binding partner as described by the present invention; and
ii) A bioreactive species
wherein the targeting complex is capable of binding a cell surface molecule identified according to the methods described by the present invention and capable of being internalised into cells bearing said cell surface molecule.

The present invention also provides uses of binding partners as describes by the invention for the preparation of targeting complexes according to the invention.

It is yet a further objective of the present invention to provide pharmaceutical compositions comprising of the targeting complexes described by the present invention together with a pharmaceutically acceptable carrier.

It is even a further objective of the present invention to provide methods of treatment of a premalignant and/or malignant conditions in an individual in need thereof, comprising administering to said individual a pharmaceutically effective amount of the targeting complexes described by the present invention.

Furthermore, it is an objective of the present invention to provide uses of the targeting complex described by the present invention for the preparation of a medicament for the treatment of a premalignant and/or malignant conditions in an individual in need thereof.

### LEGEND TO FIGURES

FIG. 1 illustrates the principle of targeted gene therapy.

FIG. 2 illustrates a comparison between gene expression measured by Chips analysis and RT-PCR. The figure shows a quality test of cDNA used for RT-PCR validation of Chips analysis by RT-PCR of Glyceraldehyde-3-phosphate dehydrogenase (GAPDH).

FIG. 3 illustrates a comparison between gene expression measured by Chips analysis and RT-PCR for Pro 221 (IA-1).

FIG. 4. illustrates a comparison between gene expression measured by Chips analysis and RT-PCR of Pro 30 (KIA0042).

FIG. 5. illustrates a comparison between gene expression measured by Chips analysis and RT-PCR of Pro 41 (MAD2).

FIG. 6. illustrates a comparison between gene expression measured by Chips analysis and RT-PCR of Pro 210 (lamin B1).

FIG. 7 illustrates a comparison between gene expression measured by Chips analysis and RT-PCR of Pro 71 (CDKN2A).

FIG. 8 illustrates a comparison between gene expression measured by Chips analysis and RT-PCR of cell surface molecule DR6.

FIG. 9. illustrates a comparison between gene expression measured by Chips analysis and RT-PCR of cell surface molecule LRP8.

FIG. 10. illustrates a comparison between gene expression measured by Chips analysis and RT-PCR of cell surface molecule NTPXR.,

FIG. 11. illustrates a comparison between gene expression measured by Chips analysis and RT-PCR of cell surface molecule NCAM1.

FIG. 12A illustrates a comparison between gene expression measured by Chips analysis and RT-PCR of cell surface molecule GluR2 (GRIA2).,

FIG. 12B illustrates a comparison between gene expression measured by Chips analysis and RT-PCR of cell surface molecule ITGAV.

FIG. 13 illustrates a comparison between gene expression measured by Chips analysis and western blotting of mGluR8.

FIG. 14. illustrates a comparison between gene expression measured by Chips analysis and western blot analysis for NPTXR.

FIG. 15. illustrates a comparison between gene expression measured by Chips analysis and western blot analysis for NCAM1.

FIG. 16. illustrates a comparison between gene expression measured by Chips analysis and western blot analysis for GluR2 (GRIA2).

FIG. 17. illustrates a comparison between gene expression measured by Chips analysis and western blot analysis for ITGAE.

### DETAILED DESCRIPTION OF THE INVENTION

Definitions

Binding partner: See “cell surface molecule binding partner”.

Bioreactive species: Any molecule, which can directly or indirectly exert a biological influence on a target cell.

Bp: Base Pair

Cell surface molecules: Molecules naturally associated with the cell surface.

Cell surface molecule binding partner: Any molecule that can associate specifically with a cell surface molecule. Throughout the text the terms “Cell surface molecule binding partner” and the shorter term “binding partner” are used interchangeably and both terms are equivalent to one another throughout the text.

Enhancer: Nucleic acid sequence, which can enhance the transcription of a second nucleic acid sequence operably linked thereto.

First nucleic acid sequences: Nucleic acid sequences, which are capable of directing expression of second nucleic acid sequences operably linked thereto.

Normal cells: Non-malignant cells that are of non-malignant origin.

Normal tissue: Non-malignant tissue

Promoter: First nucleic acid sequences, which are capable of directing expression of second nucleic acid sequences operably linked thereto.

Second nucleic acid sequences: Nucleic acid sequences, which are capable of being expressed, such as mRNA may be transcribed from such nucleic acid sequences, when they are operably linked to first nucleic acid sequences.

Silencer A nucleic acid sequence, which is capable of repressing the transcription of a second nucleic acid sequence operably linked thereto.

Targeting complex: Complex which comprises at least one binding partner and a bioreactive species and which is capable of be internalised into cells.

Embodiments of the Invention

It is becoming increasingly obvious, that if gene therapy of cancer is to become an effective, alternative or adjuvant treatment of cancer, in particular of disseminated disease, several requirements must be resolved. These include for example i) targeting of the complex to be efficient and cancer specific; ii) expression of the therapeutic gene to be efficient and cancer specific and iii) that the molecular conjugate is non-immunogenic, has a high stability after systemic administration and is able to cross capillary barriers.

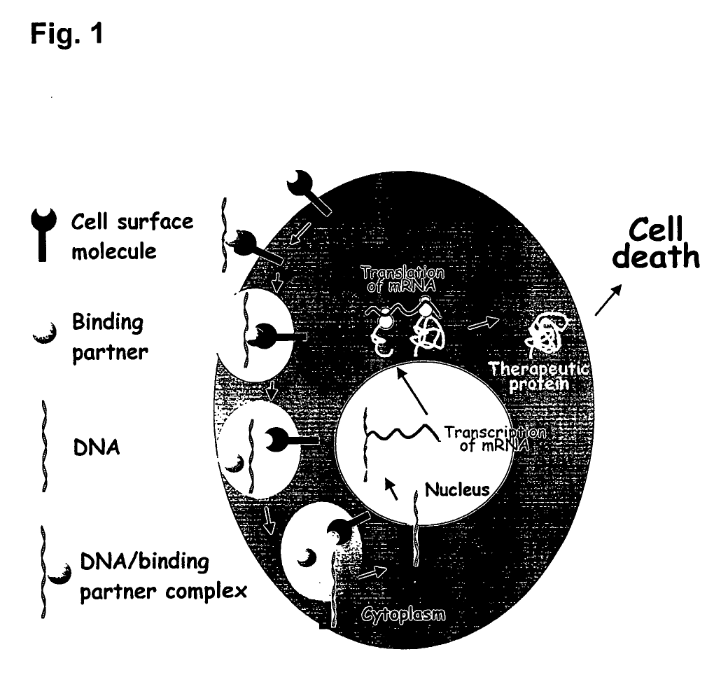

In one preferred embodiment, the present invention relates to the use of novel, high throughput screening methods for identification of genes specifically expressed by cancer cells and their application for double-targeted gene transfer and expression of therapeutic genes for treatment of cancer. An example of the principle of double-targeted gene transfer is outlined in FIG. 1. The screening methods according to the present invention enable the identification of novel molecules expressed by the cancer cells.

In one embodiment the method will be applied on identification of gene expression of suitable molecules expressed by small cell lung cancer (SCLC) cells. Small cell lung cancer is a highly aggressive neoplasm, comprising of approximately 25% of all lung cancer cases. The disease is almost always disseminated at the time of diagnosis. SCLC is treated with different chemotheraputic drugs alone or in combination with radiation therapy. Despite intensive attempts to improve treatment, and regardless of the fact that most patients respond well to the treatment in the beginning, the mortality rate is high. Existing cancer treatments are able to cure only 5-7% of these patients and the 5-year survival rate is extremely poor (5-15%). SCLC patients therefore are in great need of the development of new therapies.

The molecular phenotype of the disease has been thoroughly characterised and the aberrant expression of oncogenes (particularly of the myc-family) in addition to the loss of function of several tumour suppressor genes (such as p53 and Rb) have been found for more than 80% of SCLC tumours. These phenotypes are also found most cell lines deriving from SCLC tumours (reviewed in Frederiksen et al., 1999), allowing the cell lines to be used as an experimental tool for in vitro testing of potential anticancer drugs. In addition, these cell lines can be propagated in vivo in nude mice, thus allowing testing of developed drugs an in vivo situation. Therefore cell lines derived from SCLC will be used for the initial screening of gene expression for identification of cancer specific (or highly expressed) surface molecules and regions with particular transcriptional activity (promoters) in SCLC cells.

It is preferred that a variety of different SCLC cell lines established by different laboratories and from different patients are used with the present invention, in order to identify genes expressed in a large number of SCLCs. Furthemore, it is preferred that expression in these SCLC cell lines is compared with expression in a variety of normal tissues, which preferably is representative of different tissues of endodermal, ectodermal and mesodermal origin.

It is possible within the present invention to apply a biphasic strategy, however in certain embodiments of the present invention other strategies may be applied. A biphasic strategy according to the present invention may for example be a gene therapy drug that via systemic administration can target cancer cells effectively through binding to functional, transport-competent receptors on the surface and which subsequently allows expression of the gene effectively in the cancer cells by a promoter, which is specifically active or hyperactive in the cancer cells.

Amino Acids and Nucleic Acids

Throughout the description and claims either the one letter code or the three letter code for natural amino acids are used. Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form; or a sequence of mixed L forms and D forms.

Where nothing is specified it is to be understood that the C-terminal amino acid of a polypeptide of the invention exists as the free carboxylic acid, this may also be specified as “—OH”. The N-terminal amino acid of a polypeptide comprise a free amino-group, this may also be specified as “H—”.

Where nothing else is specified amino acid can be selected from any amino acid, whether naturally occurring or not, such as alpha amino acids, beta amino acids, and/or gamma amino acids. Accordingly, the group comprises but are not limited to: Ala, Val, Leu, lie, Pro, Phe, Trp, Met, Gly, Ser, Thr, Cys, Tyr, Asn, Gin, Asp, Glu, Lys, Arg, His, Aib, Nal, Sar, Orn, Lysine analogues DAP and DAPA.

The term “nucleic acid” is meant to encompass DNA and RNA as well as derivatives thereof such as peptide nucleic acids (PNA) or locked nucleic acids (LNA) throughout the description.

Methods to Identify Cell Surface Molecules and Promoters

The methods used to identify cell surface molecules and/or first nucleic acid sequences, which are capable of directing expression of second nucleic acid sequences operably linked thereto according to the present invention preferably involve the comparison of levels of mRNA found in malignant cell lines with the levels of mRNA found in normal tissues.

Preferably, the malignant cell lines according to the present invention are mammalian cell lines, more preferably human cell lines. Yet more preferably, the cell lines are derived from small cell lung carcinomas (SCLC). Even more preferably, the cell lines are selected from the group consisting of cell lines mentioned in table 1. More preferably, the cell lines are selected from the group consisting of CPH 54 A, CPH 54 B, GLC 2, GLC 3, GLC 14, GLC 16, GLC 19, GLC 26, GLC. 28, DMS853, DMS 79, DMS 92, DMS 114, DMS 153, DMS 273, DMS 406, DMS 456, NCI H69, NCI N417, MAR H24 and MAR 86 MI.

Yet even more preferably the cell lines are selected from the group consisting of CPH 54A, CPH 54 B, CHP 136A, GLC 2, GLC 3, GLC 14, GLC 16, GLC 19, GLC 26, GLC 28, DMS 53, DMS 79, DMS 92, DMS 114, DMS 153, DMS 273, DMS 406, DMS 456, NCI-H69, NCI-N417, MAR H24 and MAR 86MI.

Most preferably, the cell lines are selected from the group consisting of DMS 53, DMS 70, DMS 92, DMS 114, DMS 153, DMS 273, NCI 417 and NCI H69.

Preferred cell lines according to the present invention are listed in table 1 together with their accession numbers.

TABLE 1
Deposit Accession numbers of small cell lung cancer cell lines

| SCLC | Culture | (Provisional) | |
|---|---|---|---|
| cell line | Collection | Accession no. | Depositor |
| CPH 54A | ECACC | 01061905 | ODIN |
| CPH 54B | ECACC | 01061906 | Medical A/S |
| GLC 2 | ECACC | 01061907 | |
| GLC 3 | ECACC | 01061908 | |
| GLC 14 | ECACC | 01061909 | |
| GLC 16 | ECACC | 01061910 | |
| GLC 19 | ECACC | 01061911 | |
| GLC 26 | ECACC | 01061912 | |
| GLC 28 | ECACC | 01061913 | |
| DMS 406 | ECACC | 01061914 | |
| DMS 456 | ECACC | 01061915 | |
| MAR H 24 | ECACC | 01061916 | |
| MAR 86 MI | ECACC | 01061917 | |
| DMS 53 | ATTC | CRL-2062 | O. S Pettengill; |
| | ecacc | 95062823 | G. Sorensen |
| DMS 79 | ATTC | CRL-2049 | |
| | ecacc | 95062824 | |
| DMS 92 | ecacc | 950662825 | |
| DMS 114 | ATTC | CRL-2066 | |
| DMS 153 | ATTC | CRL-2064 | |
| | ecacc | 95062827 | |
| DMS 273 | ecacc | 95062830 | |
| SHP-77 | Ecacc | 98110201 | A. M. Koros |
| | ATTC | CRL-2195 | |
| NCI-H2171 | ATTC | CRL-5929 | A. F. Gazdar; |
| | | | J. D. Minna |
| NCI-H2195 | | CRL-5931 | |
| NCI N417 | | CRL-5809 | |
| NCI-H2196 | | CRL-5932 | |
| NCI-H2198 | | CRL-5933 | |
| NCI-H2227 | | CRL-5934 | |
| NCI-H2286 | | CRL-5938 | |
| NCI-H2330 | | CRL-5940 | |
| NCI-H735 | | CRL-5978 | |
| NCI-H1339 | | CRL-5979 | |
| NCI-H1963 | | CRL-5982 | |
| NCI-H2107 | | CRL-5983 | |
| NCI-H2108 | | CRL-5984 | |
| NCI-H1304 | | CRL-5862 | |
| NCI-H1341 | | CRL-5864 | |
| NCI-H1417 | | CRL-5869 | |
| NCI-H1436 | | CRL-5871 | |
| NCI-H1522 | | CRL-5874 | |
| NCI-H1618 | | CRL-5879 | |
| NCI-H1672 | | CRL-5886 | |
| NCI-H1694 | | CRL-5888 | |
| NCI-H1836 | | CRL-5989 | |
| NCI-H1870 | | CRL-5901 | |
| NCI-H1876 | | CRL-5902 | |
| NCI-H1882 | | CRL-5903 | |
| NCI-H1926 | | CRL-5905 | |
| NCI-H1930 | | CRL-5906 | |
| NCI-H1994 | | CRL-5910 | |
| NCI-H2029 | | CRL-5913 | |
| NCI-H2059 | | CRL-5916 | |
| NCI-H2066 | | CRL-5917 | |
| NCI-H2081 | | CRL-5920 | |
| NCI-H2141 | | CRL-5927 | |
| NCI-H211 | | CRL-5824 | |
| NCI-H220 | | CRL-5825 | |
| NCI-H250 | | CRL-5828 | |
| NCI-H524 | | CRL-5831 | |
| NCI-H592 | | CRL-5832 | |
| NCI-H711 | | CRL-5836 | |
| NCI-H719 | | CRL-5837 | |
| NCI-H740 | | CRL-5840 | |
| NCI-H748 | | CRL-5841 | |
| NCI-H774 | | CRL-5842 | |
| NCI-H841 | | CRL-5845 | |
| NCI-H847 | | CRL-5846 | |
| NCI-H865 | | CRL-5849 | |
| NCI-H1048 | | CRL-5853 | |
| NCI-H1059 | | CRL-5854 | |
| NCI-H1092 | | CRL-5855 | |
| NCI-H1105 | | CRL-5856 | |
| NCI-H1184 | | CRL-5858 | |
| NCI-H1238 | | CRL-5859 | |
| NCI-H1284 | | CRL-5861 | |
| NCI-H1688 | | CRL-257 | |
| NCI-H187 | | CRL-5804 | |
| NCI-H378 | | CRL-5808 | |
| NCI-H526 | | CRL-5811 | |
| NCI-H660 | | CRL-5813 | |
| NCI-H889 | | CRL-5817 | |
| NCI-H60 | | CRL-5821 | |
| NCI-H196 | | CRL-5823 | |
| NCI-H446 | | HTB-171 | |
| NCI-H209 | | HTB-172 | |
| NCI-H146 | | HTB-173 | |
| NCI-H82 | | HTB-175 | |
| NCI-H460 | | HTB-177 | |
| NCI-H345 | | HTB-180 | |
| NCI-H510A | | HTB-184 | |
| NCI-128 | | HTB-120 | A. F. Gazdar |
| NCI-446 | | HTB-171 | |
| NCI H 69 | | HTB-119 | |
| SW 1271 | | CRL-2177 | W. McCombs |

The methods for example involve at least 4, such as at least 5, for example at least 6, such as at least 8, for example at least 10, such as at least 12, for example at least 14, such as at least 16, for example at least 18, such at least 20, for example 21, such at least 25, for example at least 30, such as at least 40, for example at least 50, such as at least 60, for example at least 70, such as around 79 malignant cell lines selected from the group consisting of cell lines mentioned in table 1.

In one preferred embodiment of the invention the method involve all of the cell lines DMS 53, DMS 70, DMS 92, DMS 114, DMS 153, DMS 273, NCI 417 and NCI H69.

The cell lines may be cultured by any suitable means, for example the cell lines may be cultured in an in vitro cell culture under suitable conditions known to the person skilled in the art. In one embodiment of the present invention, one or more cell lines are cultured in vivo in an animal as a xenograft. The animal may be any suitable animal, preferably a mammal, more preferably a rodent, most preferably a mouse. An example of how cell lines may be cultured in vivo as a xenograft is given in example 1.

It is also comprised within the present invention that the same cell line may cultured in an in vitro cell culture and may be cultured in vivo.

In generel, in vivo culture conditions i.e. culturing as a xenograft in an animal more closely resembles a natural occurring tumour or cancer and hence it is usually preferred that at least one, such as at least 2, for example at least 3, such as at least 4, for example at least 5, such as at least 6, for example at least 7, such as at least 8 cell lines are cultured in vivo. More preferably, in the range of 1 to 79, such as 2 to 70, for example 3 to 60, such as 4 to 50, for example 5 to 40, such as 6 to 30, for example 7 to 20 cell lines are cultured in vivo. Even more preferably around 8 cell lines are cultured in vivo.

Preferably, the cell lines cultured in vivo are selected from the group of cell lines mentioned in table 1, even more preferably, the cell lines cultured in vivo are selected from the group consisting of CPH 54A, CHP 136A, GLC 3, GLC 14, DMS 273, NCI-H69, NCI-N417 and MAR H24.

Normal tissues are tissues, which are non-malignant. Preferably, such tissue is derived from an individual, which do not suffer from a premalignant and/or malignant condition. More preferably, the normal tissues are mammalian tissues, even more preferably, the tissues are human tissues. Yet more preferably, the tissues are selected from the group-consisting of liver, heart, kidney, lung, adrenal gland, colon, pancreas, small intestine, spleen, skeletal muscle, trachea, prostate, placenta, salivary gland, testes, leucocytes, leucocytes, brain, adipose tissue, bladder, breast, cervix, esophagus, larynx, ovary, rectum, skin, spinal cord, stomach, thymus, thyroid and uterus. Even more preferably, the tissues are selected from the group consisting of brain, adrenal gland, lung, kidney, heart, trachea, prostate, salivary gland, thyroid, liver, pancreas, spleen, small intestine, skeletal muscle, colon, stomach and testes. Most preferably the tissues are selected from the group consisting of lung, liver, heart and kidney.

Preferably, the method involves at least 3, for example at least 4, such as at least 5, for example at least 6, such as at least 8, for example at least 10 total RNA samples.

The method may be any method suitable to compare the level of mRNA found in malignant cell lines with the levels of mRNA found in normal tissues known to the person skilled in the art. In general such method involves purification of either mRNA or total RNA. Purification of RNA may be performed according to any standard method known to the person skilled in the art as for example described in Sambrook et al, 1989 or herein below in the examples.

The RNA samples may be compared by a number of different techniques. Any suitable technique may be applied with the present invention. For example the RNA samples can be compared by differential display or by subtractive hybridisation. Furthermore, techniques involving hybridisation of labelled RNA or cDNA pools with known nucleic acid sequences are suitable with this invention. The known nucleic acids may for example be immobilised on a solid support prior to hybridisation for example on a membrane, such as a nitrocellulose membrane, or the solid support may be of plastic or of glass.

The labelled RNA or cDNA may be labelled with any directly or indirectly detectable label for example an enzyme, a radioactive isotope, chromophore, a fluorescent group or a heavy metal. Furthermore, the label may be one part of a pair of binding partners, wherein the second part is detectable, either directly or indirectly. For detection of an indirectly detectable label, it is possible to use a “sandwich” system, such as be one part of a pair of binding partners is recognised by the second part, which is in turn recognised by the first part, which may again be recognised by the second part. In every step the first and/or second part may be labelled. Examples of pairs of binding partners are antigen/antibodies or biotin/streptavidin. However, any other suitable pair can also be employed with the present invention.

In one embodiment of the present invention the method comprises the steps of:
i) Isolating RNA comprising mRNA from the malignant cell lines
ii) Preparing cDNA populations from said RNA, wherein one cDNA population is prepared from RNA isolated from one cell line or one tissue sample
iii) Labelling each cDNA population with a detectable label
iv) Providing solid supports on which an array of known nucleic acid sequences has been immobilised
v) Incubating each cDNA population with a solid support under conditions which allows for hybridisation
vi) Detecting said detectable label on the solid supports

Preferably said detectable label is an indirectly detectable label, more preferably the label is one part of a pair of binding partners, wherein the second part is detectable, either directly or indirectly. Most preferably the label is biotin. The biotin can be detected with a labelled streptavidin species, preferably a fluorescently labelled streptavidin. More preferably, the streptavidin may furthermore associate with an anti-streptavidin antibody labelled with biotin, which in turn maybe detected by labelled streptavidin, preferably fluorescently labelled. The fluorescent label may for example be phycoerythrin or any other suitable fluorescent label.

In one preferred embodiment the solid support is a glass plate. Preferably, at least 1000, such as at least 5000, for example at least 10,000, such as at least 50.000, such as at least 100.000, for example at least 150.000, such as at least 200.000, for example around 240.000 different known nucleic acid sequences are immobilised on the solid support. These nucleic acid sequences may all be derived from different genes, however, more preferably, each gene is represented by more than one, such as more than 2, for example more than 4, such as more than 7, for example more than 10, such as more than 15, for example more than 20, preferably around 20 different nucleic acid sequences.

In one embodiment of the present invention the RNA samples may be compared by a CHIPS analysis or a GeneChips analysis. The terms CHIPS analysis and GeneChips analysis are used interchangeably throughout the description. An example of how to perform a CHIPS analysis is given in example 1.

Suitable cell surface molecules are selected according to several criteria. Preferably, there is a difference between the amount of mRNA expressed in one or more cell lines used in the method according to the present invention and the amount of mRNA expressed in one or more tissues according to the present invention. Preferably the difference is at least 1.1 fold, such as 1.2 fold, such as 1.5-fold, such as 1.75 fold, such as 2-fold, such as 2.5 fold, such as at least 3-fold, for example at least 4-fold, such as least 5-fold, for example at least 7.5 fold, such as least 10 fold difference in mRNA expression. In one preferred embodiment the difference is an in principle unlimited number of fold, such as there is no detectable mRNA expressed in one or more cell lines and mRNA is detectable in one or more normal tissues, or there is no detectable mRNA expressed in one or more normal tissues and mRNA is detectable in one or more cell lines.

Furthermore, there is preferably essentially no difference in the amount of mRNA expressed in at least two, such as at least 3, for example at least 4, such as at least 5, for example at least 6, such as at least 8, for example at least 10, such as at least 12, for example at least 14, such as at least 16, for example at least 18, such at least 20, for example 21, such at least 25, for example at least 30, such as at least 40, for example at least 50, such as at least 60, for example at least 70, such as around 79 malignant cell lines used in the method according to the present invention.

Additionally, there is preferably essentially no difference in the amount of mRNA expressed in at least two, such as at least 3, for example at least 4, such as at least 5, for example at least 6, such as at least 8, for example at least 10 tissue samples, from which total RNA was used according to the methods of the present invention.

Nucleic acid sequences encoding for potential cell surface molecules are selected from nucleic acid sequences that full fill the above criteria. In one particular preferred embodiment the potential cell surface molecules are identified as outlined in example 1 and selected according to the criteria described in that example.

To determine what nucleic acid sequences encode potential cell surface molecules different strategies may be employed. For example potential cell surface molecules may be selected according to information available in commonly accessible databases. Such databases may for example be selected from the group consisting of PubMed (NCBI), Nucleotide (NCBI), Protein (NCBI), Structure (NCBI), OMIM (NCBI) and LocusLink (NCBI). NCBI is the abbreviation for National Center for Biotechnology Information. Furthermore, potential cell surface molecules may be selected based on the presence of one or more of selected terms in name of the potential cell surface molecules. For example said terms may be selected from the group consisting of receptor, membrane, adhesion, integrin, surface, antigen, syndecan, transport, channel, hormone, binding, glycoprotein, matrix, CAM, desmosome, gap junction, delta, immunoglobulin, MHC, CD, HSPG, CSPG, integral and notch.

Alternatively, nucleic acid sequences encoding for potential cell surface molecules are selected according to sequence homology with known cell surface molecules. Nucleic acid sequences encoding potential cell surface molecules should have at least 20%, for example at least 22.5%, such as at least 25%, for example at 27.5%, such as at least 30% sequence identify with nucleic acid sequences encoding known cell surface molecules.

The nucleic acid sequences encoding potential cell surface molecules may also be selected based on sequence homology with domains comprised within known; cell surface molecules. Preferably, there is at least 20%, for example at least 22.5%, such as at least 25%, for example at 27.5%, such as at least 30%, for example at least 32.5%, such as at least 35%, for example at least 37.5%, such as at least 40%, for example at least 42.5%, such as at least 45%, for example at least 47.5%, such as at least 50% sequence identify between domains of the nucleic acids encoding potential cell surface molecules and nucleic acid sequences encoding domains of known cell surface molecules.

Nucleic acid sequences encoding potential cell surface molecules may also be selected based on that the potential cell surface molecules comprise a domain, which is often associated with the cell surface. Such a domain may for example be selected from the group consisting of hydrophobic regions and potential glycosylation sites.

In one embodiment of the present invention candidate cell surface molecules have been identified by a Chips analysis. Suitable cell surface molecules may then be selected based on several criteria. For example cell surface molecules, which scored present (P) in the absolute call and with an Average difference of for example >10, such as >20, for example >40, such as >50 may be included. Furthermore, it is possible to make a point system to identify suitable cell surface molecules. For example a gene encoding a cell surface molecule may be set to score a number of points, such as one point for each cell line or tissue expressing the gene. The total scores for each gene may be summarised for normal tissue and the SCLC cell lines, respectively. Genes may then be selected, which were scored present in at least 3, such as 4, for example 5, such as 6, for example 7, such as 8, for example 9, such as 10, for example more than 10 of the SCLC lines. A preferred method of selecting cell surface molecules is described in example 1.

The present invention also provides methods for identifying first nucleic acid sequences, which are capable of directing expression of second nucleic acid sequences operably linked thereto. These methods involves identifying second nucleic acid sequences, which are expressed at a level, which is different in malignant cells compared with normal cells.

Accordingly, there is preferably a difference between the amount of second nucleic acid sequence mRNA expressed in one or more cell lines and the amount of second nucleic acid sequence mRNA expressed in one or more tissues. More preferably, the difference is at least 1.1 fold, such as 1.2 fold, such as 1.5-fold, such as 1.75 fold, such as 2-fold, such as 2.5 fold, such as at least 3-fold, for example at least 4-fold, such as least 5-fold, for example at least 7.5 fold, such as least 10 fold difference in mRNA expression.

In one preferred embodiment the difference is an in principle unlimited number of fold, such as there is no detectable second nucleic sequence mRNA expressed in one or more cell lines and said mRNA is detectable in one or more normal tissues, or there is no detectable second nucleic acid sequence mRNA expressed in one or more normal tissues and said mRNA is detectable in one or more cell lines.

Furthermore, there is preferably essentially no difference in the amount of second nucleic acid sequence mRNA expressed in at least two, such as at least 3, for example at least 4, such as at least 5, for example at least 6, such as at least 8, for example at least 10, such as at least 12, for example at least 14, such as at least 16, for example at least 18, such at least 20, for example 21, such at least 25, for example at least 30, such as at least 40, for example at least 50, such as at least 60, for example at least 70, such as around 79 malignant cell lines used with the methods of the present invention.

Additionally, there is preferably essentially no difference in the amount of second nucleic acid sequence mRNA expressed in at least two, such as at least 3, for example at least 4, such as at least 5, for example at least 6, such as at least 8, for example at least 10 normal tissue samples.

In one particularly preferred embodiment the second nucleic acid sequences are identified according to the method described in example 1. Most preferably, the criteria outlined in that example are applied to select useful second nucleic acid sequences.

In one embodiment of the present invention candidate promoters have been identified by a Chips analysis. Suitable promoters may then be selected based on several criteria based on expression level of the gene which the promoter controls.

For example only genes, which scored present (P) in the absolute call and with an Average difference of >10, such as >20, for example >30, such as >40, for example >50 (level of expression) may be included included. A point scoring system as described herein above may be used. Genes that scored present in for example at least 3, such as at least 4, for example at least 5, such as at least 6, for example at least 7, such as at least 8, for example at least 9, such as at least 10, for example at least 11, such as at least 12 SCLC lines may for example be selected. If a gene scores present in one or more normal tissues, the median Average difference value of the SCLC cell lines should preferably be 4 times or more above the median Average difference value of the normal tissue. Preferably promoters with an Average differences of expression for normal tissues <50 and for SCLC>100 are selected. More preferably, promoters with an Average differences of expression for normal tissues <50 and for SCLC>200. Most preferably, promoters with an Average differences of expression for normal tissues <50 and for SCLC>400 Alternatively, promoters with an Average differences of expression in SCLC >8 times higher than for normal tissue may be selected. A preferred method of selecting cell surface molecules is described in example 1.

Once second nucleic acid sequences have been identified according to the above mentioned criteria, it is possible to identify first nucleic acid sequences operably linked to the second nucleotide sequences. This can be done according to any standard method known to the person skilled in the art. For example it is possible to take advantage of known human genome sequences.

Cell Surface Molecules

A cell surface molecule according to the present invention is any molecule naturally associated with the cell surface. Cell surface molecules may not be associated with the cell surface throughout their life time, but may be associated with the cell surface only at specific times. Cell surface molecules within the scope of the present invention may be any kind of molecule associated with the cell surface, however the cell surface molecules according to the present invention preferably comprise a polypeptide.

However, in other preferred embodiments of the invention, cell surface molecules include for example molecules that are associated directly with the cell surface for example via a transmembrane domain, a membrane anchoring domain or a covalently linked group, which can associate with the membrane such as for example a lipophilic group. A lipophilic group may for example be a glycosyl-phosphatidylinositol group (GPI). However, it also includes molecules which are indirectly associated with the cell surface for example molecules that can associate with other cell surface molecules which are either directly or indirectly associated with the cell surface.

In general a cell surface molecule comprise at least one extracellular domain, however a cell surface molecule may comprise more than one extracellular domain such as 2, for example 3, such as 4, for example 5, such as 6, for example 7, such as 8, for example 9, such as 10, for example more than 10 extracellular domains.

Frequently, cell surface molecules are glycosylated polypeptides.

In one preferred embodiment of the present invention, cell surface molecules are capable of associating with specific binding partners, and capable of internalising said specific binding partners upon association, i.e. after association between binding partner and cell surface molecule, the binding partner is transferred to the interior of the cell expressing the cell surface molecule. Frequently, the binding partner will be internalised by receptor mediated endocytosis, but other mechanisms are also possible and within the scope of the present invention. Cell surface molecules capable of internalising a binding partner may for example be useful for radio-, toxin- or gene therapy or cancer vaccines.

In another preferred embodiment of the present invention, cell surface molecules are capable of associating with specific binding partners at the cell surface, but are not capable of internalising said specific binding partners. Non-internalising cell; surface molecules may for example be useful for radio-therapy and cancer vaccines.

Accession numbers from GenBank and names of preferred cell surface molecules are given in table 2.

TABLE 2

| | Cell surface | | SEQ ID | SEQ ID |
|---|---|---|---|---|
| Accession | molecule | Gene name | (cDNA/DNA) | (protein) |
| M11507 | Transferrin receptor | Human transferrin receptor mRNA, complete | 1 | 2 |
| | | cds. | | |
| X01060 | Transferrin receptor | Human mRNA for transferrin receptor. | 3 | 4 |
| AB015633 | HP10481 | Homo sapiens mRNA for type II membrane | 5 | 6 |
| | | protein, complete cds clone: HP10481. | | |
| M14219 | PG40 | Human chondroitin/dermatan sulfate | 7 | 8 |
| | | proteoglycan (PG40) core protein mRNA, | | |
| | | complete cds. | | |
| AF064801 | TRC8 | Homo sapiens multiple membrane spanning | 9 | 10 |
| | | receptor TRC8 (TRC8) mRNA, complete cd | | |
| M29960 | TR2-11 | Human steroid receptor (TR2-11) mRNA, | 11 | 12 |
| | | complete cds. | | |
| X69398 | OA3 antigenic surface | H. sapiens mRNA for OA3 antigenic surface | 13 | 14 |
| | determinant | determinant | | |
| X53586 | Integrin alpha 6 | Human mRNA for integrin alpha 6. | 15 | 16 |
| M34480 | GPIIb | Human platelet glycoprotein IIb (GPIIb) | 17 | 18 |
| | | mRNA, complete cds | | |
| M14648 | Vitronectin receptor | Human cell adhesion protein (vitronectin) | 19 | 20 |
| | alpha subunit; also | receptor alpha subunit; ITGAV mRNA, | | |
| | designated ITGAV | complete cds. | | |
| AF032108 | Integrin alpha-7 | Homo sapiens integrin alpha-7 mRNA, | 21 | 22 |
| | | complete cds. | | |
| M35011 | Integrin beta-5 subunit | Human integrin beta-5 subunit mRNA, | 23 | 24 |
| | | complete cds. | | |
| X53002 | | Human mRNA for integrin beta-5 subunit. | 25 | 26 |
| L25851 | Integrin alpha E pre- | Homo sapiens integrin alpha E precursor; | 27 | 28 |
| | cursor; also designated | ITGAE, mRNA, complete cds. | | |
| | ITGAE | | | |
| S66213 | Integrin alpha 6B | Integrin alpha 6B [human, mRNA Partial, | 29 | 30 |
| | | 528 nt]. | | |
| X06256 | Integrin alpha 5 subunit | Human mRNA for integrin alpha 5 sub-unit. | 31 | 32 |
| M59911 | Integrin alpha-3 chain | Human integrin alpha-3 chain mRNA, | 33 | 34 |
| | | complete cds. | | |
| S59184 | RYK | RYK = related to receptor tyrosine kinase | 35 | 36 |
| | | [human, hepatoma, mRNA, 3068 nt]. | | |
| U50939 | Amyloid precursor protein- | Human amyloid precursor protein-binding | 37 | 38 |
| | binding protein 1 | protein 1 mRNA, complete cds. | | |
| U95822 | Putative transmembrane | Human putative transmembrane GTPase mRNA, | 39 | 40 |
| | GTPase | partial cds. | | |
| X59408 | Membrane cofactor protein | H. sapiens, gene for Membrane cofactor | 41 | |
| | | protein. | | |
| L20859 | GLVR1 | Human leukemia virus receptor 1 (GLVR1) | 42 | 43 |
| | | mRNA, complete cds. | | |
| D64154 | Mr 110,000 antigen | Human mRNA for Mr 110,000 antigen, | 44 | 45 |
| | | complete cds. | | |
| | | Inter-Alpha-Trypsin Inhibitor Heavy Chain | | 47 |
| | | LIKE gene | | |
| Z48199 | Syndecan-1 | H. sapiens syndecan-1 gene (exons 2-5). | 48 | 49 |
| Y18007 | Putative seven trans- | Homo sapiens mRNA for putative seven | 50 | 51 |
| | membrane domain protein | transmembrane domain protein | | |
| Y00815 | LCA-homolog/LAR protein | Human mRNA for LCA-homolog. LAR protein | 52 | 53 |
| | | (leukocyte antigen related) | | |
| X64364 | M6 antigen | H. sapiens mRNA for M6 antigen. | 54 | 55 |
| X62654 | Me491/CD63 antigen | H. sapiens gene for Me491/CD63 antigen. | 56 | 57 |
| U94831 | Multispanning membrane | Homo sapiens multispanning membrane | 58 | 59 |
| | protein | protein mRNA, complete cds. | | |
| U48705 | DDR | Human receptor tyrosine kinase DDR gene, | 60 | 61 |
| | | complete cds. | | |
| M63175 | Autocrine motility | Human autocrine motility factor receptor mRNA. | 62 | 63 |
| | factor receptor | | | |
| AB015631 | Type II membrane protein | Homo sapiens mRNA for type II membrane | 64 | 65 |
| | clone | protein, complete cds, clone: HP10390. | | |
| Y00285 | Insuline-like growth | Human mRNA for insuline-like growth | 66 | 67 |
| | factor II receptor | factor II receptor. | | |
| U01160 | SAS | Human transmembrane 4 superfamily protein | 68 | 69 |
| | | (SAS) mRNA, complete cds | | |
| M33680 | TAPA-1 | Human 26-kDa cell surface protein TAPA-1 | 70 | 71 |
| | | mRNA, complete cds | | |
| M16941 | MHC class II HLA-R7- | Human MHC class II HLA-DR7-associated | 72 | 73 |
| | associated glycoprotein | glycoprotein beta-chain mRNA complete cds. | | |
| | beta-chain | | | |
| J04599 | Bone small proteoglycan I | Human hPGI mRNA encoding bone small | 74 | 75 |
| | biglycan | proteoglycan I biglycan), complete cds | | |
| Y07593 | CAR | H. sapiens mRNA for 46 kDa coxsackievirus | 76 | 77 |
| | | and adenovirus receptor (CAR) protein. | | |
| U73682 | MEA11 | Human meningioma-expressed antigen | 78 | 79 |
| | | 11 (MEA11) mRNA, partiel cds. | | |
| U19247 | Interferon-gamma | Homo sapiens interferon-gamma receptor | 80 | 81 |
| | receptor alpha chain | alpha chain gene, exon 7 and complete cds. | | |
| X73079 | Polymeric immunoglobulin | Homo sapiens encoding Polymeric immuno- | 82 | 83 |
| | receptor | globulin receptor | | |
| X80818 | Metabotropic glutamate | H. sapiens mRNA for metabotropic glutamate | 84 | 85 |
| | receptor type 4 | receptor type 4. | | |
| AF037339 | CLPTM1 | Homo sapiens cleft lip and palate trans- | 86 | 87 |
| | | membrane protein 1 (CLPTM1) mRNA, | | |
| | | complete cds. | | |
| U10689 | MAGE5a | Human MAGE-5a antigen (MAGE5a) gene, | 88 | 89 |
| | | complete cds. | | |
| U03735 | MAGE-3 | Human MAGE-3 antigen (MAGE-3) gene, | 90 | 91 |
| | | complete cds. | | |
| M77481 | MAGE-1 | Human antigen (MAGE-1) gene, complete cds. | 92 | 93 |
| U10691 | MAGE6 | Human MAGE-6 antigen (MAGE6) gene, | 94 | 95 |
| | | complete cds. | | |
| L33930 | CD24 | Homo sapiens CD24 signal transducer | 96 | 97 |
| | | mRNA, complete cds and 3′region | | |
| M84349 | CD59 | Human transmembrane protein (CD59) gene, | 98 | 99 |
| | | exon 4. | | |
| L00352 | Low density lipoprotein | Human low density lipoprotein receptor | 100 | 101 |
| | receptor | gene, exon 18. | | |
| AF023676 | Lamin B receptor homolog | Homo sapiens lamin B receptor homolog | 102 | 103 |
| | TM7SF2 | TM7SF2 (TM7SF2) mRNA complete cds. | | |
| U41804 | T1/ST2 receptor binding | Human putative T1/ST2 receptor binding | 104 | 105 |
| | protein precursor | protein precursor mRNA, complete cds. | | |
| Y10148 | NTR2 receptor | H. sapiens mRNA for NTR2 receptor. | 106 | 107 |
| U46194 | RAGE-4 | Human renal cell carcinoma antigen RAGE-4 | 108 | 109 |
| | | mRNA, complete putative cds. | | |
| M90683 | HLA-G1 | Human lymphocyte antigen (HLA-G1) mRNA, | 110 | 111 |
| | | complete cds. | | |
| AF104942 | MOAT-C | Homo sapiens ABC transporter MOAT-C | 112 | 113 |
| | | (MOAT-C) mRNA, complete cds. | | |
| AF042792 | Alpha 2 delta calcium | Homo sapiens alpha 2 delta calcium | 114 | 115 |
| | channel subunit isoform I | channel subunit isoform I mRNA, compl. cds. | | |
| Y00636 | LFA-3 | Human mRNA for lymphocyte function | 116 | 117 |
| | | associated antigen-3 (LFA-3). | | |
| X59847 | L1-CAM | H. sapiens mRNA for neural cell adhesion | 118 | 119 |
| | | molecule L1 (HSNCAML1) | | |
| XM010168 | AVPR2 | Arginine-vasopressin receptor (AVPR2) | | 120 |
| | C1 p115 C1 | C1 p115 | | 121 |
| | TE2 | ARD1 N-acetyl transferase related protein | | 122 |
| | RbP | Renin binding protein | | 123 |
| | HCF-1 | Host cell factor 1 | | 124 |
| | IRAK | Interleukin-1-receptor associated kinase | | 125 |
| D29963 | CD151 | Homo sapiens mRNA for CD151, complete cds. | 126 | 127 |
| M60922 | Surface antigen | Human surface antigen mRNA, complete | 128 | 129 |
| | | cds. | | |
| M29273 | MAG | Human myelin-associated glycoprotein | 130 | 131 |
| | | (MAG) mRNA, complete cds. | | |
| U64871 | GPR19 | Human putative G protein-coupled receptor | 132 | 133 |
| | | (GPR19) gene, complete cds. | | |
| L78132 | Pcta-1 | Human prostate carcinoma tumor antigen | 134 | 135 |
| | | (pcta-1) mRNA, complete cds. | | |
| U65011 | PRAME | Human preferentially expressed antigen | 136 | 137 |
| | | of melanoma (PRAME) mRNA, compl. cds. | | |
| X81882 | Vasopressin activated | H. sapiens mRNA for vasopressin activated | 138 | 139 |
| | calcium mobilizing | calcium mobilizing receptor-like protein. | | |
| | receptor-like protein | | | |
| U65416 | MICB | Human MHC class I molecule (MICB) gene, | 140 | 141 |
| | | complete cds. | | |
| Y12505 | Serotonin receptor 5-HT4B | H. sapiens mRNA for serotonin receptor | 142 | 143 |
| | | 5-HT4B, splice variant | | |
| M38690 | CD9 | Human CD9 antigen mRNA, complete cds. | 144 | 145 |
| AF077820 | LDL receptor member LR3 | Homo sapiens LDL receptor member LR3 mRNA, | 146 | 147 |
| | | complete cds. | | |
| U10688 | MAGE-4b | Human MAGE-4b antigen (MAGE4b)gene, | 148 | 149 |
| | | complete cds. | | |
| AF068868 | DR6 | Homo sapiens TNFR-related death receptor-6 | 150 | 151 |
| | | (DR6) mRNA, complete cds. | | |
| D16532 | Very low density lipo- | Human gene for very low density lipoprotein | 152 | 153 |
| | protein receptor | receptor, exon 19 | | |
| M81590 | Serotonin 1D receptor | Homo sapiens serotonin 1D receptor | 154 | 155 |
| | (5-HT1D˜) | (5-HT1D˜) mRNA, complete cds | | |
| M58286 | Tumour necrosis factor | Homo sapiens tumor necrosis factor receptor | 156 | 157 |
| | receptor | mRNA, complete cds. | | |
| AF062006 | HG38 | Homo sapiens orphan G protein-coupled | 158 | 159 |
| | | receptor HG38 mRNA, complete cds | | |
| U09937 | Urokinase-type plasminogen | Human urokinase-type plasminogen receptor, | 160 | 161 |
| | receptor | exon 7. | | |
| M22092 | N-CAM; also designated | Human neural cell adhesion molecule (N-CAM) | 162 | 163 |
| | NCAM1 | gene, exon SEC and partial cds. | | |
| M34641 | FGF receptor | Human fibroblast growth factor (FGF) | 164 | 165 |
| | | receptor-1 mRNA, complete cds. | | |
| M14764 | Nerve growth factor | Human nerve growth factor receptor mRNA, | 166 | 167 |
| | receptor | complete cds. | | |
| U10694 | MAGE-9 | Human MAGE-9 antigen (MAGE9) gene, | 168 | 169 |
| | | complete cds. | | |
| AB026891 | Cystine/glutamate | Homo sapiens mRNA for cystine/glutamate | 170 | 171 |
| | transporter | transporter, complete cds. | | |
| U73304 | CB1 cannabinoid receptor | Human CB1 cannabinoid receptor | 172 | 173 |
| | (CNR1) | (CNR1) gene, complete cds. | | |
| M69245 | PSG | Human pregnancy-specific beta-1 glycoprotein | 174 | 175 |
| | | (PSG) mRNA, comple cds | | |
| AB000712 | CPE receptor | Homo sapiens hCPE-R mRNA for CPE- | 176 | 177 |
| | | receptor, complete cds. | | |
| AF011406 | CRH2R | Homo sapiens corticotropin releasing | 178 | 179 |
| | | hormone receptor type 2 beta isoform | | |
| | | (CRH2R) mRNA, complete cds. | | |
| U50410 | OCI5 | Human heparan sulphate proteoglycan | 180 | 181 |
| | | (OCI5) mRNA, complete cds | | |
| AF016266 | TRAIL receptor 2 | Homo sapiens TRAIL receptor 2 mRNA, | 182 | 183 |
| | | complete cds. | | |
| U87947 | HNMP-1 | Human hematopoietic neural membrane | 184 | 185 |
| | | protein (HNMP-1) mRNA, complete cds. | | |
| J03853 | Kidney alpha-2-adrenergic | Human kidney alpha-2-adrenergic receptor | 186 | 187 |
| | receptor | mRNA, complete cds. | | |
| U10686 | MAGE11 | Human MAGE-11 antigen (MAGE11) gene, | 188 | 189 |
| | | complete cds. | | |
| U25988 | PSG13′ | Human pregnancy-specific glycoprotein | 190 | 191 |
| | | 13 (PSG13′) mRNA, complete cds | | |
| M60459 | Erythropoietin receptor | Human erythropoietin receptor mRNA, | 192 | 193 |
| | | complete cds. | | |
| X15998 | Chondroitin sulphate | H. sapiens mRNA for the chondroitin sulphate | 194 | 195 |
| | proteoglycan versican V1 | proteoglycan versican V1 splice-variant; | | |
| | | precursor peptide. | | |
| U31216 | mGlu1beta | Human metabotropic glutamate receptor | 196 | 197 |
| | | 1 beta (mGluR1beta) mRNA, complete cds. | | |
| X94630 | CD97 | H. sapiens CD97 gene exon 1 (and joined CDS). | 198 | 199 |
| M90657 | L6 | Human tumor antigen (L6) mRNA, complete cds. | 200 | 201 |
| U87459 | NY-ESO-1 | Human autoimmunogenic cancer/testis | 202 | 203 |
| | | antigen NY-ESO-1 mRNA, complete cds. | | |
| S71824 | N-CAM; also designated | N-CAM = 145 kda neural cell adhesion | 204 | 205 |
| | NCAM1 | molecule [human, small cell lung cancer | | |
| | | cell line OS2-R, mRNA, 2960 nt]. | | |
| AE000659 | T-cell receptor alpha delta | Homo sapiens T-cell receptor alpha delta | 206 | 207 |
| | | locus from bases 250472 to 501670 | | 208 |
| | | (section 2 of 5) of the Complete | | 209 |
| | | Nucleotide Sequence. | | 210 |
| | | | | 211 |
| | | | | 212 |
| | | | | 213 |
| | | | | 214 |
| | | | | 215 |
| | | | | 216 |
| | | | | 217 |
| | | | | 218 |
| | | | | 219 |
| | | | | 220 |
| | | | | 221 |
| | | | | 222 |
| M97639 | Ror2 | Human transmembrane receptor (ror2) | 223 | 224 |
| | | mRNA, complete cds. | | |
| M81830 | SSTR2 | Human somatostatin receptor isoform 2 | 225 | 226 |
| | | (SSTR2) gene, complete cds | | |
| AF030339 | VESPR | Homo sapiens receptor for viral semaphorin | 227 | 228 |
| | | protein (VESPR) mRNA, complete cds. | | |
| X02160 | Insulin receptor precursor | Human mRNA for insulin receptor precursor. | 229 | 230 |
| U12255 | IgG Fc receptor | Human IgG Fc receptor hFcRn mRNA, | 231 | 232 |
| | | complete cds. | | |
| X82068 | Glutamate receptor subunit | H. sapiens mRNA for glutamate receptor | 233 | 234 |
| | GluRC | subunit GluRC. | | |
| X75208 | HEK2 | H. sapiens HEK2 mRNA for protein tyrosine | 235 | 236 |
| | | kinase receptor. | | |
| X64116 | PVR | H. sapiens PVR gene for poliovirus | 237 | |
| | | receptor (exon 1), | | |
| | | poliovirus receptor γ | | 238 |
| | | poliovirus receptor β | | 239 |
| | | poliovirus receptor α | | 240 |
| M29540 | CEA | Human carcinoembryonic antigen mRNA | 241 | 242 |
| | | (CEA), complete cds. | | |
| U94888 | CC-chemokine-binding | Homo sapiens CC-chemokine-binding | 243 | 244 |
| | receptor JAB61 | receptor JAB61 mRNA, complete cds. | | |
| M97675 | Ror1 | Human transmembrane receptor (ror1) | 245 | 246 |
| | | mRNA, complete cds. | | |
| M12036 | HER2 | Human tyrosine kinase-type receptor | 247 | 248 |
| | | (HER2) gene, partial cds. | | |
| U87460 | Putative endothelin | Human putative endothelin receptor type | 249 | 250 |
| | receptor type B-like | B-like protein mRNA, complete cds. | | |
| | protein | | | |
| L20852 | GLVR2 | Human leukemia virus receptor 2 (GLVR2) | 251 | 252 |
| | | mRNA, complete cds. | | |
| L05424 | | Human cell surface glycoprotein CD44 | 253 | 254 |
| | | (CD44) gene, 3′ end of long tailed | | |
| | | isoform | | |
| M59040 | CD44 | Human cell adhesion molecule (CD44) | 255 | 256 |
| | | mRNA, complete cds | | |
| U83993 | P2X4 purinoreceptor | Human P2X4 purinoreceptor mRNA, | 257 | 258 |
| | | complete cds. | | |
| XM_015664 | FLJ22357 similar to | Homo sapiens hypothetical protein | 261 | 262 |
| | epidermal growth factor- | FLJ22357 similar to Epidermal growth | | |
| | related protein | factor receptor-related protein | | |
| | | (FLJ22357) mRNA. | | |
| NM_022450 | | Homo sapiens hypothetical protein | 259 | 260 |
| | | FLJ22357 similar to Epidermal growth | | |
| | | factor receptor-related protein | | |
| | | (FLJ22357) mRNA. | | |
| M84562 | FPRL1 | Human formyl peptide receptor-like re- | 263 | 264 |
| | | ceptor (FPRL1) mRNA, complete cds | | |
| M34309 | HER3 | Human epidermal growth factor receptor | 265 | 266 |
| | | (HER3) mRNA, complete cds. | | |
| M83664 | HLA-DP | Human MHC class II lymphocyte antigen | 267 | 268 |
| | | (HLA-DP) beta chain mRNA, compl. cds. | | |
| AF025998 | Atrial natriuretic peptide | Homo sapiens atrial natriuretic peptide | 269 | 270 |
| | clearance receptor | clearance receptor (ANPRC) mRNA, | | |
| | | complete cds. | | |
| XM_006034 | Gastrin/CCK-B receptor | Homo sapiens cholecystokinin B receptor | 271 | 272 |
| | | (CCKBR), mRNA. | | |
| M73482 | Neuromedin B receptor | Human neuromedin B receptor (NMB-R) | 273 | 274 |
| | | mRNA, complete cds. | | |
| NM_001496 | GFRA3 | Homo sapiens GDNF family receptor | 275 | 276 |
| | | alpha 3 (GFRA3), mRNA. | | |
| XM_010317 | GRPR | Homo sapiens gastrin-releasing peptide | 277 | 278 |
| | | receptor (GRPR), mRNA. | | |
| U92459 | Metabotropic glutamate | Human metabotropic glutamate receptor | 279 | 280 |
| | receptor 8; also | 8 (GluR8) mRNA, complete cds.; GRM8 | | |
| | designated GRM8 or GluR8 | | | |
| XM_007840 | CDH1 | Homo sapiens cadherin 1, type 1, E- | 281 | 282 |
| | | cadherin (epithelial)(CDH1), mRNA. | | |
| XM_016157 | CDH2 | Homo sapiens cadherin 2, type 1, N- | 283 | 284 |
| | | cadherin (neuronal)(CDH2), mRNA. | | |
| XM_005591 | TGFBR1 | Homo sapiens transforming growth fac- | 285 | 286 |
| | | tor, beta receptor I (activin A receptor | | |
| | | type II-like kinase, 53 kD) (TGFBR1), mRNA. | | |
| XM_003094 | TGFBR2 | Homo sapiens transforming growth factor, | 287 | 288 |
| | | beta receptor II (70-80 kD) (TGFBR2), mRNA. | | |
| XM_001924 | TGFBR3 | Homo sapiens transforming growth factor, | 289 | 290 |
| | | beta receptor III (betaglycan, 300 kD) | | |
| | | (TGFBR3), mRNA. | | |
| NM_000875 | IGF1R | Homo sapiens insulin-like growth factor 1 | 291 | 292 |
| | | receptor (IGF1R), mRNA. | | |
| X00588 | Precursor of epidermal | Human mRNA for precursor of epidermal | 293 | 294 |
| | growth factor receptor | growth factor receptor. | | |
| Z75190 | LRP8 | Homo sapiens apolipoprotein E receptor | 295 | 296 |
| | | 2 (APOER2), also designated LRP8; mRNA | | |
| U62434 | CHRNA 5 | Nicotinic acetylcholine receptor alpha5 | 297 | 298 |
| | | subunit (CHRNA 5); mRNA | | |
| U19878 | TMEFF1 | Transmembrane protein with EGF-like | 299 | 300 |
| | | and two follastyatin-like domains 1 | | |
| | | (TMEFF1); mRNA | | |
| L20814 | GRIA2; also designated | Human glutamate receptor 2 (HBGR2); | 301 | 302 |
| | GluR2 | also designated GluR2 or GRIA2; complete | | |
| | | coding sequence | | |
| AL008583 | NPTXR | Neuronal pentraxin receptor (NPTXR); | 303 | 304 |
| | | DNA sequence | | |

Even more preferred cell surface molecules according to the present invention are receptors which belong to one of the following groups:
Members of receptor tyrosine kinases
Members of the integrin family
Members of the immunoglobulin superfamily adhesion molecules
Members of the heparan sulfate proteoglycan family
Members of the chondroitin sulfate proteoglycan family
Members of the MAGE family
Members of the RAGE family
Members of the low density lipoprotein receptor family
Members of the cadherin adhesion molecules
Members of the metabotropic glutamate receptors
Members of the steroid hormone families
Members of the seven transmembrane receptor family
Atrial natriuretic peptide clearance receptor
GFRA3
Transferrin receptor
Members of the serine/threonine kinase receptors

Yet more preferred cell surface molecules according to the present invention are cell surface molecules selected from the group consisting of NCAM1, NPTXR, LRP8, CHRNA5, GRIA2, GRMB, ITGAV, ITGAE, TNFRSF12, L1CAM, GPR49 and TMEFF1.

In one preferred embodiment of the present invention, cell surface molecules are capable of internalising specific binding partners upon association (see herein above). Preferred cell surface molecules according to the present invention capable of internalising a binding partner may be selected from the group consisting of NCAM1, NPTXR, LRP8 and CHRNA5.

In another preferred embodiment of the present invention, cell surface molecules are not capable of internalising their specific binding partners. Preferred cell surface molecules according to the present invention not capable of internalising binding partner(s) may be selected from the group consisting of GRIA2, GRM8, ITGAV and ITGAE

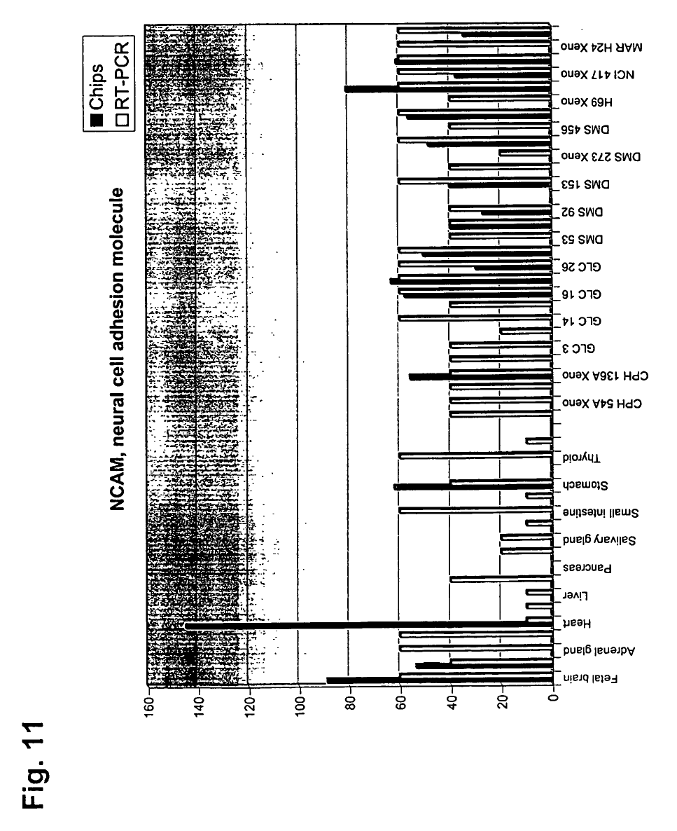

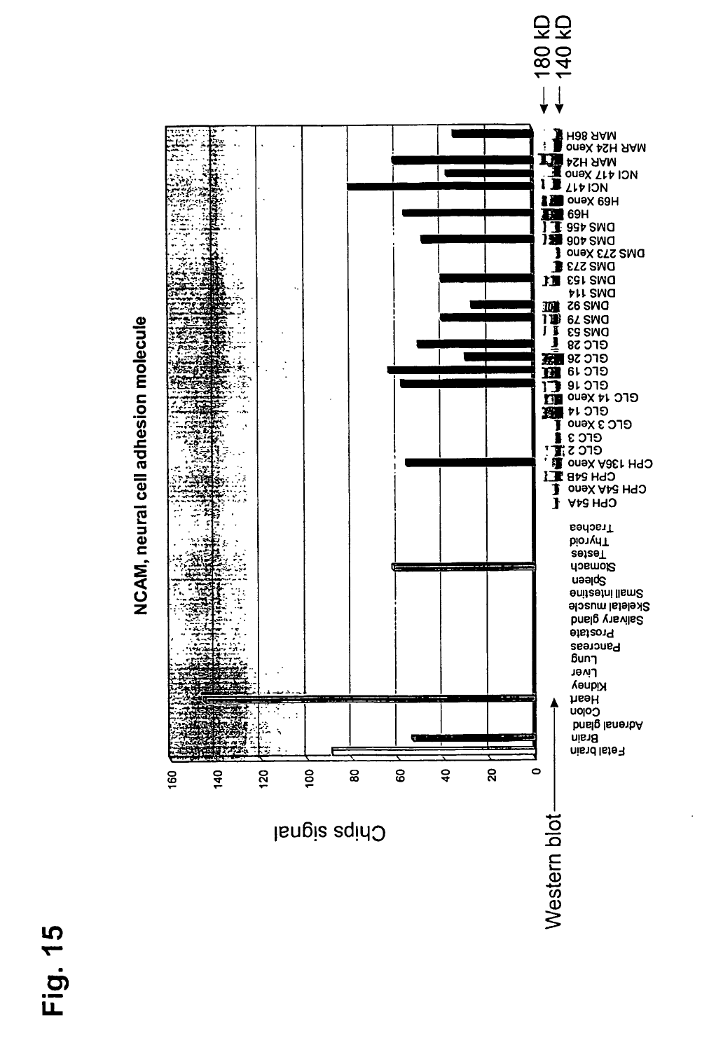

One especially preferred cell surface molecule according to the present invention is NCAM1 (NCAM, neural cell adhesion molecule, N-CAM, CD56)._NCAM1 is highly, expressed in most SCLC. The expression of NCAM1 has been shown by for example CHIPS analysis, RT-PCR and western blotting (see example 1, FIG. 11 and FIG. 15). NCAM1 is capable if internalising binding partners. Binding partners capable of associating with NCAM1 are described herein below. NCAM1 has been shown to be internalised in astrocytes (Minana et al., 2001) and has been demonstrated capable of very efficient internalisation of a very large molecule complex consisting of anti-NCAM1 antibody-Protein A-Streptavidine-Biotin-β-galactosidase enzyme (Yu et al., 2000).

Another preferred cell surface molecule according to the present invention is NPTXR (Neuronal pentraxin receptor, NPR). NPTXR is expressed in most SCLC.

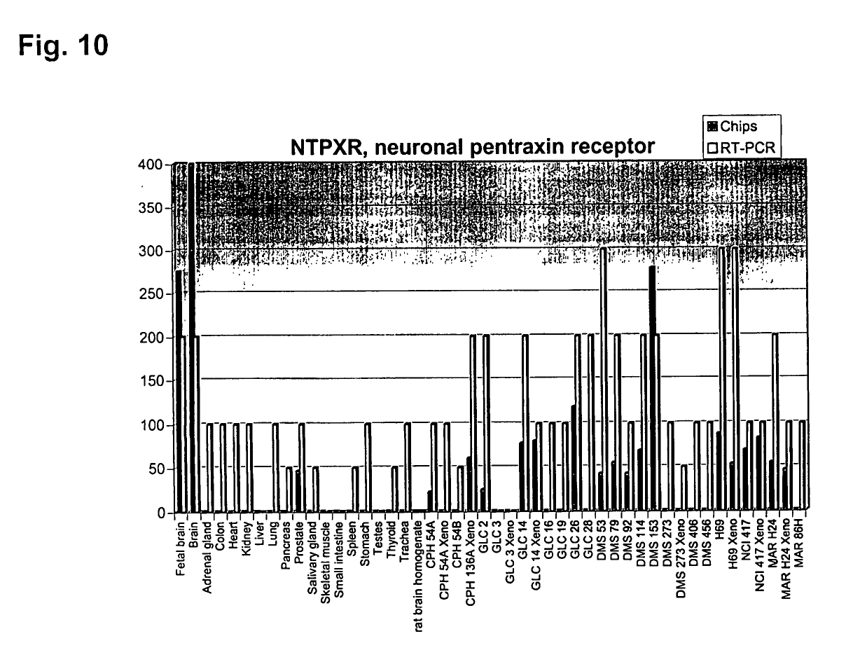

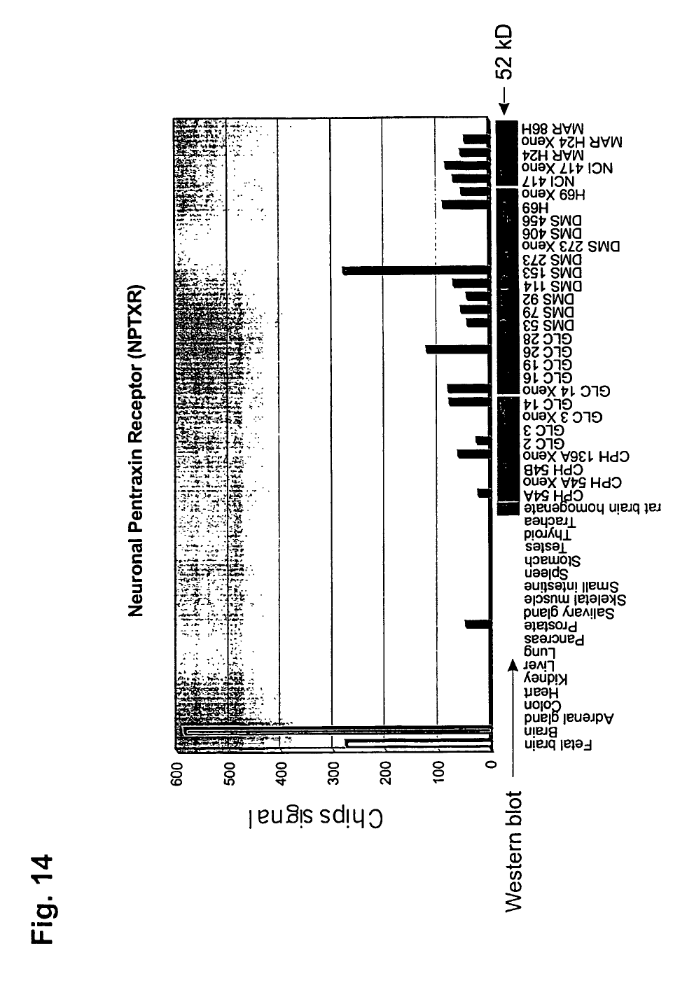

The expression of NPTXR has been demonstrated by for example CHIPS analysis, RT-PCR and western blotting (see example 1, FIG. 10 and FIG. 14). NPTXR is a highly internalising receptor. Binding partners capable of associating with NPTXR are described herein below.

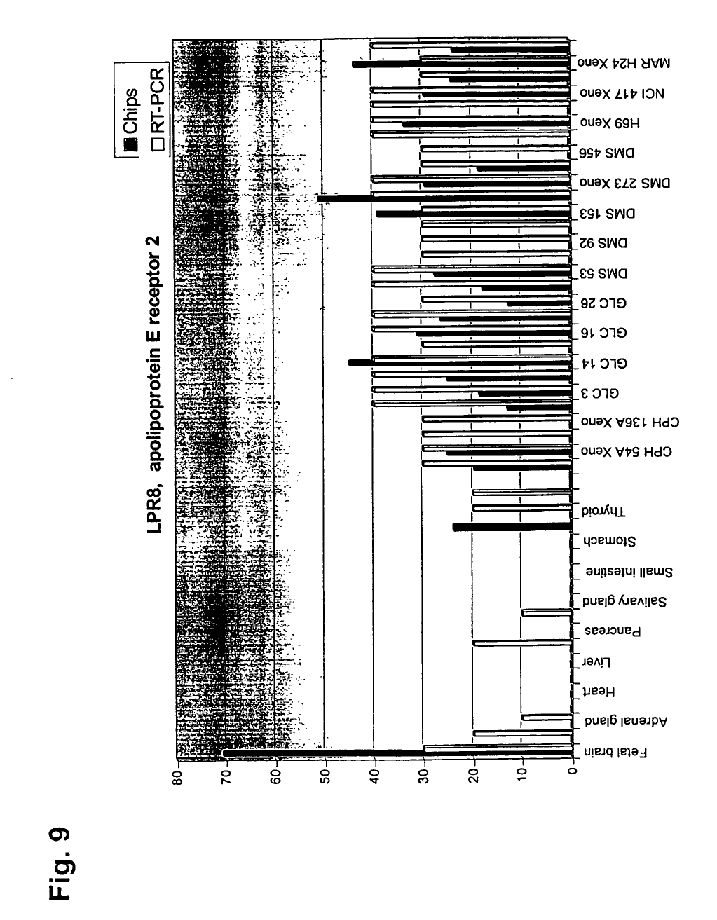

Yet another preferred cell surface molecule according to the present invention is LRP8 (low density lipoprotein receptor related protein, apolipoprotein E receptor 2). LRP8 is expressed in alt tested SCLC. The expression of LRP8 has been demonstrated by for example CHIPS analysis and RT-PCR (see example 1 and FIG. 9). LRP8 is a highly internalising receptor. Binding partners capable of associating with LRP8 are described herein below.

Yet another preferred cell surface molecule according to the present invention is CHRNA5 (nicotinic acetylcholine receptor alpha5 subunit). Binding partners capable of associating with CHRNA5 are described herein below.

Yet another cell surface molecule that may be used with the present invention is L1CAM (neural cell adhesion molecule L1). L1 is known to be able to internalise binding partners. Binding partners capable of associating with L1CAM are described herein below.

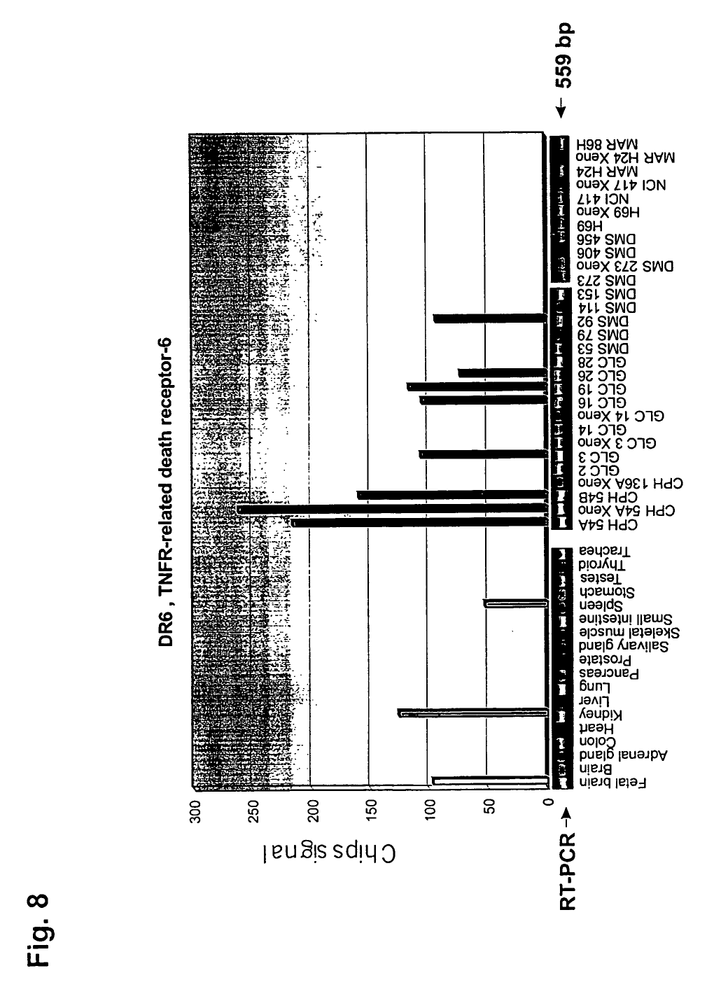

Yet another preferred cell surface molecule according to the present invention is TNFRSF12 (DR6, tumor necrosis factor receptor superfamily member 21). TNFRSF12 is expressed in most SCLC. The expression of TNFRSF12 has been demonstrated by for example CHIPS analysis and RT-PCR (see example 1 and FIG. 8). Other members of the family to which TNFRSF12 belong are capable of internalising binding partners.

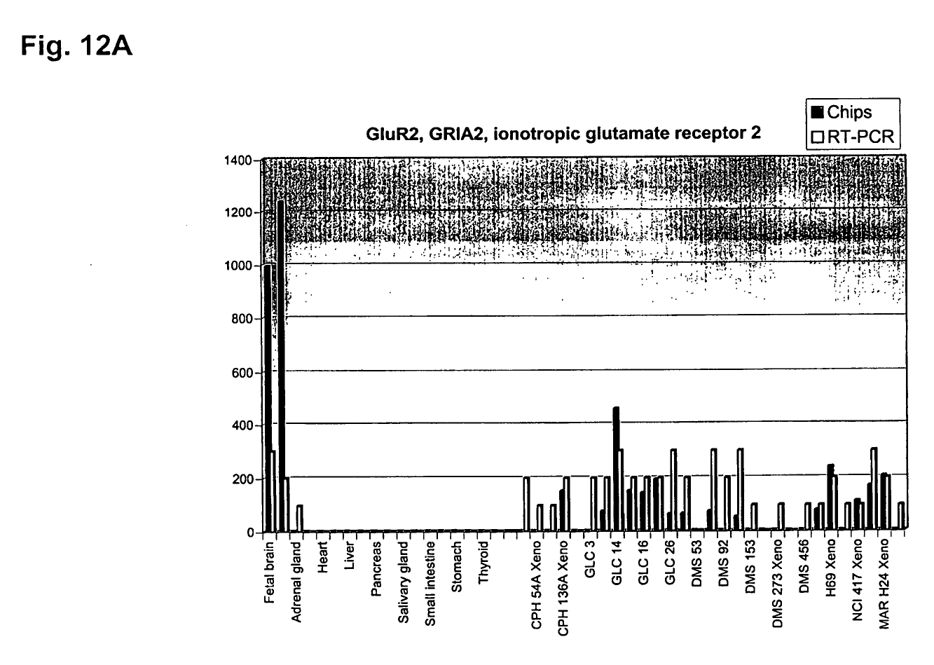

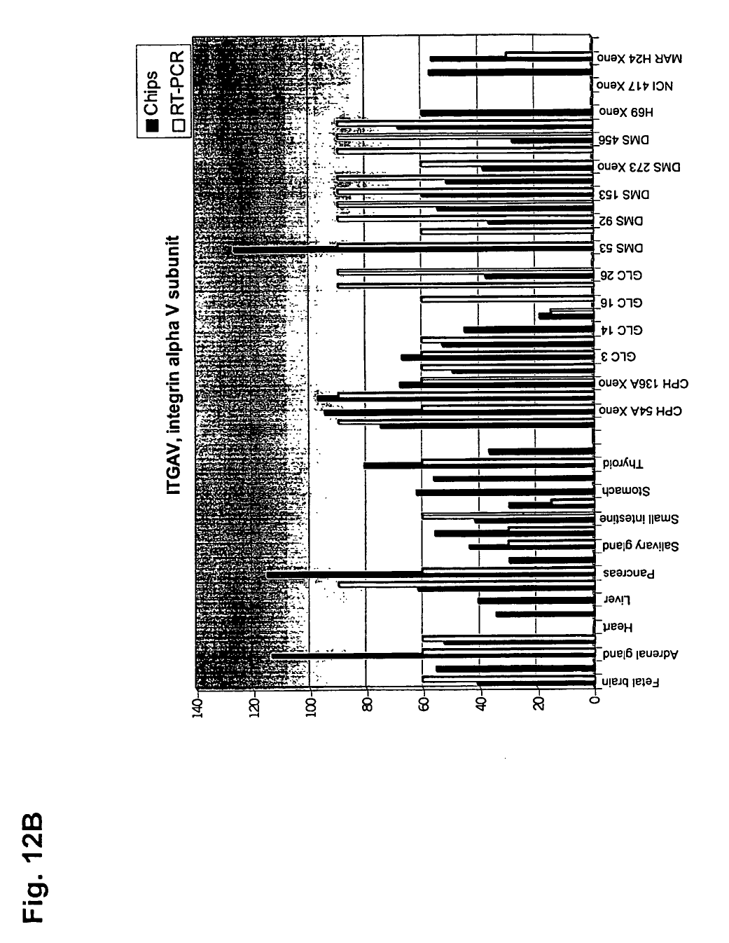

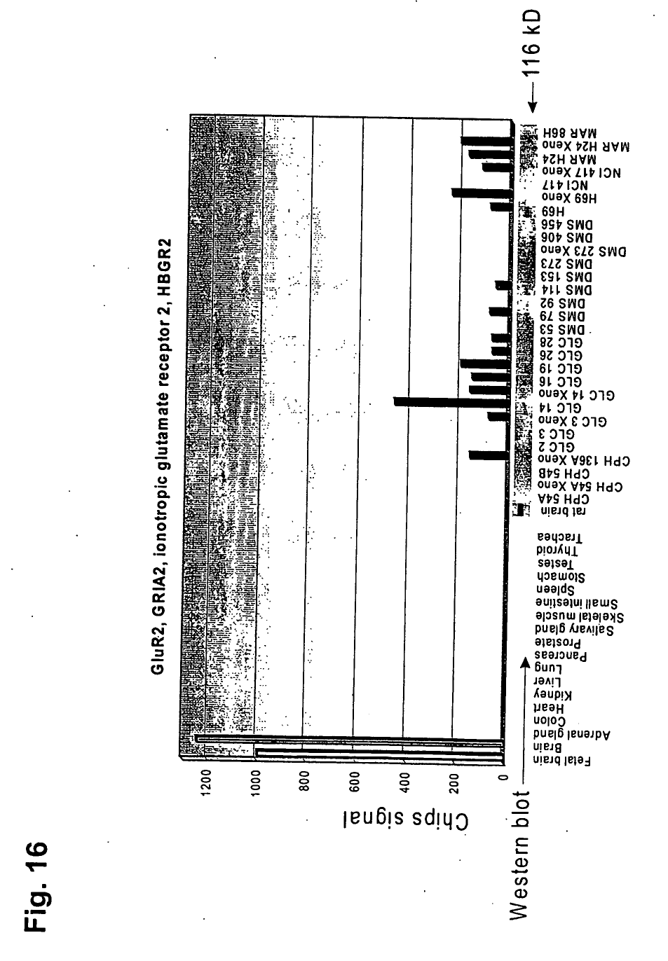

An especially preferred cell surface molecule according to the present invention is GRIA2 (Ionotropic glutamate receptor 2, GLUR2, GLURB, HBGR2, AMPA 2). GRIA2 is expressed in all tested SCLC and in the brain. GRIA2 is a highly specific SCLC receptor outside the brain. Expression of GRIA2 has for example been demonstrated by Chips analysis, RT-PCR and Western blotting (see example 1, FIG. 12A and FIG. 16).

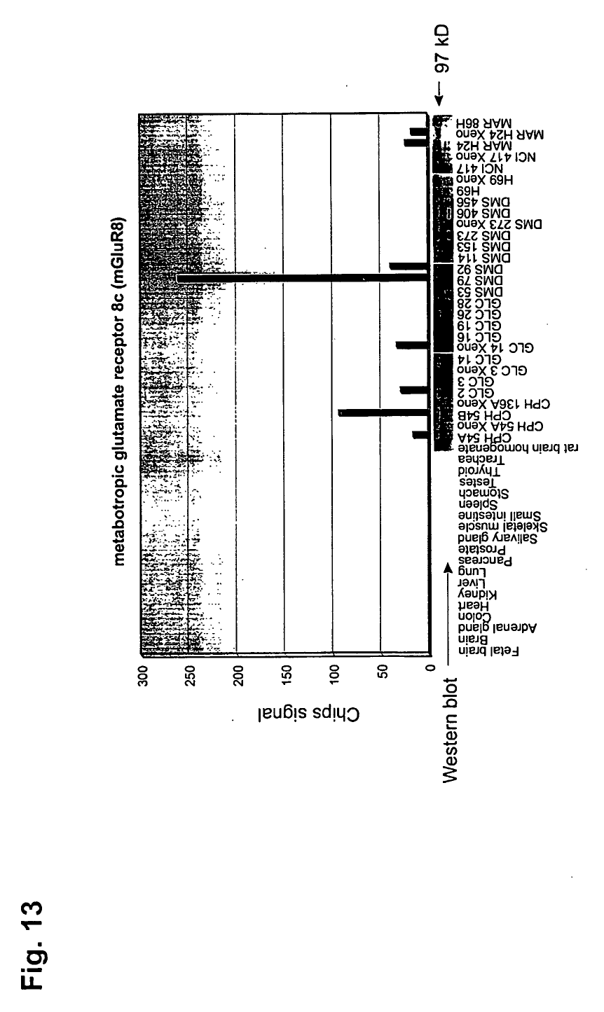

Another preferred cell surface molecule according to the present invention is GRM8 (metabotropic glutamate 8 receptor, GLUR8, mGlu8, GPRC1H). GRM8 is highly specifically expressed in SCLC except for the brain and is expressed in most SCLC. The expression of GRM8 has been demonstrated by for example Chips analysis, RT-PCR and western blotting (see example 1 and FIG. 13). Binding partners capable of associating with GRM8 are described herein below.

Yet another preferred cell surface molecule according to the invention is ITGAV (Integrin subunit αv, vitronectin receptor, CD 51). ITGAV is highly expressed by SCLC. Expression has been demonstrated by for example CHIPS analysis and RT-PCR (see example 1 and FIG. 12B)

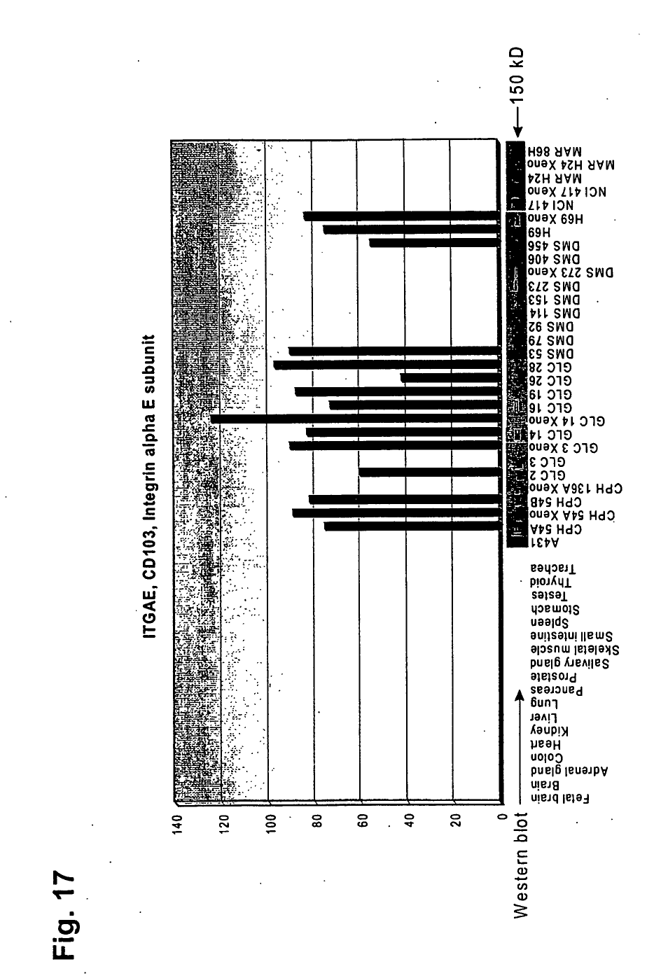

Yet another preferred cell surface molecule according to the present invention is ITGAE (integrin αE subunit-precursor, human mucosal lymphocyte-1′antigen, CD 103). ITGAE is expressed by all SCLC tested and ITGAE is highly specifically expressed in SCLC. Expression has been demonstrated by for example CHIPS analysis and western blotting (see example 1 and FIG. 17).

Yet another preferred cell surface molecule according to the present invention is GPR49 (orphan G protein-coupled receptor 67, GPR67, HG38).

Yet another preferred cell surface molecule according to the present invention is TMEFF1 (transmembrane protein with EGF-like and two follastatin-like domains).

However, the present invention is also directed towards cell surface molecules which comprises fragments, which are encoded by fragments of the nucleotide sequences given in table 2. In one preferred embodiment, the present invention is directed towards cell surface molecules encoded by splice variants of these sequences, which are encoded by the same gene. Splice variants of cell surface molecules outlined in table 2 may encode a polypeptide sequence which share fragments with said cell surface molecules, however splice variants may take advantage of an alternative reading frame, so that although the products of the two splice variants are encoded by nucleotide sequences that share common fragments, the polypeptide sequences may not be related.

Furthermore, the present invention is directed to fragments of the nucleotide sequences encoding cell surface molecules according to table 2. In particular, binding partners according to the present invention (se herein below) may associate with products of only one or more fragments of a cell surface molecule according to the present invention, but preferably not with all fragments of a cell surface molecule. Accordingly, it is possible to use fragments of the cell surface molecules to identify potential binding partners (see herein below).

For example such fragments comprise the 5′ half of the sequence or the 3′ half of the sequences. Furthermore, the fragments may comprise part of the 5′ half or part of the 3′ half of the sequences. Preferably, such fragments are shorter than 5000 bp, such as shorter than 4000 bp, for example shorter than 3000 bp, such as shorter than 2500 bp, for example shorter than 2000 bp, such as shorter than 1750 bp, such as shorter than 1500 bp, for example shorter than 1250 bp, such as shorter than 1000 bp, for example shorter than 900 bp, such as shorter than 800 bp, for example shorter than 700 bp, such as shorter than 600 bp, for example shorter than 500 bp, such as shorter than 400 bp, for example shorter than 300 bp, such as shorter than 200 bp, for example shorter than 100 bp, such as shorter than 75 bp, for example shorter than 50 bp, such as shorter than 40 bp, for example shorter than 30 bp, such as shorter than 25 bp, for example shorter than 20 bp, such as shorter than 18 bp.

Such fragments may be internal fragments or they may be comprise the 5′ or the 3′ terminal.

In one preferred embodiment of the present invention the fragments comprise a plurality of building blocks of a predetermined length and wherein the building blocks are linked so that the fragments are identical to part of a native gene sequence, preferably the sequences outlined in table 2. Accordingly, fragments may comprise a plurality of building blocks of the predetermined length and a predetermined starting point.

Building blocks are nucleic acid sequences, which have a predetermined length and starting point, so that the first building block starts at a given position in the nucleic acid sequence and the subsequent building blocks starts at the position following the position where the previous building block stops.

Preferably, the building blocks are derived from any of the cDNA/DNA sequences mentioned in table 2.

Each building block is preferably shorter than 1000 bp, for example shorter than 900 bp, such as shorter than 800 bp, for example shorter than 700 bp, such as shorter than 600 bp, for example shorter than. 500 bp, such as shorter than 400 bp, for example shorter than 300 bp, such as shorter than 200 bp, for example shorter than 100 bp, such as shorter than 75 bp, for example shorter than 50 bp, such as shorter than 40 bp, for example shorter than 30 bp, such as shorter than 25 bp, for example shorter than 20 bp, such as shorter than 18 bp. In one embodiment the building block is around 18 bp.

The building blocks may start at position 1, such as position 2, for example position 3, such as position 4, for example position 5, such as position 6, for example position 7, such as position 8, for example, position 9, such as position 10, for example position 11, such as position 12, for example position 13, such as position 14, for example position 15, such as position 16, for example position 17, such as position 18, for example, position 19, such as position 20, such as any of the positions 20 to 100, for example any of the position 100 of any of the sequences outlined in table 2.

The fragments preferably comprise a plurality of building blocks, such as 2, for example 3, such as 4, for example 5, such as from 5 to 10, for example from 10 to 20, such as from 20 to 30, for example from 30 to 40, such as from 40 to 50, for example from 50 to 75, such as from 75 to 100, for example more than 100 building blocks.

In one embodiment the fragments comprise building blocks which are 100 base pairs long and which start at position 1.

Furthermore, fragments of cell surface molecules according to the present invention may be chimeric fragments, such chimeric fragments comprise more than one fragments which are not associated with each other according to the sequences outlined in table 2. Such chimeric fragments may comprise fragments from the same cell surface molecule or they may contain fragments from more than one cell surface molecule according to the invention.

Furthermore, the present invention is directed to fragments of the polypeptides sequences of cell surface molecules according to table 2. In particular, binding partners according to the present invention (se herein below) may associate with only one or more fragments of a cell surface molecule according to the present invention, but preferably not with all fragments of a cell surface molecule. Accordingly, it is possible to use fragments of the cell surface molecules to identify potential binding 10′ partners (see herein below).

Fragments of polypeptide sequences may be shorter than 3000 amino acids, such as shorter than 2500 amino acids, for example shorter than 2000 amino acids, such as shorter than 1750 amino acids, such as shorter than 1500 amino acids, for example shorter than 1250 amino acids, such as shorter than 1000 amino acids, for example shorter than 900 amino acids, such as shorter than 800 amino acids, for example shorter than 700 amino acids, such as shorter than 600 amino acids, for example shorter than 500 amino acids, such as shorter than 400 amino acids, for example shorter than 300 amino acids, such as shorter than 200 amino acids, for example shorter than 100 amino acids, such as shorter than 75 amino acids, for example shorter than 50 amino acids, such as shorter than 40 amino acids, for example shorter than 30 amino acids, such as shorter than 25 amino acids, for example shorter than 20 amino acids, such as shorter than 15 amino acids, for example shorter than 10 amino acids.

Preferably, the fragments are fragments of polypeptide sequences SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40, SEQ ID NO. 43, SEQ ID NO. 45, SEQ ID NO. 47, SEQ ID NO. 49, SEQ ID NO. 51, SEQ ID NO. 53, SEQ ID NO. 55, SEQ ID NO. 57, SEQ ID NO. 59, SEQ ID NO. 61, SEQ ID NO. 63, SEQ ID NO. 65, SEQ ID NO. 67, SEQ ID NO. 69, SEQ ID NO. 71, SEQ ID NO. 73, SEQ ID NO. 75, SEQ ID NO. 77, SEQ ID NO. 79, SEQ ID NO. 81, SEQ ID NO. 83, SEQ ID NO. 85, SEQ ID NO. 87, SEQ ID NO. 89, SEQ ID NO. 91, SEQ ID NO. 93, SEQ ID NO. 95, SEQ ID NO. 97, SEQ ID NO. 99, SEQ ID NO. 101, SEQ ID NO. 103, SEQ ID NO. 105, SEQ ID NO. 107, SEQ ID NO. 109, SEQ ID NO. 111, SEQ ID NO. 113, SEQ ID NO. 115, SEQ ID NO. 117, SEQ ID NO. 119, SEQ ID NO. 120, SEQ ID NO. 121, SEQ ID NO. 122, SEQ ID NO. 123, SEQ ID NO. 124 SEQ ID NO. 125, SEQ ID NO. 126, SEQ ID NO. 127, SEQ ID NO. 129, SEQ ID NO. 131, SEQ ID NO. 133, SEQ ID NO. 135, SEQ ID NO. 137, SEQ ID NO. 139, SEQ ID NO. 141, SEQ ID NO. 143, SEQ ID NO. 145, SEQ ID NO. 147, SEQ ID NO. 149, SEQ ID NO. 151, SEQ ID NO. 153, SEQ ID NO. 155, SEQ ID NO. 157, SEQ ID NO. 159, SEQ ID NO. 161, SEQ ID NO. 163, SEQ ID NO. 165, SEQ ID NO. 167, SEQ ID NO. 169, SEQ ID NO. 171, SEQ ID NO. 173, SEQ ID NO. 175, SEQ ID NO. 177, SEQ ID NO. 179, SEQ ID NO. 181, SEQ ID NO. 183, SEQ ID NO. 185, SEQ ID NO. 187, SEQ ID NO. 189, SEQ ID NO. 191, SEQ ID NO. 193, SEQ ID NO. 195, SEQ ID NO. 197, SEQ ID NO. 199, SEQ ID NO. 201, SEQ ID NO. 203, SEQ ID NO. 205, SEQ ID NO. 207, SEQ ID NO. 208, SEQ ID NO. 209, SEQ ID NO. 210, SEQ ID NO. 211, SEQ ID NO. 212, SEQ ID NO. 213, SEQ-ID NO. 214, SEQ ID NO. 215, SEQ ID NO. 216, SEQ ID NO. 217, SEQ ID NO. 218, SEQ ID NO. 219, SEQ ID NO. 220, SEQ ID NO. 221, SEQ ID NO. 222, SEQ ID NO. 224, SEQ ID NO. 226, SEQ ID NO. 228, SEQ ID NO. 230, SEQ ID NO. 232, SEQ ID NO. 234, SEQ ID NO. 236, SEQ ID NO. 238, SEQ ID NO. 240, SEQ ID NO. 242 and SEQ ID NO. 244, SEQ ID NO. 246, SEQ ID NO. 248, SEQ ID NO. 250, SEQ ID NO. 252, SEQ ID NO. 254, SEQ ID NO. 256, SEQ ID NO. 258, SEQ ID NO. 260, SEQ ID NO. 262, SEQ ID NO. 264, SEQ ID NO. 266, SEQ ID NO. 268, SEQ ID NO. 270, SEQ ID NO. 272, SEQ ID NO. 274, SEQ ID NO. 276, SEQ ID NO. 278, SEQ ID NO. 280, SEQ ID NO. 282 SEQ ID NO. 284, SEQ ID NO. 286, SEQ ID NO. 288, SEQ ID NO. 290, SEQ ID NO. 292, SEQ ID NO. 294, SEQ ID NO. 296, SEQ ID NO. 298, SEQ ID NO. 300, SEQ ID NO. 302 and SEQ ID NO. 304. Furthermore, functional homologues of the fragments of the polypeptide sequences may also be comprised within cell surface molecules of the present invention, or the cell surface molecules may consist of functional homologues of the fragments of the polypeptide sequences. Functional homologues are defined herein below.

Particularly preferred fragments of the cell surface molecules according to the present invention are fragments that comprises one or more extracellular domains of the cell surface molecules. Additionally, fragments which comprise parts of extracellular domains are also preferred fragments within the scope of the present invention. Most preferably, the fragments of the cell surface molecules according to the present invention are fragments that comprise one or more extracellular domains and which are capable of internalising a binding partner having affinity for said fragment.

In one embodiment the fragments comprise an extracellular domain or fragments thereof or derivatives thereof, wherein said extracellular domain may be selected from the group consisting of polypeptide sequences, which are encoded by the nucleotide sequences SEQ ID NO 3 nucleotide 1014 to 2450, SEQ ID NO 15 nucleotide 216 to 3179, SEQ ID NO. 31 nucleotide 147 to 2999, SEQ ID NQ 52 nucleotide 419 to 4120, SEQ ID NO 66 nucleotide 268 to 7059, SEQ ID NO 82 nucleotide 235 to 2094, SEQ ID N0104 nucleotide 160 to 663, SEQ ID NO 204 nucleotide 301 to 2250, SEQ ID NO 229 nucleotide 130 to 2880, SEQ ID NO 281 nucleotide 569 to 2152, SEQ ID NO 283 nucleotide 585 to 1901, SEQ ID NO 291 nucleotide 121 to 2836 and SEQ ID NO 293 nucleotide 259 to 2127.

The cell surface molecule and the fragments of cell surface molecules as outlined herein above, may furthermore comprise posttranslational modifications. Examples of posttranslational modifications are phosphorylations, glycosylation, acetylations, methylation, sulfatation, polysialylation, farnesylation, myristoylation or palmitylation.

Functional homologues of the cell surface molecules outlined in table 2 are also contained within the present invention. SEQ ID NO of polypeptide sequences encoding preferred cell surface molecules according to the present invention are also given in table 2. Functional homologues of cell surface molecules according to the present invention are cell surface molecules which can associate with the binding: partners according to the present invention and which preferably can internalise said binding partners.

Promoters

Promoters within the scope of the present invention are first nucleic acid sequences, which are capable of directing expression of second nucleic acid sequences operably linked thereto. Such first nucleic acid sequences are normally found upstream on the chromosome of nucleic acid sequences that may be transcribed.

In one embodiment, preferably a first nucleic acid sequence operably linked to a second nucleic acid sequence comprise at least 15,000 base pairs upstream of the translation start codon of said second nucleic acid sequence on the chromosome.

However, the first nucleic acid sequence operably linked to a second nucleic acid sequence may also comprise at least 12.500, such as at least 10.000, for example at least 8,000, such as at least 6,000, such as at least 5,000, such as at least 4,000, such as at least 3,000, for example at least 2,500, such as at least 2,000, such as at least 1,500, such as at least 1,000, for example at least 500, such as at least 400, for example at least 300, such as at least 200, for example at least 150, such as at least 100, for example at least 50, such as at least 25, for example at least 10 base pairs upstream of the translation start codon of said second nucleic acid sequence on the chromosome, or a fragment of any such sequences capable of directing gene expression.

Furthermore, in another embodiment the first nucleic acid sequence operably linked to a second nucleic acid sequence preferably comprises up to 10, such as up to 100, such as up to 500, for example up to 1000, such as up to 2500, for example up to 5000 base pairs upstream of the translation start codon of said second nucleic acid sequence on the chromosome, or a fragment thereof capable of directing gene expression.

It is also within the scope of the present invention, that the first nucleic acid sequence operably linked to a second nucleic acid sequence may comprise one or more intron sequences or fragments thereof found upstream of the translation start codon of said second nucleic acid sequence on the chromosome. Furthermore, the first nucleic acid sequence operably linked to a second nucleic acid sequence may comprise one or more intron sequences or fragments thereof found downstream of the translation start codon of said second nucleic acid sequence on the chromosome.

The first nucleic acid sequence operably, linked to a second nucleic acid sequence may furthermore comprise an enhancer sequence located more than 15,000 base pairs upstream or downstream from the translation start codon of said second nucleic acid sequence on the chromosome.

The above mentioned first nucleic acid sequence operably linked to a second nucleic acid sequence may also comprise deletions and/or additions of nucleic acids. Deletions and/or additions may be internal or they may be at the end of the nucleic acid sequence.

Accordingly, from a first nucleic acid sequence, which for example comprise up to 1000, such as up to 2500, for example up to 5000, such as up to 7500, such as up to 10,000, for example up to 15,000, such as up to 20,000 base pairs upstream from the translation start codon of a second nucleic acid sequence on the chromosome, at least 10 internal bp, such as at least 25 internal bp, for example at least 50 internal bp, such as at least 100 internal bp, for example 200 internal bp, such as at least 300 internal bp, for example at least 400 internal bp, such as at least 500 internal bp, for example at least 750 internal bp, such as at least 1000 internal bp, for example at least 1250, such as at least 1500, for example at least 1750, such as at least 2000, for example at least 2500, such as at least 3000, for example at least 3500, such as at least 4000, for example at least 4500 internal base pairs may be deleted.

Specific nucleic acid sequences may be more favourable to delete than others. For example sequences that suppress expression or sequences that do not alter expression of second nucleic acid sequences operably linked thereto may be deleted. Accordingly, the present invention also encompass first nucleic acid sequence operably linked to a second nucleic acid sequence comprising up to 1000, such as up to 2500, for example up to 5000, such as up to 7500, such as up to 10,000, for example up to 15,000, such as up to 20,000 base pairs upstream from the translation start codon of a second nucleic acid sequence on the chromosome, from which at least one silencer has been deleted.

It is also possible that the first nucleic acid sequence comprises more than one deletion, such as 2, for example 3, such as 4, for example 5, such as more than 5 deletions.

Furthermore, from a first nucleic acid sequence, which for example comprise up to 20,000, for example up to 15,000, such as up to 10,000, for example up to 7500, for example up to 5000, such as up to 2500, for example up to 1000 base pairs upstream from the translation start codon of a second nucleic acid sequence on the chromosome, at least 10, such as at least 25, for example at least 50, such as at least 100, for example 200, such as at least 300, for example at least 400, such as at least 500, for example at least 750, such as at least 1000, for example at least 1250, such as at least 1500, for example at least 1750, such as at least 2000, for example at least 2500, such as at least 3000, for example at least 3500, such as at least 4000, for example at least 4500 base pairs may be deleted from either one or the other end.

Additions of nucleic acid sequences may be done at the end or internally. First nucleic acid sequences may comprise more than one addition, for example 2, such as 3, for example 4, such as 5, for example more than 5 additions. It may be addition of at least 10, such as at least 25, for example at least 50, such as at least 100, for example 200, such as at least 300, for example at least 400, such as at least 500, for example at least 750, such as at least 1000, for example at least 1250, such as at least 1500, for example at least 1750, such as at least 2000, for example at least 2500, such as at least 3000, for example at least 3500, such as at least 4000, for example at least 4500, such as more than 4500 base pairs.

For example it is possible to add nucleic acid sequences that alter the function of the first nucleic acid sequence. For example nucleic acid sequences which ate recognised by specific transcription factors may be added. For example nucleic acid sequences that are recognised by nuclear steroid hormone receptors

Examples of preferred first nucleic acid sequences are given in table 3 and 4. Functional homologues as described herein below of these nucleic acid sequences as well as deletion and/or addition mutants as described herein above are also comprised with in the present invention.

TABLE 3
First nucleic acid sequences indicating accession no.
in the Blast database Version: May 10, 2001
(http://www.ncbi.nlm.nih.gov/genome/seq/page.cgi?F=HsBlast.html&&ORG=Hs)

| | | Chromo- | | |
|---|---|---|---|---|
| No | Acc. No. | some | Region | Bases |
| pro4 | J05614 | 20 | NT_011387.3 | 4.073.925-4.125.777 |
| | | | − | |
| pro12 | AF059531 | 11 | NT_009107.3 | 16.582.000-16.639.000 |
| | | | + | |
| pro17 | AA913812 | 1 | NT_004705.3 | 145.236.000-145.282.000 |
| | | | + | |
| pro18 | W25866 | 1 | NT_004705.3 | 145.240.856-145.281.103 |
| | | | + | |
| pro28 | W74442 | 15 | NT_010356.3 | 87.186.215-87.226.899 |
| | | | − | |
| pro37 | U63743 | 1 | NT_004808.3 | 73.601.528-73.642.295 |
| | | | − | |
| pro49 | AA203476 | 4 | NT_006291.3 | 46.964.650-47.005.368 |
| | | | + | |
| | | 8 | NT_008166.3 | 66.825.152-66.865.799 |
| | | | + | |
| pro53 | M15205 | 17 | NT_024874.3 | 78.209.000-78.266.000 |
| | | | ? | |
| pro55 | Z95118 | 6 | NT_007577 | |
| pro62 | AL050306 | X | NT_011553 | |
| | | 11 | NT_025842 | |
| pro73 | U18271 | 12 | NT_009681.3 | 103.894.000-103.940.000 |
| | | | + | |
| pro74 | U03735 | X | NT_011534.1 | 138.253.000-138.770.000 |
| | | | + | |
| pro76 | U89387 | 2 | NT_005409.3 | 130.026.000-130.083.000 |
| | | | − | |
| pro77 | U10689 | X | NT_011726.3 | 137.038.000-137.108.000 |
| | | | ? | |
| pro83 | AI553745 | 5 | NT_023270.3 | 177.081.000-177.128.000 |
| | | | − | |
| pro86 | L18877 | X | NT_011534.1 | 138.233.000-138.771.000 |
| | | | + | |
| pro87 | U10691 | X | NT_011534.1 | 138.233.000-138.770.000 |
| | | | + | |
| pro89 | L18920 | X | NT_011534.1 | 138.233.000-138.770.000 |
| | | | + | |
| pro91 | N23137 | 12 | NT_009464.3 | 132.284.168-132.324.678 |
| | | | + | |
| pro93 | W28479 | 13 | NT_024495.3 | 87.871.068-87.891.259 |
| | | | + | |
| pro97 | AF053305 | 6 | NT_007592.3 | 37.167.152-37.207.246 |
| | | | + | |
| | | 12 | NT_009782.3 | 53.429.369-53.469.399 |
| | | | + | |
| pro103 | U77949 | 17 | NT_024901.3 | 40.905.000-40.968.000 |
| | | | − | |
| pro112 | AL021546 | 12 | NT_009775 | |
| pro120 | M31315 | 5 | NT_023188.3 | 176.642.000-176.692.000 |
| | | | ? | |
| pro121 | D26488 | 1 | NT_004680.3 | 214.431.387-214.471.483 |
| | | | + | |
| pro122 | AL021366 | 6 | NT_007592.3 | 31.000.000-38.000.000 |
| | | | + | |
| pro123 | N58115 | 2 | NT_005343.3 | 184.162.000-184.223.000 |
| | | | − | |
| pro129 | AL031427 | 1 | NT_004424 | |
| pro139 | Y13115 | 8 | NT_008176.3 | 39.029.000-39.073.000 |
| | | | − | |
| pro142 | AA151922 | 17 | NT_010711.3 | 73.365.739-73.406.421 |
| | | | − | |
| pro153 | U07563 | 9 | NT_008338 | |
| pro154 | W26762 | 1 | NT_019273.3 | 115.514.000-115.557.000 |
| | | | − | |
| pro156 | AI950382 | 17 | NT_010641.3 | 77.160.000-77.208.000 |
| | | | + | |
| pro161 | M21259 | 1 | NT_004662.3 | 175.106.661-175.147.391 |
| | | | + | |
| pro163 | U75285 | 17 | NT_024874 | |
| pro166 | AA926957 | 5 | NT_016864.3 | 52.120.104-52.140.918 |
| | | | + | |
| | | 17 | NT_010692.3 | 1.029.000-1.077.000 |
| | | | + | |
| pro171 | M90354 | 13 | NT_024495.3 | 89.323.836-89.365.575 |
| | | | + | |
| pro172 | AA143321 | 18 | NT_010934.3 | 35.186.000-35.241.000 |
| | | | − | |
| pro176 | AA181196 | 13 | NT_009799.3 | 49.703.000-49.752.000 |
| | | | + | |
| pro178 | AB002359 | 15 | NT_010364.3 | 19.619.949-19.660.316 |
| | | | − | |
| pro183 | U13695 | 2 | NT_022197.3 | 197.599.000-197.820.000 |
| | | | − | |
| pro184 | U65011 | 22 | NT_011520.5 | 18.956.000-19.011.000 |
| | | | + | |
| pro194 | AF091754 | 1 | NT_004734.3 | 233.039.886-233.081.072 |
| | | | + | |
| pro198 | AL049842 | 13 | NT_009910 | |
| pro202 | AA810792 | 12 | NT_009464.3 | 132.288.241-132.328.844 |
| | | | + | |
| pro210 | L37747 | 5 | NT_023326.1 | 122.981.845-123.022.988 |
| | | | − | |
| pro212 | Z36714 | 2 | NT_005370.3 | 212.774.647-212.814.782 |
| | | | − | |
| pro216 | X06745 | X | NT_011821.1 | 21.110.482-21.151.898 |
| | | | ? | |
| pro225 | T75292 | 4 | NT_006088.3 | 82.210.258-82.250.894 |
| | | | + | |
| pro228 | U38979 | 1 | NT_021930.3 | 97.904.873-97.945.550 |
| | | | + | |
| pro229 | AL031778 | 6 | NT_016968 | |
| | | | − | |
| pro230 | AA044787 | 5 | NT_006964.3 | 154.054.000-154.122.000 |
| | | | − | |
| pro232 | M77481 | X | NT_011534.1 | 138.236.000-138.766.000 |
| | | | + | |
| pro234 | AL021397 | 1 | NT_004668 | |
| pro239 | AA595596 | 1 | NT_004668 | |
| pro240 | W25874 | 4 | NT_006088.3 | 82.210.000-82.256.000 |
| | | | + | |
| pro241 | AC004774 | 7 | NT_007816 | |
| Pro245 | AF006010 | 8 | NT_007978.3 | 104.966.000-105.280.000 |
| | | | − | |
| pro254 | AA733050 | 1 | NT_004662.3 | 175.107.000-175.116.000 |
| | | | + | |
| pro260 | AF094481 | 3 | NT_005791.3 | 84.866.000-84.938.000 |
| | | | − | |
| pro268 | L16991 | 5 | NT_023126.3 | 20.814.448-20.854.499 |
| | | | − | |
| pro273 | AC004770 | 11 | NT_004686; 11q12 | 64224936-64225035 |
| pro280 | AI985964 | 21 | NT_011514.4 | 43.166.000-43.211.000 |
| | | | − | |
| pro284 | AA926959 | 5 | NT_006687.3 | 48.821.840-48.862.512 |
| | | | − | |
| pro292 | U28386 | 4 | NT_006109.2 | : 89.638.000-89.682.000 |
| | | | − | |
| pro299 | AI087268 | 6 | NT_007193.3 | 38.723.000-38.783.000 |
| | | | + | |
| pro302 | AF014837 | 14 | NT_019583.3 | 18.376.000-18.432.000 |
| | | | + | |
| pro303 | AI570572 | 22 | NT_011520.5 | 33.485.000-33.531.000 |
| | | | + | |
| pro304 | L17131 | 2 | NT_005428.3 | 76.252.000-76.295.000 |
| | | | − | |
| pro306 | AI740522 | 6 | NT_007234.3 | 128.840.581-128.881.528 |
| | | | − | |
| pro328 | M15796 | X | NT_011568.3 | 33.655.000-33.706.000 |
| | | | − | |
| | | 20 | NT_011387.3 | 4.068.000-4.115.000 |
| | | | − | |
| pro329 | D00596 | 18 | NT_011005.3 | 849.000.000-872.000.000 |
| | | | + | |
| pro331 | W27939 | 12 | NT_009785.3 | 49.701.000-49.745.000 |
| | | | ? | |
| pro338 | AA768912 | 10 | NT_008609.3 | 26.001.705-26.042.334 |
| | | | − | |
| pro341 | AA877215 | 1 | NT_019273.3 | 114.182.965-114.223.575 |
| | | | − | |
| | | 13 | NT_009952.3 | 98.753.071-98.793.770 |
| | | | + | |
| pro344 | X16277 | 2 | NT_005380.3 | 10.809.000-10.860.000 |
| | | | + | |
| pro347 | AI032612 | 1 | NT_019269.3 | 41.189.000-41.265.000 |
| | | | − | |
| pro348 | AI032612 | 12 | NT_024394.3 | 101.992.000-102.107.000 |
| | | | − | |
| pro352 | AI525633 | 12 | NT_009785.3 | 49.699.479-49.740.007 |
| | | | ? | |
| pro358 | N95406 | 7 | NT_007816.3 | 96.997.000-97.064.000 |
| | | | ? | |
| pro361 | AA255502 | 6 | NT_007592.3 | 28.000.000-32.000.000 |
| | | | + | |

TABLE 4
First nucleic acid sequences indicating accession no.
in the human genome browser, 12 Dec. 2000 draft assembly
of the human genome Human genome browser

| | | Chromo- | | |
|---|---|---|---|---|
| No | Acc. No. | some | Band | Bases |
| pro1 | M87338 | 7 | 7q11.22 | 67380990-67396543 |
| pro2 | U73379 | 20 | 20q13.11 | 46319851-46338917 |
| pro3 | X05360 | 10 | 10q21.2 | 64194062-64211044 |
| pro5 | M25753 | 5 | 5q23.2 | 137604120-137622654 |
| | | 5 | 5q22.2 | 121662724-121679510 |
| | | 5 | 5q13.1 | 73649568-73666303 |
| pro6 | M74558 | 1 | 1p33 | 52577823-52592535 |
| pro7 | D38073 | 6 | 6p12.2 | 56968405-56986831 |
| pro8 | AF015254 | 17 | 17p12 | 9089031-9106502 |
| pro9 | J03626 | 3 | 3q21.2 | 135784683-135799798 |
| pro10 | U74612 | 12 | 12p13.33 | 3074066-3089514 |
| pro13 | D63880 | 12 | 12p13.31 | 6903002-6917288 |
| pro14 | D14657 | 15 | 15q22.2 | 60215632-60231472 |
| pro15 | AB024704 | 20 | 20q11.21 | 31872458-31887490 |
| pro16 | AI375913 | 17 | 17q21.2 | 42673095-42689004 |
| | (J04088) | | | |
| pro19 | U37426 | 10 | 10q23.33 | 99600001-99623479 |
| pro20 | AF098162 | 12 | 12q13.3 | 58470993-58506578 |
| pro21 | X74330 | 12 | 12q13.3 | 58801599-58817157 |
| pro22 | L47276 | 17 | 17q21.2 | 42703746-42719695 |
| pro23 | L25876 | 14 | 14q22.2 | 50958791-50974988 |
| pro24 | U65410 | 4 | 4q26 | 126153161-126168416 |
| pro25 | X13293 | 20 | 20q12 | 44147604-44164046 |
| pro26 | X51688 | 4 | 4q26 | 128042847-128058218 |
| pro27 | AL080146 | 15 | 15q22.1 | 55104198-55120101 |
| pro29 | D88357 | 10 | 10q21.2 | 64199199-64214445 |
| pro30 | D26361 | 1 | 1q32.1 | 224035414-224051265 |
| pro31 | D14678 | 6 | 6p21.31 | 36374334-36389597 |
| pro32 | AF011468 | 20 | 20q13.2 | 36644429-36659811 |
| | | | | 58763968-58779170 |
| pro34 | AB019987 | 3 | 3q25.33 | 175620714-175635845 |
| pro36 | AF053306 | 15 | 15q15.1 | 35625667-35641196 |
| pro38 | AF032862 | 5 | 5q34 | 178469476-178486752 |
| pro39 | U01038 | 16 | 16p21.1 | 28930379-28945875 |
| pro40 | D13633 | 14 | 14q22.3 | 29043898-29059035 |
| | | | | 51960394-51975921 |
| pro41 | AJ000186 | 4 | 4q26 | 126153146-126168525 |
| pro42 | D26018 | 11 | 11q13.4 | 79445361-79460511 |
| pro43 | X02308 | 18 | 18p11.32 | 931580-947280 |
| pro44 | AF016371 | 1 | 1p34.1 | 47366616-47382271 |
| pro45 | U05340 | 1 | 1p34.1 | 48138801-48153800 |
| pro46 | D80008 | 20 | 20p11.21 | 27258467-27274295 |
| pro47 | AB000449 | 14 | 14q32.2 | 96123633-96139456 |
| pro48 | AF017790 | 18 | 18p11.32 | 2615001-2630207 |
| pro50 | X51688 | 4 | 4q26 | 128041767-128057590 |
| pro51 | AF081280 | 10 | 10q24.32 | 109292185-109308954 |
| pro52 | M86699 | 6 | 6q14.1 | 85882001-85897200 |
| pro54 | X74794 | 8 | 8q11.22 | 50261007-50276309 |
| pro56 | AB006624 | 12 | 12q13.3 | 59174501-59189500 |
| pro57 | L38933 | 17 | 17q21.2 | 45056497-45072182 |
| pro58 | AB018334 | 5 | 5p13.2 | 40319301-40338000 |
| pro59 | AF091433 | 8 | 8q22.1 | 98382913-98396920 |
| pro60 | Z29066 | 1 | 1q32.3 | 237405001-237420000 |
| pro61 | D79988 | 12 | 12q24.31 | 133269001-133284000 |
| | | | | 133230501-133245500 |
| pro63 | AL050151 | 8 | 8q24.3 | 143930884-143952036 |
| pro64 | D79997 | 9 | 9p13.2 | 39124660-39148000 |
| pro65 | L07541 | 13 | 13q13.1 | 32415001-32433000 |
| pro66 | U37139 | 1 | 1p31.3 | 71792205-71807237 |
| pro67 | Y00272 | 10 | 10q21.2 | 64194599-64211207 |
| pro68 | D90070 | 18 | 18q21.32 | 62061499-62077998 |
| pro69 | U14518 | 2 | 2p23.3 | 26821351-26836883 |
| pro70 | X87843 | 14 | 14q23.1 | 57525402-57541401 |
| pro71 | U26727 | 9 | 9p21.3 | 23824001-23840000 |
| pro72 | X52142 | 1 | 1q42.12 | 251569001-251584900 |
| pro75 | L48692 | 2 | 2p13.3 | 69512098-69527598 |
| pro78 | L31801 | 1 | 1p13.2 | 124826201-124843002 |
| | | | | 124490069-124506743 |
| pro79 | U15552 | 1 | 1p22.1 | 105019659-105034740 |
| pro80 | AF039652 | 17 | 17p11.2 | 20094899-20110488 |
| pro81 | U79266 | 11 | 11q13.2 | 67334001-67349000 |
| pro82 | X92106 | 17 | 17q11.2 | 31700001-31715000 |
| pro84 | L07540 | 12 | 12q24.23 | 126266001-126283000 |
| pro85 | U76638 | 2 | 2q35 | 219994976-220010675 |
| pro88 | L34600 | 2 | 2p16.1 | 56591445-56606916 |
| pro90 | D83781 | 11 | 11p11.2 | 48298842-48316506 |
| pro92 | X59618 | X | Xp11.3 | 41625001-41640000 |
| pro94 | U73704 | 1 | 1p22.1 | 102963001-102978500 |
| pro95 | D50923 | 1 | 1q42.13 | 256927030-256942117 |
| pro96 | M85085 | X | Xq22.1 | 100331001-100346000 |
| pro98 | AF025840 | 14 | 14q21.3 | 46340081-46355371 |
| pro99 | AF029670 | 17 | 17q23.2 | 63449001-63464000 |
| pro100 | AF070559 | 13 | 13q33.1 | 106141001-106156500 |
| pro101 | S78085 | 6 | 6q27 | 180902275-180917523 |
| pro104 | U39817 | 15 | 15q26.1 | 87983001-87998000 |
| pro105 | L23959 | 13 | 13q34 | 116658001-116673000 |
| pro106 | X77743 | 2 | 2q24.1 | 160044918-160062017 |
| pro107 | U10564 | 11 | 11p15.3 | 9127192-9142201 |
| pro108 | AB028069 | 7 | 7q21.12 | 88435001-88450000 |
| pro109 | AF053977 | 5 | 5q31.1 | 150402501-150422204 |
| pro110 | U09087 | 12 | 12q23.1 | 104568858-104585221 |
| pro111 | AF073362 | 11 | 11q21 | 101407914-101423315 |
| pro113 | L49054 | 3 | 3q25.32 | 173904528-173934748 |
| pro114 | X04327 | 7 | 7q33 | 139307570-139325937 |
| pra115 | AF074723 | 14 | 14q24.2 | 68654211-68669417 |
| pro116 | AF091092 | 1 | 1p32.3 | 58583305-58598434 |
| pro117 | AF058918 | 19 | 19q13.43 | 70037301-70052300 |
| pro118 | AF025441 | 15 | 15q15.1 | 36875108-36890661 |
| pro119 | AJ006591 | 4 | 4q31.1 | 148771298-148786657 |
| pro124 | L36529 | 18 | 18p11.32 | 254219-270019 |
| pro125 | X65550 | 10 | 10q26.2 | 138240294-138255077 |
| pro126 | M34065 | 5 | 5q31.2 | 150756331-150771379 |
| pro127 | L02547 | 20 | 20q13.2 | 58756755-58772000 |
| pro128 | U00238 | 4 | 4q12 | 59266501-59283500 |
| pro130 | L42450 | 2 | 2q31.1 | 176199115-176214588 |
| pro131 | X98253 | X | Xq24 | 122031634-122047863 |
| pro133 | U58970 | 20 | 20q12 | 45447501-45463000 |
| pro134 | AF077953 | 18 | 18q21.1 | 47833501-47849000 |
| pro135 | J00140 | 5 | 5q14.1 | 86483185-86498573 |
| | | 18 | 18q11.2 | 25676581-25692180 |
| pro136 | M74093 | 19 | 19q12 | 34192227-34207465 |
| pro137 | AF029669 | 17 | 17q23.1 | 63211106-63252789 |
| pro138 | AL050019 | 1 | 1p36.33 | 52302-67336 |
| pro140 | M68520 | 12 | 12q13.2 | 57983277-57998212 |
| pro141 | Y13467 | 17 | 17q12 | 41583543-41598142 |
| pro143 | U64805 | 17 | 17q21.31 | 45619001-45639000 |
| pro144 | U78082 | 14 | 14q24.2 | 68653184-68668183 |
| pro145 | Y15164 | X | Xp22.22 | 13061501-13076500 |
| pro146 | AF008442 | 6 | 6p21.1 | 47871392-47886807 |
| pro147 | X16901 | 13 | 13q14.13 | 43972520-43990027 |
| pro148 | U06632 | 17 | 17q23.1 | 61863000-61878839 |
| pro149 | D26069 | 3 | 3q29 | 215253329-215270320 |
| pro150 | AF027150 | 14 | 14q21.1 | 35344279-35359894 |
| pro152 | D13413 | 3 | 3q29 | 211580067-211591811 |
| pro155 | L20320 | 5 | 5q13.1 | 73642669-73657817 |
| pro157 | AJ223728 | 22 | 22q11.21 | 16393966-16409005 |
| pro158 | AB023215 | 14 | 14q24.3 | 74118287-74135032 |
| pro159 | D32002 | 9 | 9q22.33 | 98867915-98884079 |
| pro160 | Y18046 | 6 | 6q27 | 177044740-177060727 |
| pro162 | U27459 | 2 | 2q33.2 | 206003518-206019965 |
| pro164 | D86322 | 4 | 4q31.1 | 147998675-148013849 |
| pro165 | Z46376 | 2 | 2p12 | 75788712-75806284 |
| pro167 | AF003540 | 11 | 11p15.5 | 2576355-2591604 |
| pro168 | D38553 | 2 | 2q11.1 | 94745291-94760990 |
| pro169 | M60725 | 17 | 17q23.2 | 64866487-64881241 |
| pro173 | L34673 | 3 | 3q24 | 162499055-162515000 |
| pro174 | U93867 | 1 | 1q21.1 | 162966980-162983139 |
| pro175 | U44754 | 14 | 14q23.1 | 58567756-58582473 |
| pro177 | AL050405 | X | Xq26.1 | 132444071-132459420 |
| pro179 | U18937 | 5 | 5q31.3 | 153395323-153411137 |
| pro180 | X76388 | 4 | 4q31.21 | 152639006-152654005 |
| pro181 | AF038662 | 3 | 3q13.32 | 129394716-129409918 |
| pro182 | AF042169 | 10 | 10q22.1 | 74034210-74049959 |
| pro185 | U07804 | 20 | 20q12 | 41507121-41523513 |
| pro187 | M97388 | 1 | 1p22.1 | 104042438-104058335 |
| pro189 | L35546 | 1 | 1p22.1 | 104986774-105006727 |
| pro191 | AB007962 | 1 | 1q21.3 | 170103257-170119230 |
| pro193 | AF006259 | 12 | 12p13.22 | 4781441-4796771 |
| pro195 | X78627 | 2 | 2q14.3 | 123494635-123511228 |
| pro196 | M62810 | 10 | 10q21.1 | 61677452-61693234 |
| pro197 | AL080116 | 6 | 6q15 | 94998657-95013656 |
| pro199 | U79256 | 10 | 10q24.1 | 104501013-104516982 |
| pro201 | AL080088 | 16 | 16p13.2 | 11860550-11875029 |
| pro203 | M27878 | 12 | 12q24.33 | 144471103-144484312 |
| pro204 | AF091090 | 1 | 1p22.2 | 99597586-99612101 |
| pro205 | AF067656 | 10 | 10q21.1 | 59448924-59463923 |
| pro206 | U03911 | 2 | 2p16.3 | 48415852-48431851 |
| pro207 | U61145 | 7 | 7q36.1 | 155082360-155098279 |
| pro208 | D78335 | 1 | 1q23.3 | 187227102-187243552 |
| pro209 | U30872 | 1 | 1q41 | 240503771-240519770 |
| pro211 | U97188 | 7 | 7p15.3 | 23230613-23248257 |
| | | 6 | 6q27 | 176760451-176776177 |
| pro213 | D38550 | 6 | 6p22.3 | 22232782-22247803 |
| pro215 | X15331 | X | Xq22.3 | 108009046-108024961 |
| pro217 | D87448 | 3 | 3q22.1 | 146018136-146033999 |
| pro219 | U31556 | 8 | 8q21.3 | 88467142-88482808 |
| pro220 | X66113 | 1 | 1p36.22 | 11562849-11578096 |
| pro221 | M93119 | 20 | 20p11.23 | 21882614-21898000 |
| pro222 | AB020670 | 18 | 18q23 | 83636442-83651891 |
| pro223 | AF000430 | 12 | 12p11.21 | 33646020-33661381 |
| pro224 | U73960 | 7 | 7p21.3 | 11988001-11983000 |
| pro226 | X76029 | 4 | 4q12 | 58426181-58442374 |
| pro227 | AF063020 | 9 | 9p22.3 | 16919890-16934979 |
| pro231 | AJ132440 | 1 | 1q32.1 | 226438005-226453537 |
| pro233 | AB024401 | 13 | 13q34 | 113595323-113611692 |
| pro235 | AL049266 | 2 | 2q32.1 | 190196543-190212071 |
| pro236 | AB014550 | 18 | 18p11.32 | 2784040-2799481 |
| pro237 | X78932 | 7 | 7q11.21 | 64013301-64028356 |
| pro238 | U22377 | 1 | 1p34.2 | 44683920-44699429 |
| pro242 | L16782 | 10 | 10q23.31 | 96533196-96548690 |
| pro243 | AJ001810 | 16 | 16q12.2 | 63707495-63722576 |
| pro244 | U16028 | 2 | 2q31.1 | 178830447-178845919 |
| pro246 | L08424 | 12 | 12q23.2 | 109949544-109964900 |
| pro247 | U07559 | 5 | 5q11.2 | 53964473-53980257 |
| pro248 | Y10043 | X | Xq28 | 155144719-155160264 |
| pro249 | S74445 | 15 | 15q24.2 | 74840231-74855495 |
| pro250 | U25165 | 3 | 3q26.33 | 196934501-196950372 |
| pro251 | U63336 | 6 | 6p21.33 | 33662019-33677856 |
| pro253 | U13022 | 7 | 7q35 | 149686107-149702331 |
| pro255 | AB019494 | 5 | 5p13.2 | 39907204-39922203 |
| pro256 | L07919 | 2 | 2q31.1 | 175756788-175772636 |
| pro257 | AB029006 | 2 | 2p22.3 | 32520144-32540169 |
| pro258 | AB028995 | 17 | 17q23.2 | 63475124-63497063 |
| pro259 | U62325 | 2 | 2q35 | 223126524-223141423 |
| pro262 | X54942 | 9 | 9q22.1 | 87899001-87915976 |
| pro263 | U10860 | 4 | 4p16.1 | 9058241-9073583 |
| pro264 | M87339 | 3 | 3q27.3 | 204899461-204915188 |
| pro267 | X59543 | 11 | 11p15.5 | 3340168-3358000 |
| pro269 | M76180 | 7 | 7p12.2 | 51709937-51727010 |
| pro270 | M92299 | 17 | 17q21.32 | 52711008-52726713 |
| pro271 | AB028021 | 20 | 20p11.21 | 25363017-25378980 |
| pro272 | Y16752 | 6 | 6p22.2 | 27692102-27707775 |
| pro275 | U96131 | 5 | 5p15.33 | 1133261-1148076 |
| pro277 | X84194 | 14 | 14q24.3 | 73530456-73545798 |
| pro278 | D82345 | X | Xq22.1 | 102209954-102227610 |
| pro279 | L36818 | 11 | 11q13.4 | 76825241-76843255 |
| pro282 | V00568 | 8 | 8q24.13 | 130952716-130971510 |
| pro283 | U87459 | X | Xq28 | 157900553-157917852 |
| pro285 | X16396 | 2 | 2p12 | 74933738-74953722 |
| pro286 | AF007140 | 19 | 19p13.2 | 10684785-10702574 |
| pro287 | AF053641 | 20 | 20q13.13 | 50999953-51020590 |
| pro289 | X55110 | 11 | 11p11.2 | 47010261-47026357 |
| pro290 | U84573 | 3 | 3q24 | 159499984-159525599 |
| | | X | Xq26.2 | 137568108-137581997 |
| pro291 | M97856 | 1 | 1p34.1 | 50925640-50946744 |
| pro293 | AB011173 | 1 | 1p36.12 | 25196653-25221848 |
| pro294 | U34994 | 3 | 3p24.3 | 25352447-25367957 |
| pro295 | Y18004 | X | Xp22.13 | 18909706-18961865 |
| pro296 | D78611 | 7 | 7q32.2 | 133415266-133433462 |
| pro297 | D55716 | 7 | 7q22.1 | 101656642-101674644 |
| pro298 | L19183 | 17 | 17q11.2 | 29841430-29862449 |
| pro300 | X00737 | 14 | 14q11.2 | 16658977-16678611 |
| pro301 | X14850 | 9 | 9q21.12 | 73041595-73057571 |
| pro305 | M63180 | 5 | 5p13.3 | 32613067-32631841 |
| pro307 | AB014458 | 1 | 1p31.3 | 70502659-70519495 |
| pro308 | L07493 | 7 | 7p22.1 | 6711371-6728698 |
| pro309 | AF041474 | 3 | 3q26.33 | 198844404-198867622 |
| pro310 | Y18418 | 3 | 3q21.3 | 139858490-139872261 |
| pro311 | M94362 | NA_random | | 1360953-1386439 |
| pro312 | U50939 | 16 | 16q22.1 | 75154638-75172659 |
| pro313 | D64110 | 21 | 21q11.2 | 15926421-15943327 |
| pro315 | M91670 | 17 | 17p11.2 | 17349263-17365467 |
| | | 10 | 10p11.21 | 38991035-39007459 |
| pro316 | X64229 | 6 | 6p22.3 | 19889797-19909681 |
| pro317 | X53793 | 4 | 4q12 | 59189280-59209671 |
| pro318 | AL080119 | 1 | 1p31.2 | 76612579-76631294 |
| pro319 | D28423 | 6 | 6p21.31 | 40691099-40706383 |
| pro320 | U35451 | 17 | 17q21.32 | 52107808-52124651 |
| pro321 | U94319 | 9 | 9p22.3 | 16952805-16970295 |
| pro322 | M30938 | 2 | 2q35 | 221329992-221351121 |
| pro323 | AF047473 | 10 | 10q26.12 | 132970590-132988540 |
| pro324 | L33930 | 6 | 6q21 | 114313634-114328131 |
| pro326 | X62534 | 4 | 4q34.1 | 182348719-182364558 |
| pro327 | D84557 | 2 | 2q22.1 | 138697866-138715350 |
| pro330 | D21063 | 3 | 3q21.3 | 139528312-139551423 |
| pro332 | U90426 | 19 | 19p13.13 | 15547854-15568398 |
| pro333 | D00762 | 14 | 14q23.1 | 55012125-55034336 |
| pro334 | U72066 | 18 | 18q11.2 | 22625608-22643453 |
| pro335 | M86737 | 11 | 11q12.1 | 57630355-57653499 |
| pro336 | D13627 | 21 | 21q21.3 | 27298411-27317280 |
| pro337 | X01060 | 3 | 3q29 | 216708325-216729920 |
| pro339 | AF039022 | 20 | 20q11.22 | 34329184-34345180 |
| pro340 | L27706 | 7 | 7p11.2 | 57350245-57365971 |
| pro346 | AF035316 | 6 | 6p25.2 | 3491115-3506631 |
| pro349 | U25182 | X | Xp22.12 | 22338422-22357291 |
| pro353 | X74262 | 1 | 1p35.1 | 35716074-35777340 |
| pro354 | J02645 | 14 | 14q23.3 | 64971951-64997045 |
| pro355 | M37583 | 4 | 4q24 | 104191912-104207386 |
| pro356 | U37689 | 3 | 3q27.2 | 202022093-202041189 |
| pno359 | U09510 | 7 | 7p14.3 | 30543349-30559412 |
| Pro362 | X04741 | 4 | 4p4 | 39926899-39927127 |

Preferably, the first nucleic acid sequences are selected from the group consisting of pro1, pro2, pro3, pro4, pro5, pro6, pro7, pro8, pro9, pro10, pro12, pro13, pro14, pro15, pro16, pro17, pro18, pro19, pro20, pro21, pro22, pro23, pro24, pro25, pro26, pro27, pro28, pro29, pro30, pro31, pro32, pro34, pro36, pro37, pro38, pro39, pro40, pro41, pro42, pro43, pro44, pro45, pro46, pro47, pro48, pro49, pro50, pro51, pro52, pro53, pro54, pro55, pro56, pro57, pro58, pro59, pro60, pro61, pro62, pro63, pro64, pro65, pro66, pro67, pro68, pro69, pro70, pro71, pro72, pro73, pro74, pro75, pro76, pro77, pro78, pro79, pro80, pro81, pro82, pro83, pro84, pro85, pro86, pro87, pro88, pro89, pro90, pro91, pro92, pro93, pro94, pro95, pro96, pro97, pro98, pro99, pro100, pro101, pro103, pro104, pro105, pro106, pro107, pro108, pro109, pro 110, pro111, pro112, pro113, pro114, pro15, pro116, pro117, pro118, pro119, pro120, pro121, pro122, pro123, pro124, pro125, pro126, pro127, pro128, pro129, pro130, pro131, pro133, pro134, pro135, pro136, pro137, pro138, pro139, pro140, pro141, pro142, pro143, pro144, pro145, pro146, pro147, pro148, pro149, pro150, pro152, pro153, pro154, pro155, pro156, pro157, pro158, pro159, pro160, pro161, pro162, pro163, pro164, pro165, pro166, pro167, pro168, pro169, pro171, pro172, pro173, pro174, pro175, pro176, pro177, pro178, pro179, pro180, pro181, pro182, pro183, pro184, pro185, pro187, pro189, pro191, pro193, pro194, pro195, pro196, pro197, pro198, pro199, pro201, pro202, pro203, pro204, pro205, pro206, pro207, pro208, pro209, pro210, pro211, pro212, pro213, pro215, pro216, pro217, pro219, pro220, pro221, pro222, pro223, pro224, pro225, pro226, pro227, pro228, pro229, pro230, pro231, pro232, pro233, pro234, pro235, pro236, pro237, pro238, pro239, pro240, pro241, pro242, pro243, pro244, pro245, pro246, pro247, pro248, pro249, pro250, pro251, pro253, pro254, pro255, pro256, pro257, pro258, pro259, pro260, pro262, pro263, pro264, pro267, pro268, pro269, pro270, pro271, pro272, pro273, pro275, pro277, pro278, pro279, pro280, pro282, pro283, pro284, pro285, pro286, pro287, pro289, pro290, pro291, pro292, pro293, pro294, pro295, pro296, pro297, pro298, pro299, pro300, pro301, pro302, pro303, pro304, pro305, pro306, pro307, pro308, pro309, pro310, pro311, pro312, pro313, pro315, pro316, pro317, pro318, pro319, pro320, pro321, pro322, pro323, pro324, pro326, pro327, pro328, pro329, pro330, pro331, pro332, pro333, pro334, pro335, pro336, pro337, pro338, pro339, pro340, pro341, pro344, pro346, pro347, pro348, pro349, pro352, pro353, pro354, pro355, pro356, pro358, pro359 and pro361.

More preferably, the first nucleic acid sequence is selected from the group consisting of pro1, pro2, pro3, pro4, pro5, pro6, pro7, pro8, pro9, pro10, pro12, pro13, pro14, pro15, pro16, pro17, pro18, pro19, pro21, pro22, pro23, pro24, pro25, pro26, pro27, pro28, pro29, pro30, pro31, pro32, pro34, pro36, pro37, pro38, pro39, pro40, pro41, pro42, pro43, pro44, pro45, pro46, pro47, pro48, pro49, pro50, pro51, pro52, pro53, pro54, pro56, pro58, pro59, pro62, pro64, pro65, pro66, pro68, pro69, pro70, pro71, pro72, pro73, pro74, pro75, pro77, pro78, pro81, pro82, pro85, pro86, pro87, pro89, pro90, pro92, pro98, pro103, pro105, pro108, pro120, pro125, pro128, pro130, pro133, pro135, pro136, pro137, pro157, pro184, pro205, pro206, pro207, pro209, pro210, pro211, pro212, pro216, pro217, pro219, pro221, pro227, pro231, pro233, pro241, pro246, pro248, pro249, pro253 and pro256.

Yet more preferably, the first nucleic acid sequences are selected from the group consisting of pro1, pro2, pro3, pro4, pro5, pro7, pro8, pro14, pro15, pro16, pro22, pro23, pro24, pro26, pro27, pro29, pro34, pro37, pro38, pro39, pro40, pro45, pro46, pro48, pro49, pro50, pro52, pro59, pro69, pro71, pro74, pro77, pro86, pro87, pro89, pro184, pro205, pro206, pro207, pro209, pro210, pro211, pro221, pro241, pro246, pro248 and pro256.

Even more preferably, the first nucleic acid sequences are selected from the group consisting of pro2, pro4, pro8, pro14, pro115, pro116, pro22, pro49, pro74, pro86, pro87, pro89, pro205, pro221, pro246,

Most preferably, the first nucleic acid sequences are selected from the group consisting of pro221, pro210, pro71, pro41, pro30, pro2, pro209, pro14, pro4, pro8, pro246, pro16, pro27, pro5, pro49, pro19, pro140, pro139, pro207, pro81, pro273 and pro362.

In another preferred embodiment the first nucleic acid sequences are selected from the group consisting of pro1, pro2, pro3, pro4, pro5, pro6, pro7, pro8, pro9, pro10, pro12, pro13, pro14, pro15, pro16, pro17, pro18, pro19, pro20, pro21, pro22, pro23, pro24, pro25, pro26, pro27, pro28, pro29, pro30, pro31, pro32, pro34, pro36, pro37, pro38, pro39, pro40, pro41, pro42, pro43, pro44, pro45, pro46, pro47, pro48, pro49, pro50, pro51, pro52, pro53, pro54, pro55, pro56, pro57, pro58, pro59, pro60, pro61, pro62, pro63, pro64, pro65, pro66, pro67, pro68, pro69, pro70, pro71, pro72, pro73, pro74, pro75, pro76, pro77, pro78, pro79, pro80, pro81, pro82, pro83, pro84, pro85, pro86, pro87, pro88, pro89, pro90, pro91, pro92, pro93, pro94, pro95, pro96, pro97, pro98, pro99, pro100, pro101, pro103, pro104, pro105, pro106, pro107, pro108, pro109, pro110, pro111, pro112, pro113, pro114, pro115, pro116, pro117, pro118, pro119, pro120, pro121, pro122, pro123, pro124, pro125, pro126, pro127, pro128, pro129, pro130, pro131, pro133, pro134, pro135, pro136, pro137, pro138, pro139, pro140, pro141, pro142, pro143, pro144, pro145, pro146, pro147, pro148, pro149, pro150, pro152, pro153, pro154, pro155, pro156, pro157, pro158, pro159, pro160, pro161, pro162, pro163, pro164, pro165, pro166, pro167, pro168, pro169, pro171, pro172, pro173, pro174, pro175, pro176, pro177, pro178, pro179, pro180, pro181, pro182, pro183, pro184, pro185, pro187, pro189, pro191, pro193, pro194, pro195, pro196, pro197, pro198, pro199, pro201, pro202, pro203, pro204, pro205, pro206, pro207, pro209, pro210, pro211, pro212, pro213, pro216, pro217, pro219, pro220, pro221, pro222, pro223, pro224, pro225, pro227, pro228, pro229, pro230, pro231, pro233, pro234, pro235, pro236, pro237, pro238, pro239, pro240, pro241, pro242, pro243, pro244, pro245, pro246, pro248, pro249, pro250, pro251, pro253, pro254, pro255, pro256, pro257, pro258, pro259, pro260, pro269, pro278, pro282, pro283, pro284, pro285, pro297, pro315, pro326, pro327, pro328 and pro329.

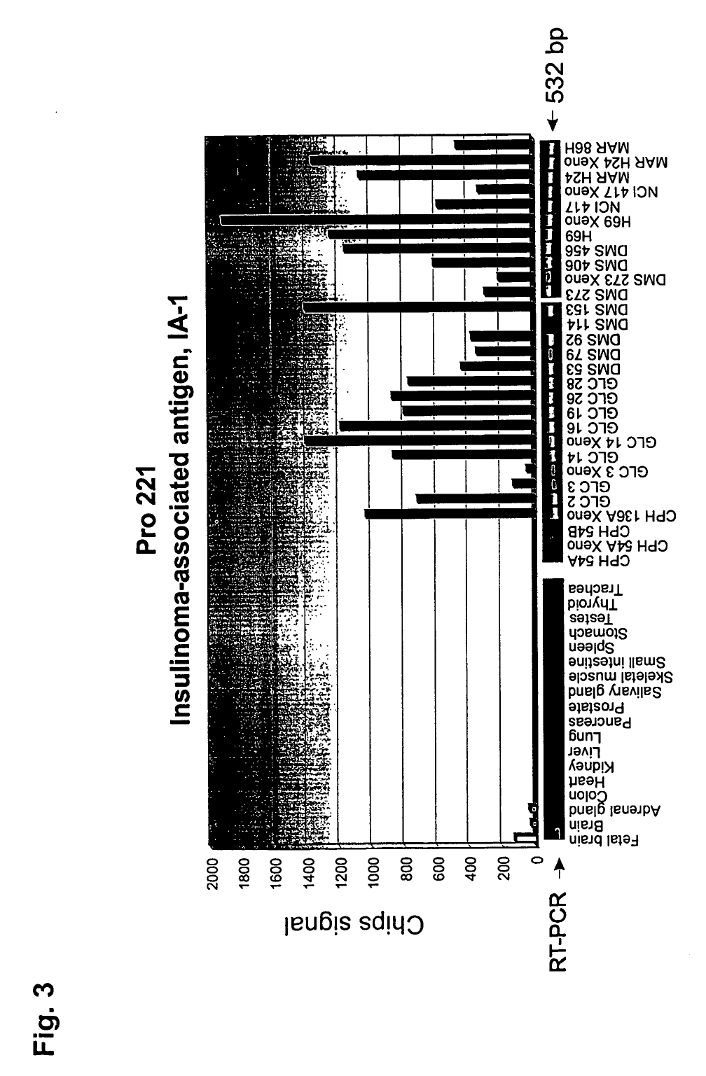

In one especially preferred embodiment of the present invention, the first nucleic acid sequence is pro 221 or a fragment thereof or a functional homologue thereof. Pro221 is the promoter of the gene encoding Insulinoma-associated antigen, IA-1, INSM1. Insulinoma-associated antigen mRNA is expressed at very high levels by all SCLC tested and only expressed at very low levels in brain and adrenal gland. Expression has been demonstrated by for example CHIPS analysis and RT-PCR (see example 1 and FIG. 3).

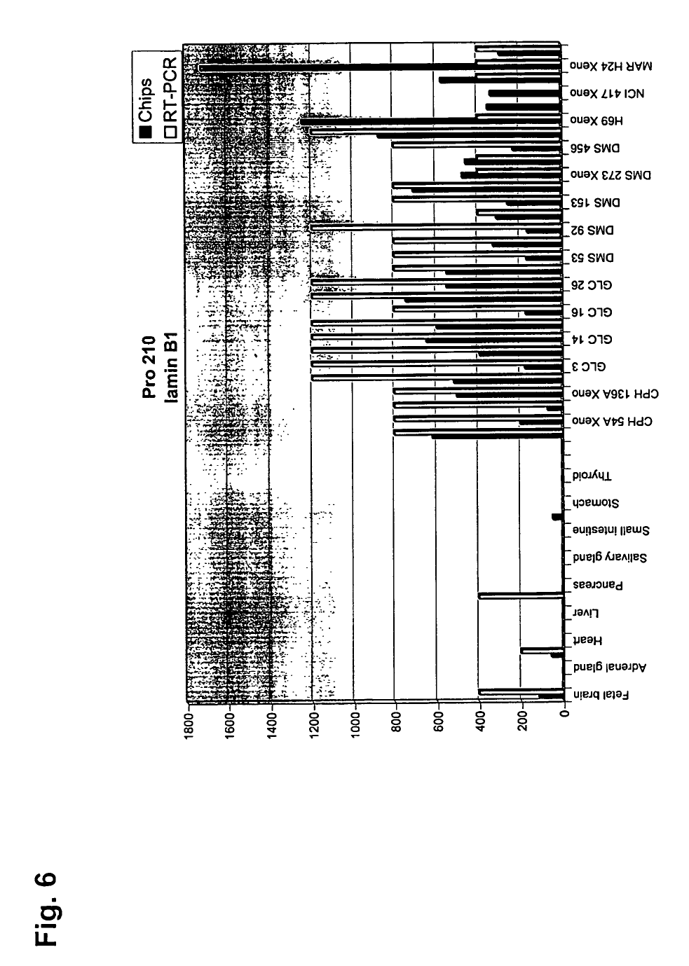

In another preferred embodiment of the present invention the first nucleic acid sequence is pro210 or a fragment thereof or a functional homologue thereof. Pro210 is the promoter of the gene encoding lamin B, LMNB1. LMNB1 mRNA is expressed at very high levels by all tested SCLC and is only expressed at very low levels in spleen, colon and lung. Expression of LMNB1 has been demonstrated by for example CHIPS analysis and RT-PCR (see example 1 and FIG. 6).

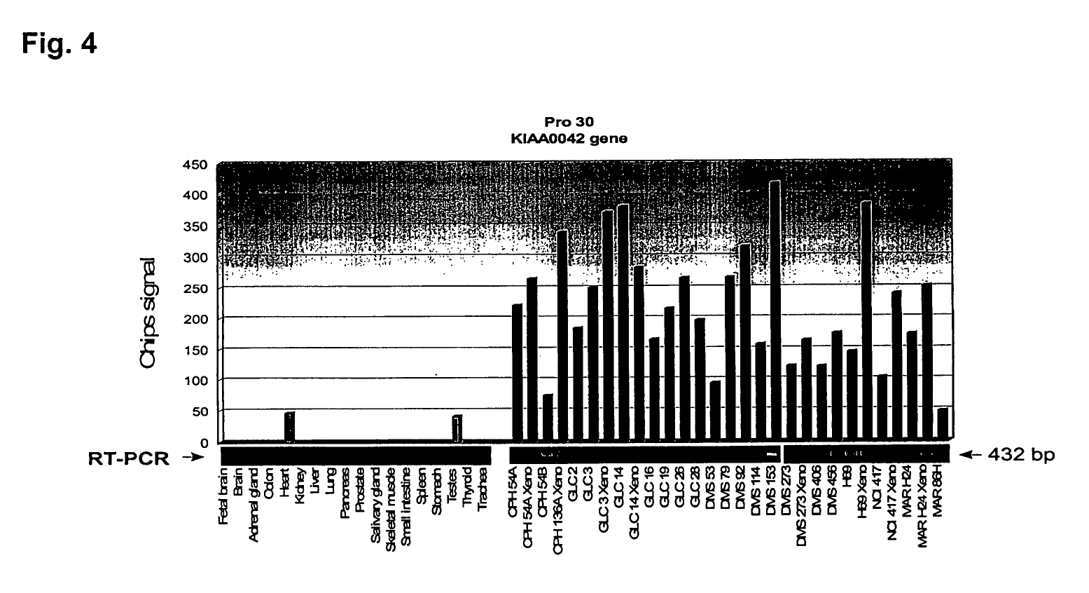

In another preferred embodiment of the present-invention the first nucleic acid sequence is pro30 or a fragment thereof or a functional homologue thereof. Pro30 is the promoter of the human KIAA0042 gene. KIAA0042 RNA is expressed at very high levels in most SCLC and in normal tissues it is only expressed at low levels in testes. Expression of KIAA0042 has been demonstrated by for example CHIPS analysis and RT-PCR (see example 1 and FIG. 4).

In another preferred embodiment of the present invention the first nucleic acid sequence is pro14 or a fragment thereof or a functional homologue thereof. Pro14 is the promoter of the human KIAA0101 gene. KIAA0101 RNA is expressed at high levels in most SCLC and in normal tissues it is expressed at low levels in 7 tissues. Expression of KIAA0101 has been demonstrated by for example CHIPS analysis (see example 1).

In another preferred embodiment of the present invention the first nucleic acid sequence is pro8 or a fragment thereof or a functional homologue thereof. Pro8 is the promoter of the human gene encoding serine/threonine kinase, STK-1, STK12, fms-related tyrosine kinase 3. STK-1mRNA is expressed at high levels in SCLC and in normal tissues it is expressed at low levels in colon, spleen and testes. Expression of STK-1 has been demonstrated by for example CHIPS analysis (see example 1).

In another preferred embodiment of the present invention the first nucleic acid sequence is pro246 or a fragment thereof or a functional homologue thereof. Pro246 is the promoter of the human gene encoding Achaete scute homologous protein, ASH1, ASCL1. ASH1 mRNA is expressed at high levels in many SCLC and in normal tissues it is expressed at low levels in brain. Expression of ASH1 has-been demonstrated by for example. CHIPS analysis (see example 1).

In another preferred embodiment of the present invention the first nucleic acid sequence is pro16 or a fragment thereof or a functional homologue thereof. Pro16 is the promoter of the human gene encoding DNA topoisomerase II alpha (170 kD), TOP2A. TOP2A mRNA is expressed at high levels in SCLC and in normal-tissues it is expressed at low levels in 9 tissues and at high levels in testes. Expression of TOP2A has been demonstrated by for example CHIPS analysis (see example 1).

In another preferred embodiment of the present invention the first nucleic acid sequence is pro27 or a fragment thereof or a functional homologue thereof. Pro27 is the promoter of the human gene encoding Cyclin B2, CCNB2. Cyclin B2 mRNA is expressed at high levels in SCLC and in normal tissues it is expressed at low levels in spleen and trachea and at high levels in testes. Expression of Cyclin B2 has been demonstrated by for example CHIPS analysis (see example 1).

In another preferred embodiment of the present invention the first nucleic acid sequence is pro19 or a fragment thereof or a functional homologue thereof. Pro19 is the promoter of the human gene encoding Kinesin-like spindle protein HKSP, KNSL1. KNSL1mRNA is expressed at high levels in SCLC and in normal tissues it is expressed at low levels in colon, small intestine and testes. Expression of KNSL1 has been demonstrated by for example CHIPS analysis (see example 1).

In another preferred embodiment of the present invention the first nucleic acid sequence is pro139 or a fragment thereof or a functional homologue thereof. Pro139 is the promoter of the human gene encoding Serine/threonine protein kinase SAK. SAK mRNA is expressed at high levels in most SCLC and in normal tissues it is expressed at high levels in testes. Expression of SAK has been demonstrated by for example CHIPS analysis (see example 1).

In another preferred embodiment of the present invention the first nucleic acid sequence is pro207 or a fragment thereof or a functional homologue thereof. Pro207 is the promoter of the human gene encoding Enhancer of zeste homolog 2 (EZH2). EZH2 mRNA is expressed at high levels in SCLC and in normal tissues it is expressed at high levels in testes. Expression of EZH2 has been demonstrated by for example CHIPS analysis (see example 1).

In another preferred embodiment of the present invention the first nucleic acid sequence is pro81 or a fragment thereof or a functional homologue thereof. Pro81 is the promoter of the human HSU79266_gene. HSU79266_RNA is expressed at high levels in most SCLC and in normal tissues it is expressed in testes and spleen. Expression of HSU79266 has_been demonstrated by for example CHIPS analysis (see example 1).

Functional Homologues

Functional homologues of polypeptides according to the present invention is meant to comprise any polypeptide sequence which comprise the same function. For example functional homologues of cell surface molecules are molecules associated with the cell surface which can associate with a binding partner and preferably is capable of internalising the binding partner. Functional homologues of binding partners are molecules which can associate with the cell surface molecule and which preferably is capable of being internalised into cells expressing the cell surface molecule.

Functional homologues according to the present invention comprise polypeptides with an amino acid sequence, which are sharing at least some homology with the predetermined polypeptide sequences as outlined herein above. For example such polypeptides are at least about 40 percent, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with any of the predetermined polypeptide sequences as outlined herein.

The homology between amino acid sequences may be calculated using well known algorithms such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Functional homologues may comprise an amino acid sequence that comprises at least one substitution of one amino acid for any other amino acid. For example such, a substitution may be a conservative amino acid substitution or it may be a non-conservative substitution.

Functional homologues according to the present invention may comprise more than one such substitution, such as e.g. two amino acid substitutions, for example three or four amino acid substitutions, such as five or six amino acid substitutions, for example seven or eight amino acid substitutions, such as from 10 to 15 amino acid substitutions, for example from 15 to 25 amino acid substitution, such as from 25 to 30 amino acid substitutions, for example from 30 to 40 amino acid substitution, such as from 40 to 50 amino acid substitutions, for example from 50 to 75 amino acid substitution, such as from 75 to 100 amino acid substitutions, for example more than 100 amino acid substitutions.

The addition or deletion of an amino acid may be an addition or: deletion of from 0.2 to 5 amino acids, such as from 5 to 10 amino acids, for example from 10 to 20 amino acids, such as from 20 to 50 amino acids. However, additions or deletions of more than 50 amino acids, such as additions from 50 to 200 amino acids, are also comprised within the present invention.

The polypeptides according to the present invention, including any variants and functional homologues thereof, may in one embodiment comprise more than 5 amino acid residues, such as more than 10 amino acid residues, for example more than 20 amino acid residues, such as more than 25 amino acid residues, for example more than 50 amino acid residues, such as more than 75 amino acid residues, for example more than 100 amino acid residues, such as more than 150 amino acid residues, for example more than 200 amino acid residues.

In addition to the polypeptide compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide back-bone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates, including dimers or unrelated chemical moieties. Such functional equivalents are prepared by linkage of functionalities to groups which are found in fragment including at any one or both of the N- and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with Met-Leu-Phe. Derivatives of the acyl groups are selected from the group of alkylmoieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

Homologues of nucleic acid sequences within the scope of the present invention are nucleic acid sequences, which
i) encodes an RNA and/or a protein with similar biological function; or
ii) is capable of exerting a similar biological influence; and which is
a) at least 50% identical, such as at least 60% identical, for example at least 70% identical, such as at least 75% identical, for example at least 80% identical, such as at least 85% identical, for example at least 90% identical, such as at least 95% identical
b) or able to hybridise to the complementary strand of said nucleic acid sequence under stringent conditions.

A similar biological influence within this context may for example be that the nucleic acid sequence is capable influencing transcription of second nucleic acid sequences operably linked thereto in a fashion similar to functional homologous thereof.

Stringent conditions as used herein shall denote stringency as normally applied in connection with Southern blotting and hybridisation as described e.g. by Southern E.

M., 1975, J. Mol. Biol. 98:503-517. For such purposes it is routine practise to include steps of prehybridization and hybridization. Such steps are normally performed using solutions containing 6× SSPE, 5% Denhardt's, 0.5% SDS, 50% formamide, 100 μg/ml denaturated salmon testis DNA (incubation for 18 hrs at 42° C.), followed by washings with 2× SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is Incorporated herein by reference.

Homologous of nucleic acid sequences also encompass nucleic acid sequences which comprise additions and/or deletions. Such additions and/or deletions maybe internal or at the end. Additions and/or deletions may be of 1-5 nucleotides, such as 0.5 to 10 nucleotide, for example 10 to 50 nucleotides, such as 50 to 100 nucleotides, for example at least 100 nucleotides.

Vaccine

In one embodiment of the present invention the cell surface molecules may be used for the preparation of a vaccine. Preferably such a vaccine is capable of raising an immune response against the cell surface molecule. Such an immune response preferably results in specific killing of cells expressing said cell surface molecule. Most preferably, the cells expressing the cell surface molecule are malignant cells, such as the vaccine results in specific killing of malignant cells.

Accordingly, the vaccine is preferably suitable for ameliorating and/or curative and/or prophylactic treatment of a premalignant and/or malignant condition. Hence, the vaccine preferably should be administrated to an individual suffering from a premalignant and/or malignant conditions, preferably cancer. The individual may be any animal, however preferably the individual is a human being.

It is possible to use either the cell surface molecule or fragments thereof or derivatives thereof as well as nucleic acids encoding the cell surface molecule or, fragments thereof or derivatives thereof. Preferred cell surface molecules to use with such a vaccine are cell surface molecules which are expressed, at a higher level in malignant cells in vivo and/or malignant cell lines than in normal tissues. For example such a cell surface molecule may be identified according to the methods outlined herein above. However, other suitable cell surface molecules may also be employed.

Preferably, the cell surface molecule comprises or essentially consists of or for example is a cell surface molecule mentioned in table 2.

More preferably, the cell surface molecule is selected from the group consisting of NCAM1, NPTXR, LRP8, CHRNA5, GRIA2, GRM8, ITGAV, ITGAE, TNFRSF12, L1CAM, GPR49 and TMEFF1.

In one preferred embodiment the vaccine furthermore comprise a non-self antigen covalently linked to said cell surface molecule. Alternatively, when nucleic acid sequences encoding cell surface molecules are used, the vaccine may comprise second nucleic acid sequences encoding a non-self antigen linked to the nucleic acid sequences.

Examples of non-self antigens which may be used with the present invention are invention are T-cell epitopes, preferably polypeptides or peptide.

It is also possible that the vaccine comprises more than one antigen, such as 2, for example 3, such as 4, for example 5, such as more than 5 different antigens. The antigens may be self antigens or non-self antigens.

The vaccine according to the present invention may furthermore comprise an adjuvant and/or a carrier. The carrier or adjuvant could be any carrier or adjuvant known in the art including functional equivalents thereof. Functionally equivalent carriers are capable of presenting the same antigen in essentially the same steric conformation when used under similar conditions. Functionally equivalent adjuvants are capable of providing similar increases in the efficacy of the composition when used under similar conditions.

Preferably, said compositions comprise potent, nontoxic adjuvants that will enhance and/or modulate the immunogenicity of immunogenic determinants including antigenic determinants including haptenic determinants represent one group of preferred adjuvants. In addition, such adjuvants preferably also elicit an earlier, more potent, or more prolonged immune response. Such an adjuvant would also be useful in cases where an antigen supply is limited or is costly to produce.

Adjuvants pertaining to the present invention may be grouped according to their origin, be it mineral, bacterial, plant, synthetic, or host product. The first group under this classification is the mineral adjuvants, such as aluminum compounds. Antigens precipitated with aluminum salts or antigens mixed with or adsorbed to performed aluminum compounds have been used extensively to augment immune responses in animals and humans. Aluminium particles have been demonstrated in regional lymph nodes of rabbits seven days following immunisation, and it may be that another significant function is to direct antigen to T cell containing areas in the nodes themselves. Adjuvant potency has been shown to correlate with intimation of the draining lymph nodes. While many studies have confirmed that antigens administered with aluminium salts lead to increased humoral immunity, cell mediated immunity appears to be only slightly increased, as measured by delayed-type hypersensitivity. Aluminium hydroxide has also been described as activating the complement pathway. This mechanism may play a role in the local inflammatory response as well as immunoglobulin production and B cell memory. Furthermore, aluminium hydroxide can protect the antigen from rapid catabolism. Primarily because of their excellent record of safety, aluminum compounds are presently the only adjuvants used in humans.

Another large group of adjuvants is those of bacterial origin. Adjuvants with bacterial origins can be purified and synthesised (e.g. muramyl dipeptides, lipid A) and host mediators have been cloned (Interleukin 1 and 2). The last decade has brought significant progress in the chemical purification of several adjuvants of active components of bacterial origin: Bordetella pertussis, Mycobacterium tuberculosis, lipopolysaccharide, Freund's Complete Adjuvant (FCA) and Freund's Incomplete Adjuvant (Difco Laboratories, Detroit, Mich.) and Merck Adjuvant 65 (Merck and Company, Inc., Rahway, N.J.). Additionally suitable adjuvants in accordance with the present invention are e.g. Titermax Classical adjuvant (SIGMA-ALDRICH), ISCOMS, Quil A, ALUN, see U.S. Pat. Nos. 58,767 and 5,554,372, Lipid A derivatives, choleratoxin derivatives, HSP derivatives, LPS derivatives, synthetic peptide matrixes, GMDP, and other as well as combined with immunostimulants (U.S. Pat. No. 5,876,735).

B. pertussis is of interest as an adjuvant in the context of the present invention due to its ability to modulate cell-mediated immunity through action on T-lymphocyte populations. For lipopolysaccharide and Freund's Complete Adjuvant, adjuvant active moieties have been identified and synthesised which permit study of structure-function relationships. These are also considered for inclusion in immunogenic compositions according to the present invention.

Lipopolysaccharide and its various derivatives, including lipid A, have been found to be powerful adjuvants in combination with liposomes or other lipid emulsions. It is not yet certain whether derivatives with sufficiently low toxicity for general use in humans can be produced. Freund's Complete Adjuvant is the standard in most ex-% perimental studies.

Mineral oil may be added to vaccine formulation in order to protect the antigen from rapid catabolism.

Many other types of materials can be used as adjuvants in immunogenic compositions according to the present invention. They include plant products such as saponin, animal products such as chitin and numerous synthetic chemicals.

Adjuvants according to the present invention can also been categorised by their proposed mechanisms of action. This type of classification is necessarily somewhat arbitrary because most adjuvants appear to function by more than one mechanism. Adjuvants may act through antigen localisation and delivery, or by direct effects on cells making up the immune system, such as macrophages and lymphocytes. Another mechanism by which adjuvants according to the invention enhance the immune response is by creation of an antigen depot. This appears to contribute to the adjuvant activity of aluminum compounds, oil emulsions, liposomes, and synthetic polymers. The adjuvant activity of lipopolysaccharides and muramyl dipeptides appears to be mainly mediated through activation of the macrophage, whereas B. pertussis affects both macrophages and lymphocytes. Further examples of adjuvants that may be useful when incorporated into immunogenic compositions according to the present invention are described in U.S. Pat. No. 5,554,372.

In one preferred embodiment, adjuvants according to the present invention are selected from the group consisting of aluminium compounds, Freunds incomplete adjuvant, Titermax classical adjuvant and oil emulsions.

There is also provided an embodiment of the present invention wherein the immunogenic composition further comprises a carrier. The carrier may be present independently of an adjuvant. The purpose of conjugation and/or co-immunisation of an antigen and a carrier can be e.g to increase the molecular weight of the antigen in order to increase the activity or immunogenicity of the antigen, to confer stability to the antigen, to increase the biological activity of the determinant, or to increase its serum half-life. The carrier protein may be any conventional carrier including any protein suitable for presenting antigens. Conventional carrier proteins include, but are not limited to, keyhole limpet hemocyanin, serum proteins such as transferrin, bovine serum albumin, or human serum albumin, an ovalbumin, immunoglobulins, or hormones, such as insulin.,

While any suitable pharmaceutical carrier known to those of ordinary skill in the art may be employed in the vaccine of this invention, the type of pharmaceutical carrier will vary depending on the mode of administration and whether a sustained release administration is desired. For parenteral administration, such as subcutaneous injection, the pharmaceutical barrier may e.g. comprise water, saline, alcohol, fat, a wax or a buffer. Biodegradable microspheres (e.g., polylactic galactide) may also be employed as pharmaceutical carriers for the pharmaceutical compositions of this invention.

In one embodiment of the present invention, the vaccine involves the use of dendritic cells. Such an embodiment preferably comprises the steps of
i), providing dendritic cells; and
ii) transferring nucleic acid sequences encoding a cell surface molecule according to the present invention operably linked to second nucleic acid sequences directing expression thereof to the dendritic cells or transferring a cell surface molecule or a fragment thereof to the dendritic cells; and
iii) displaying said cell surface molecules or fragments thereof on the cell surface of the dendritic cells; and
iv) transferring said dendritic cells to the individual to be treated

Preferably, the dendritic cells are cells derived from the individual to be treated, however the dendritic cells may also be derived from another individual. When the dendritic cells are derived from another individual, preferably, the cells are derived from the same species as the individual to be treated. For example, if the individual to be treated is a human being, preferably, the dendritic cells are derived from a human being.

Preferably, the cell surface molecules are displayed on the cell surface as fragments, such as peptide fragments in the context on MHC molecules.

Drug Target

Cell surface molecules, which are capable of binding a binding partner and internalising said binding partner into cells expressing said cell surface molecule, may also be used as drug targets. Preferably, such cell surface molecules are expressed at a different level in malignant cell lines compared with normal tissues. More preferably, the cell surface molecules are identified according to the methods outlined in the present invention.

More preferably the cell surface molecule comprises or essentially consists of or for example is a cell surface molecule mentioned in table 2.

Yet more preferably, the cell surface molecule is selected from the group consisting of NCAM1, NPTXR, LRP8, CHRNA5, GRIA2, GRM8, ITGAV, ITGAE, TNFRSF12, L1CAM, GPR49 and TMEFF1.

A drug target within the scope of the present invention is a molecule, which can be used as a bait, to identify molecules that associates with the drug target and accordingly are potential candidates for drugs. Especially such drugs can be used in the treatment of a premalignant and/or malignant conditions, when formulated accordingly.

The present invention furthermore is concerned with methods for identifying novel drug targets, which can interact with the binding partners according to the present invention (see herein below).

Preferably, such a novel drug target comprise a polypeptide, which is a cell surface molecule expressed at a different level in malignant cells compared with normal cells.

Additionally, the present invention also is concerned with the novel drug targets identified by the above methods.

Methods to Identify Binding Partners

The present invention furthermore provides methods of identifying specific binding partners. Additionally, the invention provides methods of preparing specific binding partners.

A specific binding partner may be identified/prepared by a number of different methods. Any suitable method known to the person skilled in the art may be used with the present invention depending of the specific embodiment.

In one embodiment of the present invention, the binding partner is prepared by standard methods for preparing specific antibodies. For example such a method may involve the following steps:
i) immunising an-animal with said cell surface molecule or a fragment of said cell surface molecule; and
ii) obtaining antibodies from said animal; or
iii) obtaining cells producing antibodies from said animal and obtaining antibodies from said cells

The animal to be immunised may be any animal, preferably a mammal, more preferably the animal is selected from the group comprising rabbit, mouse, rat, donkey, goat and sheep.

The antibodies are preferably obtained from a serum of the immunised animal. They may be purified by any standard method, such as for example by affinity chromatography. Antibodies thus obtained are preferably polyclonal antibodies.

Cell producing antibodies are preferably obtained from the spleen of the immunised animals, preferably the cells are B-cells. The antibody producing cells may be fused with other cells subsequent to purification from the animal, in order to obtain immortal cells. The cells may be cultivated in vitro and antibodies may for example be recovered from the tissue culture supernatant by any standard method such as for example affinity chromatography, or protein A or protein G chromatography. These antibodies are often monoclonal antibodies.

Subsequently, the antibodies may be humanised by any suitable method known to the person skilled in the art.

Antibodies may however also be prepared or identified by other means. For example naturally occurring antibodies may be purified from any suitable animal including a human being. Antibodies may also be obtained from an expression library (see herein below).

In another embodiment of the present invention the binding partner consists of or comprises a polypeptide, which may be identified by screening an expression library. Any suitable expression library may be used with the present invention.

The library may be contained within any suitable host cells, for example the host cells may be bacterial cells, yeast cells, insect cells or mammalian cells. The library may contain nucleic acid sequences encoding polypeptides and/or oligopeptides derived from any species, for example viruses, bacteria, yeast, fungi, plants or animal. Animals may be any animal, preferably mammals, more preferably human beings. The library may also contain nucleic acid sequences encoding polypeptides and/or oligopeptides, which are synthetic and not naturally occurring. The nucleic acid sequences may be contained within any suitable vector, for example a plasmid, a virus, a virus derived vector, a phage, an artificial chromosome or a cosmid.

For example the binding partners may be selected from an expression library expressing polypeptides and/or oligopeptides. They may also be selected from a synthetic combinatorial library expressing polypeptides and/or oligopeptides.

The binding partner may furthermore be identified by screening a phage display library of antibodies. Preferably the phage display library is a library of human antibodies.

In yet another embodiment the binding partners are selected from a library of small chemical compounds. Such a library may comprise any number of different chemical, compounds, which may be produced, by a number of different reactions. Suitable libraries such as for example combinatorial libraries are known to the person skilled in the art.

Once a specific binding partner, which can associate with a cell surface molecule or a fragment of a cell surface molecule has been identified/prepared, such a binding partner should preferably be tested for capability of being internalised. Such tests can be performed in a number of suitable ways depending on the nature of the binding partner.

For example, the binding partner may be incubated with cells expressing the cell surface molecule or fragment thereof, with which the binding partner can associate. Following incubation, the presence and/or absence of the binding partner in the cell interior may be detected. Detection may for example be performed taking advantage of that the binding partner may have been labelled with a directly or indirectly detectable label. Alternatively, the presence of the binding partner may be determined using a first species which can interact specifically with the binding partner. Such a species may be directly or indirectly labelled or it may be detected using a second species, which can interact specifically with the first species and which may be labelled. It is possible to used a third species, which can interact with the second, forth which can interacts with the third and so forth.

Binding Partners

The specific binding partners according to the present invention are capable of interacting with at least one cell surface molecule. However, a specific binding partner may be capable of associating with more than one different cell surface molecules.

In one embodiment of the present invention binding partners within are preferably binding partners, which are capable of being internalised into a cell expressing a cell surface molecule following association with the cell surface molecule.

The binding partners according to the present invention may be identified by any of the methods outlined herein above. However, the binding partner may also be identified by any other method known to the person skilled in the art.

Preferably, the binding partner according to the present invention is capable of associating with one or more cell surface molecules selected from the group consisting of receptors which belong to one of the following groups:
Members of receptor tyrosine kinases
Members of the integrin family
Members of the immunoglobulin superfamily adhesion molecules
Members of the heparan sulfate proteoglycan family
Members of the chondroitin sulfate proteoglycan family
Members of the MAGE family
Members of the RAGE family
Members of the low density lipoprotein receptor family
Members of the cadherin adhesion molecules.
Members of the metabotropic glutamate receptors
Members of the steroid hormone families
Members of the seven transmembrane receptor family
Atrial natriuretic peptide clearance receptor
GFRA3
Transferrin receptor
Members of the serine/threonine kinase receptors

More preferably, the binding partner according to the present invention is capable of associating with one or more cell surface molecules selected from the group consisting of NCAM1, NPTXR, LRP8, CHRNA5, GRIA2, GRM8, ITGAV, ITGAE, TNFRSF12, L1CAM, GPR49 and TMEFF1.

Yet more preferably, the binding partner may associate with one or more fragments of a cell surface molecule. Preferred fragments of cell surface molecules are outlined herein above. Most preferably, the fragments of the cell surface molecules are derived from the extracellular part of the cell surface molecule.

It is preferred that the binding partners according to the present invention may be used in pharmaceutical compositions for the treatment of a premalignant and/or malignant conditions.

In one embodiment of the present invention the binding partner comprises or, essentially consists of a polypeptide or an oligopeptide. A polypeptide and/or an oligopeptide according to the present invention may be naturally occurring or it may be a synthetic polypeptide.

In one preferred embodiment the binding partner is an antibody or a fragment of an antibody. The antibody may be a polyclonal antibody or a binding fragment thereof or it may be a monoclonal antibody or a binding fragment thereof.

The antibody may be derived from an animal, preferably a mammal, more preferably a mammal selected from the group consisting of rat, rabbit, mouse, human, donkey, goat and sheep. In one embodiment the binding partner is a monoclonal antibody derived from a mouse or a rat, for example the binding partner is a murine monoclonal antibody.

The antibody may however also be combinatorial antibody, such as one part of the antibody is derived from one species and the other part is derived from another species. Furthermore, the antibody may be a synthetic antibody, which is not naturally produced.

For many purposes it is preferred that the antibody is a humanised antibody. Especially, if the binding partner should be used for the treatment of a premalignant and/or malignant conditions in a human being, it is desirable that the antibody is humanised.

The antibody may also be human antibody. A human antibody may be a naturally produced human antibody or it may be identified from a phage display library. Furthermore it may be a combinatorial antibody that also comprise parts derived from human antibodies, for example identified from a combinatorial library. Such an antibody need no further humanisation.

The antibody preferably, may interact with the extracellular part of the cell surface molecule (see herein above). The antibody may also associate with a posttranslational modification of the extracellular part of the cell surface molecule. Alternatively, the antibody may interacts with any of the fragments of the cell surface molecule as outlined herein above.

Most preferably the antibody is capable of being internalised upon association with said cell surface molecule. Many antibodies, which associate with a cell surface molecule, are not internalised into a cell expressing the cell surface molecule upon association. Preferred antibodies within the scope of the present invention are antibodies, which may be internalised into a cell expressing the cell surface molecule following association.

In another embodiment of the present invention the binding partner is a naturally occurring ligand for said cell surface molecule. A naturally occurring ligand is a compound, which under natural conditions associates with the cell surface molecule. A naturally occurring ligand may for example selected from the group consisting of polypeptides, oligopeptides, hormones, lipids, saccharides, amino acids, neurotransmitters, nucleotide, nucleoside and combinations thereof.

A naturally occurring ligand may be purified from an animal including a human being by any conventional technique suitable for the ligand of the embodiment. However, a natural ligand may also be produced in vitro by any method known to the person skilled in the art.

In one embodiment of the present invention the binding partner is a recombinantly produced ligand for said cell surface molecule. If the ligand is a polypeptide or an oligopeptide, the ligand may be produced by transforming a suitable host, such as bacteria, yeast, protozoa, animals such as for example mammals, plants, animal cells or plant cells with a nucleic acid sequence encoding the ligand operably linked to nucleic acid sequences that direct transcriptions and/or translation of the nucleic acid sequence in the particular host. Transformation may be performed according to any conventional technique. Subsequently the ligand may be purified according to any standard method.

In another embodiment the binding partner is a viral protein or comprise a viral protein or fragments thereof. A large number of viral proteins are capable of associating with cell surface molecule. Frequently such an association results in internalisation of the virus particle, and hence viral proteins are suitable binding partners according to the present invention.

Preferably, a viral protein is a viral capsid protein, more preferably the viral capsid protein is capable of being internalised into a cell expressing the cell surface molecule.

The viral protein may be derived from any virus, preferably a virus which is capable of infecting cells which naturally expresses the cell surface molecule. The virus could for example be selected from the group consisting of adenovirus, herpes simplex virus, influenza virus and members of the lentivirus family.

The binding partner may be recombinantly produced (see herein above) and comprise viral capsid protein sequences. Preferably the viral capsid protein sequences are the sequences of the viral protein which can associate with the cell surface molecule and result in internalisation.

In yet another embodiment of the present invention the binding partner is a small chemical compound. Such a small chemical compound is usually synthetically produced. It can be produced by any process or combination of processes known to the person skilled in the art.

Preferred small chemical compounds can interact with the cell surface molecules and/or the fragments of cell surface molecules as outlined herein above. More preferably, the small chemical compounds are capable of being internalised into cells expressing said cell surface molecules.

In one embodiment the binding partner may be a polypeptide selected from the group consisting of EGF, TGFα, TGF-β, amphiregulin, HB-EGF, epiregulin, beta-cellulin, IGF-1, IGF-2, collagen, fibronectin, vitronectin, laminin, amyloid beta-protein precursor, interferon γ, transferrin, autocrine motility factor, L1, NCAM, cadherin, bombesin, neuromedin B, TNF, erythropoietin, interleukin and cholecystokinin B. Furthermore, the binding partner may for example be an organic compound selected from the group consisting of cannabinoid, acetylcholin, dopamine, norepihrine, serotonin and GABA. In addition the binding partner may for example be an oligopeptide selected from the group consisting of formyl peptide and atrial natriuretic peptide. Furthermore the binding partner may for example be an amino acid, the binding partner may be any amino acid, preferably the amino acid is selected from the group consisting of glutamate, glycine and histamine. Additionally, the binding partner may for example be a nucleotide selected from the group consisting of ATP and GTP. Furthermore, the binding partner may be a hormone such as estrogen, a lipid or a saccharide.

Furthermore, the binding partner according to the present invention may be selected from the group consisting of EGF, TGF-α, heregulins, Insulin, IGF-1, PDGF, CSF-1, SCF, Flt-3L, VEGF, FGFs1-9, NGF, BDNF, NT-3, NT-4, HGF, MSP, Gas6, Angiopoietin-1, ephrinA1-5, ephrinB1-3, GDNF, PEPHC1, TGF-β, Angiotensin, Thrombin, Adenosine, Adrenalin, Serotonin, deltorphin, Dopamine, PTH, Secretin, VIP, PACAP, Glucagon, CRF, Bombesin, Bradykinin, NPY, Glutamate, Ca²⁺, GABA, Chemokines and Opioids.

More preferably, the binding partner may be selected from the group consisting of L-glutamate, kainate, 5-(bromomethyl)-4-isoxazolepropionic acid ( ), analogues of glutamate, substituted quinoxaline 2,3 diones, GYK152466, 5-1-Willardine, 5-FWillardine, agonist and antagonist ligands to the AMPA ((RS)-α-Amino-3-hydroxy-5-methylisoxazolepropionic acid, NBQX, CNQX, DNQX, GYKI 52466, 6-Chlorokynurenic acid, JSTX, L-APA, L-SOP, ACPT, (R,S)-PPG, CPPG, MAP4, (S)-3,4-DCPG, vitronectin, cytactin, fibronectin, fibrinogen, laminin, MMP-2, osteopontin, prothrombin, thrombospondin, von Willebrandts Factor, recombinant fragments of L1CAM, salmosin, E-cadherin and peptides thereof, including the peptide: NRDKETKIV, NCAM1 domain Ig I+II, NCAM1 domain IgIII and peptides thereof, peptides C3: ASKKPKRNIKA (SEQ ID NO. 305), C3: AKKERQRKDTU (SEQ ID NO. 306), D4: ARALNWGAKP (SEQ ID NO 307), monoclonal antibody 123C3, NPTX1, NPTX2, taipoxin, TCBP49, Oxynor, ApoE2, ApoE3, ApoE4, peptides from ApoE (E_{141;-155}; LRKLRKRLLRDADDL (SEQ ID NO 308) and its tandem E_{(141;-155)2}; LRKLRKRLLRDADDL-LRKLRKRLL RDADDL (tandem of SEQ ID NO:308)) reelin, nicotine, acetylcholine, α-bungarotoxin and carbachol.

The binding partner according to the present invention should be selected according to cell surface molecule employed in the specific embodiment of the invention.

Hence, in embodiments of the invention, wherein the cell surface molecule is capable of internalising a binding partner or a targeting complex, the binding partner is preferably selected from the group consisting of NCAM1 domain Ig I+II, NCAM1 domain IgIII and peptides thereof, peptides C3: ASKKPKRNIKA (SEQ ID NO 305), D3: AKKERQRKDTU SEQ ID NO 306), D4: ARALNWGAKP (SEQ ID NO 307), monoclonal antibody 123C3, NPTX1, NPTX2, taipoxin, TCBP49, Oxynor, ApoE2, ApoE3, ApoE4, peptides from ApoE (E_{141;-155}; LRKLRKRLLRDADDL (SEQ ID NO 308) and its tandem E_{(141;-155)2}; LRKLRKRLLRDADDL-LRKLRKRLL RDADDL (tandem of SEQ ID NO 308)) reelin, nicotine, acetylcholine, α-bungarotoxin, carbachol and specific internalising antibodies directed against said cell surface molecules.

In one especially preferred embodiment of the present invention the cell surface molecule is NCAM1. When the cell surface molecule is NCAM1, then the binding partner is preferably selected from the group consisting of the first and second Immunoglobulin domains (Ig) of NCAM1 (Kiselyov et al., 1997), the third Ig domain of NCAM1, the adhesion molecule L1 and proteoglycans. Furthermore, the binding partner may preferably be selected from the group consisting of synthetic binding partners capable of associating with NCAMs including for example a large number of peptides (11 amino acids) identified from a combinatorial peptide library (Ronn et al., 1999), including for example C3: ASKKPKRNIKA (SES ID NO 305), D3: AKKERQRKDTU (SEQ ID NO 306) and D4: ARALNWGAKPK (SEQ ID NO 307)(Rønn et al., 1999). In addition the binding partner may preferably be selected from the group consisting of antibodies against NCAM1, preferably monoclonal antibodies against NCAM1, for example antibody (123C3) which causes internalisation.

In another preferred embodiment of the invention the cell surface molecule is NPTXR. When the cell surface molecule is NPTXR, then the binding is preferably selected from the group consisting of Neuronal pentraxin 1 (NP1, NPTXI) and Neuronal pentraxin 2 (NP2, NPTX2) (Kirkpatrick et al., 2000; Dodds et al., 1997). Furthermore, the binding partner may preferably be selected from the group consisting of the snake venom taipoxin and taipoxin associated calcium-binding protein 49 (TCBP49) and the taipoxin analogue, Oxynor. In addition the binding partner may preferably be selected from the group consisting of antibodies against NPTXR, preferably monoclonal antibodies against NPTXR.

In another preferred embodiment of the invention the cell surface molecule is LRP8.

When the cell surface molecule is LRP8, then the binding partner is preferably selected from the group consisting of ApoE2, ApoE3 and ApoE4 and reelin. Furthermore, the binding partner may preferably be selected from the group consisting variouys recombinant ApoE isoforms some of which are commercially available. However, the natural ApoE isoforms are capable of associating with several receptors. In addition, the binding partner may preferably be selected from the group consisting peptides from ApoE, for example (E_{141;-155}; LRKLRKRLLRDADDL (SEQ ID NO 308) and its tandem E_{(141;-155)2}; LRKLRKRLLRDADDL-LRKLRKRLL RDADDL), both have been shown to inhibit receptor function (Riddell et al., 1999). In addition the binding partner may preferably be selected from the group consisting of antibodies against LRP8, preferably monoclonal antibodies against LRP8.

In another preferred embodiment of the invention the cell surface molecule is CHRNA5. When the cell surface molecule is CHRNA5, then the binding partner is preferably selected from the group consisting of nicotine, acetylcholine and the toxin α-bungarotoxin. Furthermore, the binding partner may be selected from the group consisting (of synthetic agonists of CHRNA5, for example carbachol. In addition the binding partner may preferably be selected from the group consisting of antibodies against CHRNA5, preferably monoclonal antibodies against CHRNA5.

In another preferred embodiment of the invention the cell surface molecule is L1CAM. When the cell surface molecule is L1CAM, then the binding partner may for example comprisean adhesion molecule of the integrin family or a fragment thereof. L1CAM is known to bind several adhesion molecules of the integrin family through an RGD sequence and of the immunoglobulin family via an oligomannosidic carbohydrate. In addition the binding partner may preferably be selected from the group consisting of antibodies against L1CAM, preferably monoclonal antibodies against L1CAM.

In another preferred embodiment of the invention the cell surface molecule is TNFRSF12. When the cell surface molecule is TNFRSF12, then the binding partner may for example an antibody against TNFRSF12, preferably a monoclonal antibody against TNFRSF12, for example a monoclonal antibody to the extracellular domain of TNFRSF12.

In one especially preferred embodiment of the present invention the cell surface molecule is GRIA2. When the cell surface molecule is GRIA2, then the binding partner is preferably selected from the group consisting of L-glutamate and kainate. Furthermore, the binding partner may preferably be selected from the group consisting of synthetic ligands to GRIA2, for example agonist and antagonist ligands to the AMPA ((RS)-α-Amino-3-hydroxy-5-methyl-4-isoxazolepropionic acid) receptors. AMPA receptor ligands are generally either analogues of glutamate or substituted quinoxaline 2,3 diones. The antagonists are divided into competitive and modulatory site antagonists (reviewed in (Bräuner-Osborne et al, 2000; Madsen et al., 2001)). In addition, one AMPA antagonist, GYKI52466, has been shown to inhibit tumor cell growth (Cavalheiro and Olney, 2001) on cells expressing the GRIA2 receptor. As the receptor binding of the ligands involves binding of the mayor portions of the ligand, substitutions (such as halogens) can only be performed at very few sites. A brome substituted form of AMPA (ABPA) was shown to act as a potent agonist of AMPA receptors (Krogsgaard-Larsen et al., 1985). The agonist may also be a halogenated form of an agonist, for example williardiine and analogues with different AMPA receptor affinities (Jane. D, 2001). Many of these show many fold higher affinity for AMPA receptors than AMPA itself. Synthesis of Williardiine and 6-azowillardiine halogenated analogues is described in detail in (Jane et al., 1997). 5-I-Willardine and 5-F-Willardine are commercially available, also in a ³H-forms. In addition the binding partner may selected from the group consisting of small molecule antagonists, for example the commercially available NBQX, CNQX, DNQX, GYKI 52466 and 6-Chlorokynurenic acid and the group of larger polyamine antagonists of AMPA receptor channels related to the spider toxin JSTX-3 (Yoneda et al., 2001). In addition the binding partner may preferably be selected from the group consisting of antibodies against GRIA2, preferably monoclonal antibodies against GRIA2.

In another preferred embodiment of the invention the cell surface molecule is GRM8. When the cell surface molecule is GRM8, then the binding partner may preferably be L-glutamate. Furthermore, the binding partner may preferably be selected from the group consisting of agonists and antagonists, for example the commercially available L-APA, L-SOP, ACPT, (R,S)-PPG, CPPG, MAP4, (S)-3,4-DCPG and MSOP and their ³H labelled forms. One agonist, (R,S)-PPG has a 25 fold preference for GRM8 (Gasparini et al, 1999) and the agonist (S)-3,4-DCPG displays more than 100 fold selectivity for GRM8 (Bruno et al., 2001; Thomas et al., 2001; Turner and Salt, 1999). In addition the binding partner may preferably be selected from the group consisting of antibodies against GRM8, preferably monoclonal antibodies against GRM8.

In another preferred embodiment of the invention the cell surface molecule is ITGAV. When the cell surface molecule is ITGAV, then the binding partner may preferably be selected from the group consisting of vitronectin, cytactin, fibronectin, fibrinogen, laminin, MMP-2, osteopontin, prothrombin, thrombospondin von Willebrandts Factor and αvβ3. αvβ3 has been shown to bind recombinant fragments of the neural cell adhesion molecule L1 though the αv subunit (Montgomery et al., 1996). The natural ligands, such as vitronectin, are also ligands for a number of other, ubiquitously expressed integrins and therefore not optimal for specific targeting. Furthermore, the binding partner may preferably be selected from the group consisting of disintegrins and ADAMs, for example salmosin or contortrostatin. Disintegrins and ADAMs (A Disintegrin and A Metalloprotease) are a large number of proteins from snake venoms, which bind with different specificities to different integrins (Evans, 2001; Huang, 1998). Several disintegrins specific for αvβ3 and αvβ5 have been identified, including recombinantly produced salmosin (Kang et al., 1999) and contortrostatin (Mercer et al., 1998). In addition, the binding partner may preferbly be selected from the group consisting of small cyclic peptides and non-peptide compounds, which are antagonists of αvβ3 binding (Boger et al., 2001; Hartman and Duggan, 2000; Kerr et al., 2000; Batt et al., 2000). In addition the binding partner may preferably be selected from the group consisting of antibodies against ITGAV, preferably monoclonal antibodies against ITGAV.

In another preferred embodiment of the invention the cell surface molecule is ITGAE. When the cell surface molecule is ITGAE, then the binding partner may preferably be the cell adhesion molecule E-cadherin or a fragment thereof. The heterophilic binding site on E-cadherin for αEβ7 differs from the homophilic binding site of E-cadherin with another E-cadherin (Karecla et al., 1996; Taraszka et al, 2000). Preferably the fragment comprises or even more preferably consists of a short peptide sequence from the first domain of E-cadherin (amino acids 27-34: NRDKETKV (SEQ ID NO 309), which are capable of interfering with the binding of αEβ7 to E-cadherin. Furthermore, the binding partner may be selected from the group consisting of specific αEβ7 specific peptides (Brenner and Cepek, 2001). In addition the binding partner may preferably be selected from the group consisting of antibodies against ITGAE, preferably monoclonal antibodies against ITGAE, such as αE specific antibodies that may be used as antagonists.

Complex

In one embodiment the present invention relates to a complex comprising a cell surface molecule and a binding partner. Preferably the cell surface molecule is identified by the method disclosed by the present invention. Preferably, the cell surface molecule comprises or essentially consists of or for example is a cell surface molecule mentioned in table 2.

More preferably the cell surface molecule may be selected from the group consisting of
Members of receptor tyrosine kinases
Members of the integrin family
Members of the immunoglobulin superfamily adhesion molecules
Members of the heparan sulfate proteoglycan family
Members of the chondroitin sulfate proteoglycan family
Members of the MAGE family
Members of the RAGE family
Members of the low density lipoprotein receptor family
Members of the cadherin adhesion molecules
Members of the metabotropic glutamate receptors
Members of the steroid hormone families
Members of the seven transmembrane receptor family
Atrial natriuretic peptide clearance receptor
GFRA3
Transferrin receptor
Members of the serine/threonine kinase receptors

More preferably, the cell surface molecule is selected from the group consisting of NCAM1, NPTXR, LRP8, CHRNA5, GRIA2, GRM8, ITGAV, ITGAE, TNFRSF12, Li CAM, GPR49 and TMEFF1.

The binding partner of the complex may be any specific binding partner capable of interacting with the cell surface molecule. Examples of binding partners are given herein above.

Targeting Complex

The present invention provides targeting complexes, which comprise a binding partner and a bioreactive species. The binding partner should be capable of associating with one or more cell surface molecules or fragments thereof as outlined herein above.

In one preferred embodiment of the present invention, the cell surface molecule, which can associate with the binding partner of the targeting complex, is capable of internalising the targeting complex. However, in another preferred embodiment of the present invention, the cell surface molecule is not capable of internalising the targeting complex, but merely is capable of associating with the targeting; complex.

More preferably, the cell surface molecule comprises or essentially consists of or for example is Transferrin receptor; such as type II membrane protein clone: for example is HP10481; such as type II membrane protein clone: such as HP10390; for example is PG40; such as TRC8; for example is TR2-11; such as OA3 antigenic surface determinant; for example is integrin alpha 6, For example GPIIb; such as vitronectin receptor alpha subunit; for example is integrin alpha-7; such as integrin alpha E precursor; for example is integrin alpha 6B; such as integrin alpha 5 subunit; for example is integrin beta-5 subunit; such as integrin alpha-3 chain; for example is RYK; such as amyloid precursor protein-binding protein 1; for example is putative transmembrane GTPase; such as membrane cofactor protein; FOR EXAMPLE GLVR1; for example is Mr 110,000 antigen; for example is syndecan-1; such as putative seven transmembrane domain protein; for example is LCA-homolog/LAR protein; such as M6 antigen; for example is Me491CD63 antigen; such as multispanning membrane protein; for example is DDR; such as autocrine motility factor, receptor; for example is insulin receptor precursor; such as IGF1R, for example is insulin-like growth factor II receptor; such as SAS; for example is TAPA-1; such as MICB; for example is MHC class II HLA-DR7-associated glycoprotein beta-chain; such as HLA-DP; for example is bone small proteoglycan I biglycan; such as CAR; for example is MEA11; such as interferon-gamma receptor alpha chain; for example is Polymeric immunoglobulin receptor; such as metabotropic glutamate receptor type 4; for example is metabotropic glutamate receptor 8; such as CLPTM1; for example is MAGE4b; such as MAGE5a; for example is MAGE-3; such as MAGE-1; for example is MAGE6; such as MAGE-9; for example is MAGE11; such as CD24; for example is CD59; such as CD44; for example is low density lipoprotein receptor; such as very low density lipoprotein receptor; for example is N-CAM; such as lamin B receptor homolog TM7SF2; for example is putative T11ST2 receptor binding protein precursor; such as NTR2 receptor; for example is RAGE-4; such as HLA-G1; for example is MOAT-C; such as alpha 2 delta calcium channel subunit isoform I; for example is LFA-3; such as L1-CAM; for example is AVPR2; such as C1 p115 C1; for example is TE2; such as RbP; for example is HCF1; such as IRAK; for example is CD151; such as surface antigen; for example is MAG; such as GPR19; for example is pcta-1; such as PRAME; for example is vasopressin activated calcium mobilizing receptor-like protein; such as serotonin receptor 5-HT4B; for example is serotonin 1 D receptor (5HT1D˜), such as CD9; for example is LDL receptor member LR3; such as DR6; for example is tumor necrosis factor receptor; such as HG38; for example is urokinase-type plasminogen receptor; such as FGF receptor; for example is nerve growth factor receptor; such as cystine/glutamate transporter; for example is CB1 cannabinoid receptor (CNR1); such as PSG; for example is PSG13′; such as CPE-receptor; for example is CRH2R; such as OCI5; for example is TRAIL receptor 2; such as HNMP-1; for example is kidney alpha-2-adrenergic receptor; such as erythropoietin receptor; for example is chondroitin sulphate proteoglycanversican V1; for example is mGluR1 beta; such as CD97; for example is L6; such as 10. NY-ESO-1; for example is T-cell receptor alpha delta; such as ror1; for example is ror2; such as SSTR2; for example is VESPR; such as IgG Fc receptor; for example is glutamate receptor subunit GluRC; such as HEK2; for example is PVR; such as CEA; for example is CC-chemokine-binding receptor JAB61; such as HER2; for example is HER3; such as hypothetical protein FLJ22357 similar to Epidermal growth 15, factor receptor-related protein; for example is putative endothelin receptor type B-like protein; such as GLVR2; for example is P2X4 purinoreceptor; such as FPRL1; for example is Atrial natriuretic peptide clearance receptor; for example is gastrin/CCK-B receptor; such as Neuromedin B receptor; for example is GFRA3; such as GRPR; for example is CDH1; such as CDH2; for example is TGFBR1, such as 20 TGFBR2; for example is TGFBR3; such as precursor of epidermal growth factor receptor, yet more preferably the cell surface molecule is selected from the group consisting of receptors which belong to one of the following groups:
Members of receptor tyrosine kinases
Members of the integrin family
Members of the immunoglobulin superfamily adhesion molecules.
Members of the heparan sulfate proteoglycan family
Members of the chondroitin sulfate proteoglycan family
Members of the MAGE family
Members of the RAGE family
Members of the low density lipoprotein receptor family
Members of the cadherin adhesion molecules
Members of the metabotropic glutamate receptors
Members of the steroid hormone families
Members of the seven transmembrane receptor family
Atrial natriuretic peptide clearance receptor
GFRA3
Transferrin receptor
Members of the serine/threonine kinase receptors

Even more preferably, the cell surface molecules selected from the group consisting of NCAM1, NPTXR, LRP8, CHRNA5, GRIA2, GRM8, ITGAV, ITGAE, TNFRSF12, L1CAM, GPR49 and TMEFF1.

The bioreactive species according to the present invention may be any speciesi which can directly or indirectly exert a biological influence on a target cell, wherein the target cell, is any cell expressing the cell surface molecule and which can internalise the targeting construct. The biological influence according to the present invention may for example be selected from the group consisting of cell cycle arrest, protection of cell against toxins and cell death.

The bioreactive species may any compound for example it may be a nucleic acid sequence, a polypeptide, an oligopeptide, a toxin, a small chemical compound or a radioactive isotope.

In one preferred embodiment the bioreactive species is a nucleic acid sequence. Preferably, the nucleic acid sequence comprises a second nucleic acid operably linked to a first nucleic acid sequence comprising an expression signal.

The second nucleic acid sequence may in one preferred embodiment encode a therapeutic protein (see herein below). The nucleic acid sequence encoding a therapeutic protein may comprise complementary DNA (cDNA). The term “cDNA” used here, is intended to refer to DNA prepared using messenger RNA (mRNA) as template. The advantage of using a cDNA, as opposed to genomic DNA or DNA polymerised from a genomic DNA or non- or partially-processed RNA template, is that the cDNA does not contain any non-coding intron sequences but, rather comprise the uninterrupted coding region of the corresponding protein. There may be times when the full or partial genomic sequence is preferred, however, such as where the non-coding regions are required for optimal expression.

In another embodiment the second nucleic acid sequence encodes an antisense.

RNA or part of an antisense RNA. Alternatively, the second nucleic acid sequence may comprise or essentially consist of an antisense RNA or part of an antisense RNA.

In the context of the present invention the term “antisense RNA” is intended to encompass an RNA sequence transcribed from the non-coding DNA strand of a gene or an RNA sequence that is capable of hybridising to an mRNA or fragments thereof under stringent conditions.

Preferably, the antisense RNA within the context of the present invention is the antisense RNA of a gene encoding a protein, which promotes cell survival, cell growth and/or cell mobility. More preferably, the antisense RNA is the antisense RNA of an oncogene or a growth factor.

In another embodiment the second nucleic acid sequence encodes or comprises a ribozyme. A ribozyme within the present context is a molecule, which comprises at least one RNA, which comprises an enzymatic activity. Preferably, ribozymes according to the present invention is targeted against RNA of an oncogene or a protooncogene or growth factors.

Accordingly, in preferred embodiments of the present invention antisense RNAs or ribozymes are targeted against RNA of an oncogene or proto-oncogene or growth factors. Examples of growth factors are indicated herein below.

Oncogenes are a diverse class of genes, whose products may contribute to the development and/or advancement of cancer. Proto-oncogenes may under certain circumstances or after due to mutations contribute to the development and/or advancement of cancer. Oncogene or proto-oncogene may for example be selected from the group consisting of Ras, Raf, Myc, Syn, Pim, BMI-1, FOP, S is, KGF, Fms, Flg, Neu, Trk, Kit, Met, Src, Fyn, Mas, Fes/Fps, Tre, Mer, ABL, BCL3, int-2, Cym, Ets, Elk, RhoA, Ski, Wnt-5a, Spi-1, Rap2, p55 and c-tyr. This is not an exhaustive list of oncogenes and proto-oncogenes, which may be used with the present invention, but merely comprises illustrative examples.

The second nucleic acid sequences may also encode a tumour suppressor gene to be introduced into the cell expressing the cell surface molecule in order to correct, any endogenous mutations of said tumour suppressor within the cell. The tumour., suppressor may be any tumour suppressor for example any of the tumour suppressors indicated herein below.

The first nucleic acid sequences according to the present invention preferably comprise an expression signal. Such an expression signal should preferably influence the transcription of second nucleic acid sequences operably linked thereto, Preferably, the first nucleic acids sequences according to the present invention influence transcription such as they enhance transcription under specific circumstances.

In one embodiment of the present invention the first nucleic acid sequence comprises an expression signal, which directs a lower level of expression of a second nucleic acid sequence in malignant cells, compared with non-malignant cells. In another embodiment the first nucleic acid sequence comprises an expression signal, which directs approximately the same level of expression of a second nucleic acid sequence in malignant cells, compared with non-malignant cells.

However, in a preferred embodiment of the present invention the first nucleic acid sequences directs a higher level of expression of a second nucleic acid sequence in malignant cells compared with non-malignant cells. In particular, the first nucleic acid sequences may be selected from the group consisting of first nucleic acid sequences identified according to the methods outlined herein above.

Preferably, the first nucleic acid sequence is selected from the group consisting of pro1, pro2, pro3, pro4, pro5, pro6, pro7, pro8, pro9, pro10, pro12, pro13, pro14, pro15, pro16, pro17, pro18, pro19, pro20, pro21, pro22, pro23, pro24, pro25, pro26, pro27, pro28, pro29, pro30, pro31, pro32, pro34, pro36, pro37, pro38, pro39, pro40, pro41, pro42, pro43, pro44, pro45, pro46, pro47, pro48, pro49, pro50, pro51, pro52, pro53, pro54, pro55, pro56, pro57, pro58, pro59, pro60, pro61, pro62, pro63, pro64, pro65, pro66, pro67, pro68, pro69, pro70, pro71, pro72, pro73, pro74, pro75, pro676, pro77, pro78, pro79, pro80, pro81, pro82, pro83, pro84, pro85, pro86, pro87, pro88, pro89, pro90, pro91, pro92, pro93, pro94, pro95, pro96, pro97, pro98, pro99, pro100, pro101, pro103, pro104, pro105, pro106, pro107, pro108, pro109, pro 110, pro111, pro112, pro113, pro114, pro115, pro116, pro117, pro118; pro119, pro120, pro121, pro122, pro123, pro124, pro125, pro126, pro127, pro128, pro129, pro130, pro131, pro133, pro134, pro135, pro136, pro137, pro138, pro139, pro140, pro141, pro142, pro143, pro144, pro145, pro146, pro147, pro148, pro149, pro150, pro152, pro153, pro154, pro155, pro156, pro157, pro158, pro159, pro160, pro161, pro162, pro163, pro164, pro165, pro166, pro167, pro168, pro169, pro171, pro172, pro173, pro174, pro175, pro176, pro177, pro178, pro179, pro180, pro181, pro182, pro183, pro184, pro185, pro187, pro189, pro191, pro193, pro194, pro195, pro196, pro197, pro198, pro199, pro201, pro202, pro203, pro204, pro205, pro206, pro207, pro208, pro209, pro210, pro211, pro212, pro213, pro215, pro216, pro217, pro219, pro220, pro221., pro222, pro223, pro224, pro225, pro226, pro227, pro228, pro229, pro230, pro231, pro232, pro233, pro234, pro235, pro236, pro237, pro238, pro239, pro240, pro241, pro242, pro243, pro244, pro245, pro246, pro247, pro248, pro249, pro250, pro251, pro253, pro254, pro255, pro256, pro257, pro258, pro259, pro260, pro262, pro263, pro264, pro267, pro268, pro269, pro270, pro271, pro272, pro273, pro275, pro277, pro278, pro279, pro280, pro282, pro283, pro284, pro285, pro286, pro287, pro289, pro290, pro291, pro292, pro293, pro294, pro295, pro296, pro297, pro298, pro299, pro300, pro301, pro302, pro303, pro304, pro305, pro306, pro307, pro308, pro309; pro310, pro311, pro312, pro313, pro315, pro316, pro317, pro318, pro319, pro320, pro321, pro322, pro323, pro324, pro326, pro327, pro328; pro329, pro330, pro331, pro332, pro333, pro334, pro335, pro336, pro337, pro338, pro339, pro340, pro341, pro344, pro346, pro347, pro348, pro349, pro352, pro353, pro354, pro355, pro356, pro358, pro359 and pro361.

The first nucleic acid sequences may furthermore comprise and/or essentially consist of fragments of nucleic acid sequences selected from the group consisting of pro1, pro2, pro3, pro4, pro5, pro6, pro7, pro8, pro9, pro10, pro12, pro13, pro14, pro15, pro16, pro17, pro18, pro19, pro20, pro21, pro22, pro23, pro24, pro25, pro26, pro27, pro28, pro29, pro30, pro31, pro32, pro34, pro36, pro37, pro38, pro39, pro40, pro41, pro42, pro43, pro44, pro45, pro46, pro47, pro48, pro49, pro50, pro51, pro52, pro53, pro54, pro55, pro56, pro57, pro58, pro59, pro60, pro61, pro62, pro63, pro64, pro65, pro66, pro67, pro68, pro69, pro70, pro71, pro72, pro73, pro74, pro75, pro76, pro77, pro78, pro79, pro80, pro81, pro82, pro83, pro84, pro85, pro86, pro87, pro88, pro89, pro90, pro91, pro92, pro93, pro94, pro95, pro96, pro97, pro98, pro99, pro100, pro101, pro103, pro104, pro105, pro106, pro107, pro108, pro109, pro110, pro111, pro112, pro113, pro114, pro115, pro116, pro17, pro118, pro119, pro120, pro121, pro122, pro123, pro124, pro125, pro126, pro127, pro128, pro129, pro130, pro131, pro133, pro134, pro135, pro136, pro137, pro138, pro139, pro140, pro141, pro142, pro143, pro144, pro145, pro146, pro147, pro148, pro149, pro150, pro152, pro153, pro154, pro155, pro156, pro157, pro158, pro159, pro160, pro161, pro162, pro163, pro164, pro165, pro166, pro167, pro168, pro169, pro171, pro172, pro173, pro174, pro175, pro176, pro177, pro178, pro179, pro180, pro181, pro182, pro183, pro184, pro185, pro187, pro189, pro191, pro193, pro194, pro95, pro196, pro197, pro198, pro199, pro201, pro202, pro203, pro204, pro205, pro206, pro207, pro208, pro209, pro210, pro211, pro212, pro213, pro215, pro216, pro217, pro219, pro220, pro221, pro222, pro223, pro224, pro225, pro226, pro227, pro228, pro229, pro230, pro231, pro232, pro233, pro234, pro235, pro236, pro237, pro238, pro239, pro240, pro241, pro242, pro243, pro244, pro245, pro246, pro247, pro248, pro249, pro250, pro251, pro253, pro254, pro255, pro256, pro257, pro258, pro259, pro260, pro262, pro263, pro264, pro267, pro268, pro269, pro270, pro271, pro272, pro273, pro275, pro277, pro278, pro279, pro280, pro282, pro283, pro284, pro285, pro286, pro287, pro289, pro290, pro291, pro292, pro293, pro294, pro295, pro296, pro297, pro298, pro299, pro300, pro301, pro302, pro303, pro304, pro305, pro306, pro307, pro308, pro309, pro301, pro311, pro312, pro313, pro315, pro316, pro317, pro318, pro319, pro320, pro321, pro322, pro323, pro324, pro326, pro327, pro328, pro329, pro330, pro331, pro332, pro333, pro334, pro335, pro336, pro337, pro338, pro339, pro340, pro341, pro344, pro346, pro347, pro348, pro349, pro352, pro353, pro354, pro355, pro356, pro358, pro359 and pro361.

Even more preferably, the first nucleic acid sequences are selected from the group consisting of pro221, pro210, pro71 pro41, pro30, pro2, pro209, pro14, pro4, pro8, pro246, pro16, pro27, pro5, pro49, pro19, pro140, pro139, pro207, pro81, pro273 and pro362 and fragments thereof.

The first nucleic acid sequence may also comprise more than one fragment of nucleotide sequences selected from the above-mentioned group.

It is also contained within the present invention that the first nucleic acid sequence further comprises nucleic acid sequences not natively associated therewith. The nucleic acid sequences not natively associated therewith may for example be a transcription factor binding sites, preferably one or more steroid hormone receptor binding sites.

In preferred embodiments of the present invention the first nucleic acid sequences may be any first nucleic acid sequence as outlined herein above.

In certain embodiments, nucleic acid sequences are stably integrated into the genome of the cell. This integration may be in the cognate location and orientation via homologous recombination (gene replacement) or it may be integrated in a random, non-specific location (gene augmentation). In further embodiments, the nucleic acid sequences may be stably maintained in the cell as a separate, episomal segment of DNA. Such nucleic acid segments or “episomes” encode sequences sufficient to permit maintenance and replication independent of or in synchronisation with the host cell cycle.

The targeting complex may in addition to a binding partner and a bioreactive species further comprise additional components. Additional components may for example be protective components.

When the bioreactive species is a nucleic acid the targeting complex may further comprise a protective capping, wherein said protective capping consists of nucleic acid sequences attached to the first and/or second nucleic acid sequences. The nucleic acid sequences with protective properties may for example comprise a modified nucleotide. The modified nucleotide may for example be modified with one or more amino acids, amine groups or biotin groups.

In one embodiment of the present invention the bioreactive species is a toxin. A toxin is any species which is toxic to a cell expressing the cell surface molecule. For example the toxin may be selected from the group consisting of ricin, diphteria toxin, pseudomonas exotoxin, streptozotocin or cholera toxin. However, this list of toxins is not complete and should not be regarded as limiting to the invention.

In another embodiment of the present invention the bioreactive species is an inducers of apoptosis. Any compound, which is capable of inducing apoptosis directly or indirectly, in a cell expressing a cell surface molecule, is an inducer of apoptosis within the meaning of the present invention.

An inducer of apoptosis may be a polypeptide (see herein below) or it may be any other kind of compound. For example the inducer of apoptosis may be selected from the group consisting of retinoic acid, A23187, Okadaic Acid, Puromycin, Staurosporine, Thapsigargin, Actinomycin D, Camptothecin, Cycloheximide, Dexamethasone, Etoposide and Glucocorticoid. However, any other inducer of apoptosis is also contained within the present invention.

In yet another embodiment of the present invention the bioreactive species is a radioactive isotope. A radioactive isotope may be selected from the group consisting of (125)I, (131)I, (123)I, (111)In, (205)Bi, (206)Bi, (213)Bi, (186)Re, (188)Re, (225)Ac, 99 mTc, (68)Ga, (62)Cu, (90)Y, (64)Cu, (211)At, (212)Bi, (177)Lu, (153)Sm and (157)Gd. In one embodiment, the radioactive active species may be covalently 25 linked to another species, for example the radioactive species may be covalently linked to a binding partner.

In a still further embodiment of the present invention the bioreactive species is a cytostatica. A cytostatica may for example be a drug, which can be used for chemotherapy. Drugs suitable for use in chemotherapy are mentioned herein below.

The bioreactive species according to the present invention may be an antagonist of a hormone, preferably an antagonist of a hormone selected from the group consisting of estrogens, androgens, progesterones, LH and RH.

Androgens can for example be selected from the group consisting of testosterone, dihydrotestosterone, androstenediol, androstenedione, dehydroepiandrosterone (DHEA), dehydroepiandrosterone sulfate (DHEA-S) and derivatives thereof.

Estrogens can for example be selected from the group consisting of estrion, estradiol, estriol and derivatives thereof.

Alternatively, the bioreactive species may be an aromitase inhibitor.

In one preferred embodiment of the present invention the bioreactive species comprises or essentially consists of a polypeptide. In particular such a polypeptide may be a therapeutic protein.

The term “therapeutic protein” is intended to refer to any polypeptide introduced into a cell for the potential benefit of the cell or to an organism comprising said cell. A therapeutic protein may belong to a number of different classes. For example a therapeutic protein may be a tumour suppressor, a toxic substance or it may be an inducer of apoptosis. The therapeutic protein according to the present invention may be a protein, which can contribute to a cell cycle arrest.

In the context of cancer treatment modalities, a particularly useful gene is a tumour suppressor. During the process of transformation of normal cells to neoplastic cells, the mutation of tumour suppressor genes is thought to play an important role. One of the most important functions of a tumour suppressor gene is to attenuate cell division and mediate apoptosis of mutated cells. Tumour suppressor genes are highly effective, so that mutation of both alleles of the tumour suppressor gene is necessary to obviate its function. The introduction of a functional tumour suppressor gene into a cancer cell with a mutated phenotype is therefore often sufficient to induce cell cycle arrest and apoptosis. p53, p73 and p16 are tumour suppressor genes frequently mutated in lung cancer. Introduction of a wild type version of these genes into cancer cells using a therapeutic gene-delivering vector to induce apoptosis is a possible way to kill cancer cells selectively. There are numerous tumour suppressors well known to those in the art, preferred examples include p53, p73, p16, Rb, APC, DCC, NF-1, NF-2, WT-1, MEN-I, MEN-II, BRCA1, VHL, FCC and MCC. This list is not intended to be exhaustive of the various tumour suppressors known in the art but, rather, is exemplary of the more common tumour suppressors.

Preferably, the therapeutic protein is a tumour suppressor selected from the group consisting of p73, p16, Rb, APC, DCC, NF-1, NF-2, WT-1, MEN-1, MEN-II, BRCA1, VHL, FCC, MCC, MSH2, PTCH, DPCH, TSC2, CDKN2A and ARF. More preferably, the therapeutic protein is p53.

The important endpoint of therapy for cancer is the killing or elimination of cancer cells. One of the commonly used approaches for induction of this event is the introduction of wild type p53 into cancer cells with mutated p53, resulting in cell cycle arrest and induction of apoptosis. The use of p53 as a therapeutic gene is dependent on the status of the endogenous p53 in the cancer cell. Wild type overexpression is often efficient, however, overexpression of p53 in: combination with overexpression of cell cycle regulating genes, such as p16, may enhance the effect. Other cell cycle regulating genes such as p15, p17, p18 or p19 may also be effective in combination with p53 or other genes from the p53 family, such as p73. It is also possible that combination therapy with chemotherapeutic drugs or ionising radiation can markedly augment the therapeutic response to p53 gene therapy.

The Bcl-2 family of proteins are important regulators of cell death. They are comprised of two opposing factions, the proapoptotic versus the antiapoptotic members.

All bcl-2 family members share one or more of four highly conserved domains, BH1, BH2, BH3 and BH4. Bcl-2 family members include, but are not limited to, Al, mcl-1, bcl-2, bcl-w, bcl-x, bax, bad and bak A1, bcl-2, mcl-1, bcl-w and bcl-xl (a long form of bcl-x) genes encode intracellular membrane proteins shown to block or delay apoptosis. Overexpression of these genes has been shown to confer resistance to apoptosis including that induced by chemotherapy. Antisense oligonucleotides or ribozymes directed against these genes and their proteins can be used therapeutically to induce apoptosis.

In contrast, bax, bad, bak and bcl-xs (a short form of bcl-x) are presently known to promote cell death by inhibiting the protective effects of the antiapoptotic bcl-2 family members. A possible method of inducing apoptosis in tumour cells is by introduction and overexpression of these genes.

Caspases (cysteine-aspartic-acid-proteases) are a class of proteins central to the apoptotic program. These proteases are primarily responsible for the degradation of cellular proteins that lead to the morphological changes seen in cells undergoing apoptosis. Caspases are present as inactive pro-enzymes that are activated by proteolytic cleavage. At least 12 caspases has been identified in humans. Caspases 8, 9 and 3 are situated at pivotal junctions in apoptosis pathways. Caspase 8 and caspase 9 activate caspase 3 by proteolytic cleavage and caspase 3 then cleaves vital cellular proteins or other caspases. It is contemplated that the introduction and overexpression of one of these caspases will lead to apoptosis in cancer cells.

Preferably, the therapeutic protein is an inducer of apoptosis selected from the group consisting of Fas/Apol, TNF, TRAIL, TGF-β, caspases, Bak, Bax, Bid, Bik and GZMB.

The bioreactive species according to the present invention may furthermore be an antibody that bind oncogenic proteins or other proteins involved in the formation of cancer. A list of oncogenic protein are given herein above.

Cancer cells often produce growth factors and growth factor receptors to sustain autocrine or paracrine loops that mediate proliferation, angiogenesis and evasion of the immune system. Accordingly, the bioreactive species may be an antibody, for example an intracellular single chain that inhibits one or more growth factors selected from the group consisting of TGF-β, VEGF, IGF and growth factor receptors such as EGFR.

Additionally the therapeutic protein may be a protein capable of protecting the cell against a toxic agent or it may be a protein which is capable of catalysing the synthesis of a toxic substance.

Different systems have been developed where a protein is introduced that mediates the conversion of a prodrug to a cytotoxic compound. The herpes simplex virus thymidine kinase (HSV-tk) gene converts specific protoxic nucleoside analogs such as acyclovir and gancyclovir into potent DNA synthesis inhibitors. Cells, capable of expressing HSV-tk are rendered extremely sensitive to the drug, while non-HSV-tk expressing cells are relatively insensitive. The effects of the prodrug conversion is not only seen in the HSV-tk transduced cell, but also in the surrounding cells. This effect is termed the bystander effect, which is a therapeutic advantage, as it avoids the need to transduce 100% of the tumour cells with the HSV-tk gene.

Another such drug susceptibility therapeutic protein is the cytosine deaminase (CD). The CD protein catalyses the conversion of the prodrug 5-fluorocytosine (5FC) to 5-fluorouracil (5FU); treatment of CD transduced cells with 5FC results in the conversion of the 5FC into the antitumour drug 5FU into CD-positive tumour cells.

The therapeutic protein may furthermore be a toxic protein, such as cytokines, to be introduced to interfere with the expression of oncogenes and thus inhibit neoplastic cell growth.

The targeting complex according to the present invention may comprise more than one different bioreactive species, such as 2, for example 3, such as 4, for example 5, such as more than 5 different bioreactive species. For example the targeting complex may comprise more than one first nucleotide sequence encoding a therapeutic protein or more than one therapeutic protein, for example 2, such as 3, for example 4, such as 5, for example more than 5 first nucleotide sequences encoding a therapeutic protein and/or therapeutic proteins.

In some embodiments of the present invention the targeting complex further comprises a nuclear targeting signal. The nuclear targeting signal directs translocation into the nucleus. Certain bioreactive species must enter the nucleus to be active and accordingly it is advantageous if they are attached to a nuclear localisation signal. For example DNA sequences must enter the nucleus in order to be transcribed.

The nuclear targeting signal according to the present invention may be any nuclear targeting signal, which is capable of localising to the nucleus. The nuclear targeting signal may for example be an oligopeptide, preferably the nuclear targeting signal is selected from the group consisting of oligopeptide with the following sequences:
SEQ ID 310,
SEQ ID NO 311,
SEQ ID NO 312,
SEQ ID NO 313,
SEQ ID NO 314,
SEQ ID NO 315,
SEQ ID NO 316,
SEQ ID NO 317,
SEQ ID NO 318,
SEQ ID NO 319,
SEQ ID NO 320,
SEQ ID NO 321,
SEQ ID NO 322,
SEQ ID NO 323,
SEQ ID NO 324,
SEQ ID NO 325,
SEQ ID NO 326,
SEQ ID NO 327,
SEQ ID NO 328,
SEQ ID NO 329,
SEQ ID NO 330,
SEQ ID NO 331,
SEQ ID NO 332,
SEQ ID NO 333,
SEQ ID NO 334,
SEQ ID NO 335,
SEQ ID NO 336,
SEQ ID NO 337,
SEQ ID NO 338,
SEQ ID NO 339,
SEQ ID NO 338,
SEQ ID NO 340,
SEQ ID NO 341,
SEQ ID NO 342,
SEQ ID NO 343,
SEQ ID NO 344,
SEQ ID NO 345,
SEQ ID NO 346,
SEQ ID NO 347,
SEQ ID NO 348,
SEQ ID NO 349,
SEQ ID NO 350,
SEQ ID NO 351,
SEQ ID NO 352,
SEQ ID NO 353,
SEQ ID NO 354,
SEQ ID NO 355,
SEQ ID NO 356,
SEQ ID NO 357,
SEQ ID NO 358,
SEQ ID NO 359,
SEQ ID NO 360,
SEQ ID NO 361,
SEQ ID NO 362,
SEQ ID NO 363,
SEQ ID NO 364,
SEQ ID NO 365,
SEQ ID NO 366,
SEQ ID NO 367,
SEQ ID NO 368,
SEQ ID NO 369,
SEQ ID NO 370,
SEQ ID NO 371,
SEQ ID NO 372,
SEQ ID NO 373,
SEQ ID NO 374,
SEQ ID NO 375,
SEQ ID NO 376,
SEQ ID NO 377,
SEQ ID NO 378,
SEQ ID NO 379,
SEQ ID NO 380,
SEQ ID NO 381,
SEQ ID NO 382,
SEQ ID NO 383,
SEQ ID NO 384,
SEQ ID NO 385,
SEQ ID NO 386,
SEQ ID NO 387,
SEQ ID NO 388,
SEQ ID NO 389,
SEQ ID NO 390,
SEQ ID NO 391,
SEQ ID NO 392,
SEQ ID NO 393,
SEQ ID NO 394,
SEQ ID NO 395,
SEQ ID NO 398,
SEQ ID NO 397,
SEQ ID NO 398,
SEQ ID NO 399,
SEQ ID NO 400,

Wherein the name of the protein from which they have been derived is indicated in brackets and wherein ‘[KR]’ indicates ‘K or R’, i.e. any of the two amino acids valid at that position, ‘x’ for ‘any amino acid’, ‘x{9}’ for ‘9 times x’, and ‘x{7,9}’ for ‘at least 7, at most 9 times x’. Amino acids are given in their one-letter code.

Furthermore, nuclear localisation signal according to the present invention may also be mutants of the above mentioned sequences, such as mutants wherein 1, such as 2, for example 3, such as 4, for example 5, such as 6, for example 7, such as 8, for example 9, such as 10 amino acids have been substituted for any another amino acid, preferably it is a conservative amino acid substitution (see herein above). Mutants wherein 1, such as 2, for example 3, such as 4, for example 5, such as 6, for example 7, such as 8, for example 9, such as 10 amino acids have been deleted are nuclear localisation signal according to the present invention.

More preferably, the nuclear targeting signal is the nuclear localisation signal of simian virus 40 large tumour antigen.

In certain embodiments of the present invention the targeting complex further comprises a endosomal lytic agent. The targeting complex is frequently taken up into cells expressing the cell surface molecule by a process known as receptor mediated endocytosis and accordingly the targeting complex enters the cell in an endosome, which it has to escape in order to avoid degradation. Hence, the targeting complex often comprise an endosomal lytic agent.

Many viruses have developed strategies to escape the endosome and accordingly an attenuated virus or parts of a virus may be useful endosomal lytic agents. Preferably, the endosomal lytic agent is selected from the group consisting of polyethylenimine (PEI), a replication defective virus and a viral protein capside. More preferably, the endosomal lytic agent may comprise a membrane destabilising, polypeptide.

In one embodiment of the present invention the targeting complex further comprises chloroquine. Chloroquine may protect against endosomal degradation and its presence is accordingly desirable in some embodiments of the invention.

In preferred embodiments of the present invention the bioreactive species and the binding partner associates with one another either directly or indirectly. If the bioreactive species is a nucleic acid sequence, the binding partner may for example associate with the bioreactive species via a nucleic acid binding agent covalently attached to said binding partner.

Nucleic acid-binding agents include proteins, polypeptides, peptides, antibodies, nucleotides, carbohydrates, fatty acids, organic or inorganic compounds as well as a combination of these and others.

Nucleic acid-binding agents may bind to single-stranded or double-stranded DNA, to single-stranded or double stranded RNA, by chemical or physical forces or by a combination of the two. A nucleic acid-binding agent may (i) have affinity only for the nucleic acid itself, (ii) have affinity for both the nucleic acid and another molecule, thereby forming a bridge between the two or (iii) have indirect affinity for the nucleic acid via affinity for another molecule that has affinity for the nucleic acid.

According to the present invention, the coupling of a nucleic acid-binding agent and the binding partner must occur in a manner that does not interfere with the binding of the binding partner with the cell surface molecule. Preferably, internalisation of the targeting complex via receptor-mediated endocytosis is also retained. In an even more preferred embodiment, this recognition and internalisation delivers the, nucleic acid sequences into a target cell in a form suitable for the expression or for interaction with target endogenous nucleic acid.

In one embodiment, the nucleic acid-binding agent may insert itself between base pairs of double-stranded nucleic acids in an intercalative manner or bind in the minor or major groves of double-stranded nucleic acids.

This binding may be sequence-specific or completely unrelated to sequence. In other embodiments, nucleic acids may be cross-linked with other molecules with chemically or photochemically reactive groups.

In another embodiment of the invention, the nucleic acid-binding agent covalently links the nucleic acid to another molecule. In one embodiment, the nucleic acid binding agent is one of the coupling agents, such as carbodiimide. However, covalent coupling of the nucleic acid may alter its specificity and preclude proper gene expression or target nucleic acid recognition. Furthermore, linear or single stranded nucleic acid may be a requirement for covalent coupling of the nucleic acid to the binding partner. Finally, nucleic acids are negatively charged molecules which means that they may be repelled from cell surfaces, making transfer difficult via the endosomal lysis pathway. Therefore, a size and type restriction may be necessary for the efficient delivery of nucleic acid directly bound to binding partner.

An example of a nucleic acid-binding agent, is a polycationic agent that depends on electrostatic-dominated binding involving sequence-neutral interactions between the cationic groups and the negatively charged phosphates on nucleic acid similar to the DNA-binding agent described in WO 96/30536.

The polycationic agent binds DNA strongly resulting in the formation of a toroid complex where the negative charge of nucleic acid molecule is completely neutralised. This soluble toroid complex may be internalised via normal receptor-mediated endocytosis.

Any type of nucleic acid may be used, from single stranded mRNA to double stranded circular plasmids.

Furthermore, any size of nucleic acid may be used, as long as there is a source of negative charge for the polycationic agent to bind. In certain embodiments, these polycationic moieties may include a natural polyamine such as spermine and/or spermidine. In a preferred embodiment, the polycationic agent may be an artificially produced agent, such as polylysine or polyethyleneimine.

In order for the invention to function properly, certain criteria with regard to the nucleic acid-binding agent need to be fulfilled. First, the nucleic acid to be delivered into the cell must bind to the nucleic acid binding agent without loosing its integrity in any way.

Secondly, the complex comprising of ligand, nucleic acid binding agent and nucleic acid must be in soluble form to allow greater accessibility of the complex to, cells in vitro and in vivo. Thirdly, once the complex is internalised within the host cell, the nucleic acid must have access to its target sequence while avoiding degradation.

The nucleic acid binding agent may include agents such as carbodiimides, N-succinimidyl, 3 (2-pyridyldithio) propionate, succinimmidyl, 4-(N-maleimidomethyl) cyclohexane-1-carboxylate, diisocyanates, glutaraldehyde, diazobenzenes, and hexamethylene diamines. This list is not intended to be exhaustive of the various coupling agents known in the art but, rather, is exemplary of the more common linking agents that may be used.

Preferably, the nucleic acid binding agent is selected from the group consisting of poly-L-lysine (PLL), spermine, spermidine and histone proteins.

When the nucleic acid binding agent is PLL, PLL may be comprising from 15 to 1000, such as from 50 to 750, for example from 100 to 500, such as from 200 to 400 residues.

In one embodiment of the present invention the binding partner associates with the bioreactive species indirectly via a pair of specific interacting components wherein one component is covalently attached to the bioreactive species and the second component is covalently attached to the binding partner.

One example of such a pair of specific interacting components is biotin and streptavidin, however other pairs of interacting components may also be used.

Complex Comprising Cell Surface Molecule and Targeting Complex

It is one objective of the present invention to provide complexes that comprise a cell surface molecule, a binding partner and a bioreactive species. Example of cell surfaces molecules, binding partner and bioreactive species are given herein above.

Preferably, the complex may comprise a cell surface molecule identified according to any of the methods according to the present invention and a targeting complex as described herein above.

Alternatively, the complex may comprise a cell surface molecule and a targeting complex as described herein above, wherein said cell surface molecule preferably comprises or essentially consists of or for example is a cell surface molecule mentioned in table 2.

More preferably, the cell surface molecule may be selected from the group consisting of NCAM1, NPTXR, LRP8, CHRNA5, GRIA2, GRM8, ITGAV, ITGAE, TNFRSF12, L1CAM, GPR49 and TMEFF1.

Conditions

A premalignant and/or malignant conditions may for example be cancer or a conditions which may develop into a cancer. The term cancer within the scope of the present invention covers both malignant and benign tumours, as well as leukaemia.

Cancer may for example be adenomas, carcinomas or sarcomas. Cancer may for example be selected from the group consisting of melanoma, brain tumours, neuroblastomas, breast cancer, lung cancer, prostate cancer, cervix cancer, uterine cancer, ovarian cancer, leukaemia, colon cancer, rectum cancer, cancer of the testis, cancer of the kidney, cancer of the liver, cancer of the lip, cancer of the tongue, cancer of the stomach, skin cancer, sarcomas, mesotheliomas, bladder cancer, bone tumours, malignant pleural effusions, ascites, meningeal carcinomatosis, head and neck cancers and cancers of endocrine organs such as: thyroid gland, pituitary gland and suprarenal gland.

Lung cancer may for example be cancers selected from the group comprising small cell lung cancer (SCLC) and non-small cell lung cancer (NSCLC). Preferably, the premalignant and/or malignant conditions is small cell lung cancer.

In one preferred embodiment the premalignant and/or malignant conditions is breast cancer.

In another preferred embodiment the premalignant and/or malignant conditions is a brain tumour. Brain tumours may for example be selected from the group comprising glioblastomas, neuroblastomas, astrocytomas, oligodendrogliomas, meningiomas, medulloblastomas, neuronomas, ependymomas, craniopharingiomas, pineal tumours, germ cell tumours and schwannomas.

Administration and Pharmaceutical Compositions

The individual to receive treatment is any animal, however, preferably the individual is a human being.

The treatment according to the present invention may be ameliorating treatment, it may be curative treatment and/or it may be prophylactic treatment.

The main routes of drug delivery according to the present invention are intravenous, oral and subcutaneous, as will be described below. Other drug-administration methods, such as topical delivery, which are effective to deliver the drug to a target site or to introduce the drug into the bloodstream, are also contemplated. The compounds may also be administered by inhalation, that is by intranasal and oral inhalation administration.

The mucosal membrane to which the pharmaceutical preparation of the invention is administered may be any mucosal membrane of the mammal to which the biologically active substance is to be given, e.g. in the nose, vagina, eye, mouth, genital tract, lungs, gastrointestinal tract, or rectum.

Compounds of the invention may preferably be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques.

Preferably, the targeting complex according to the present invention is administrated parenterally, more preferably the targeting complex is administrated by intravenous injection and/or by subcutaneous injection.

The compounds according to the invention may be administered with at least one other compound. The compounds may be administered simultaneously, either as separate formulations or combined in a unit dosage form, or administered sequentially.

The dosage requirements will vary with the particular drug composition employed, the route of administration and the particular individual being treated. Ideally, an individual to be treated by the present method will receive a pharmaceutically effective amount of the compound in the maximum tolerated dose, generally no higher than that required before drug resistance develops.

The individual dosages of a targeting complex will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on interindividual differences in pharmacokinetics, drug distribution, and metabolism. The “effective level” can be defined, for example, as the blood or tissue level desired in the individual that corresponds to a concentration of one or more compounds according to the invention.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

Pharmaceutical acceptable salts of the compounds according to the present invention should also be considered to fall within the scope of the present invention. Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, In an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, ptoluenesulphonic acids, and arylsulphonic, for example.

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefor.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilisers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilised by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations, which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilising and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilisers, buffers, artificial and natural sweeteners, dispersants, thickeners, solubilising agents, and the like.

The compounds of the present invention may be formulated for parenteral administration (e.g., by injection, for example bolus injection or continuous infusion) and may be presented in unit dose form in ampoules, pre-filled syringes, small volume infusion or in multidose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilising and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturiser such as glycerol or an oil such as castor oil or arachis oil.

The pharmaceutical active compound described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the active compound to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical active compound in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient.

Pharmaceutical compositions usually comprise a carrier. Illustrative solid carrier include lactose, terra alba, sucrose, talc, gelatin, agar, pectin, acacia, magnesium stearate, stearic acid and the like. A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions, and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Illustrative liquid carriers include syrup, peanut oil, olive oil, water, etc. Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilisers, emulsifiers, buffers, preservatives, sweeteners, flavouring agents, suspending agents, thickening agents, colours, viscosity regulators, stabilisers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carders are useful in sterile liquid form compositions for parenteral administration. The liquid carrier for pressurised compositions can be halogenated hydrocarbon or other pharmaceutically acceptable propellant. Liquid pharmaceutical compositions which are sterile solutions or suspensions can be utilised by, for example, intramuscular, Intraperitonealior subcutaneous injection. Sterile solutions can also be administered intravenously. The compound can also be administered orally either in liquid or solid composition form.

The carrier or excipient may include time delay material well known to the art, such as glyceryl monostearate or glyceryl distearate along or with a wax, ethylcellulose, hydroxypropylmethylcellulose, methylmethacrylate and the like. When formulated for oral administration, 0.01% Tween 80 in PHOSAL PG-50 (phospholipid concentrate with 1,2-propylene glycol, A. Nattermann & Cie. GmbH) has been recognised as providing an acceptable oral formulation for other compounds, and may be adapted, to formulations for various compounds of this invention.

Combination Therapies

The targeting complex according to the present invention may be administrated I combination with one or more second treatments, for example treatments which are currently used to treat cancer.

For example such second treatments may be selected from the group consisting of surgical treatment, chemotherapy, radiation therapy, therapy with cytokines, Hormone therapy, gene therapy, immunotherapy and treatments using laser light.

Chemotherapy comprise administration of a chemotherapeutical agent, such as a cytostatica. Cytostatica according to the present invention may for example be selected from the group consisting of carboplatin, cisplatin, cyclophosphamide, iphosphamide, hexamethylmelamine, doxorubicin, epirubicin, etopiside (VP-16), teniposide (VM-26), vincristine, vindecine, taxans, irinotecan, tyrosin kinase inhibitors, nimustine, lomustine, BCNU, farnesyl transferase inhibitors anti angiogenestic compounds, anti metastatic compounds, 5-fluoruracil±leucovorin, topoisomerase inhibitor I and II and Temozolamide.

In addition, chemotherapy may for example comprise administration of Antiestrogen, Anti-progesteron, anti-androgen, LH-RH antagonists or aromatase inhibitors

### EXAMPLES

The following are examples of embodiments of the invention and should not be regarded as limiting for the present invention.

### Example 1

Culture of Small Cell Lung Cancer (SCLC) Cell Lines:

The following small cell lung cancer cell lines were used for analysis

| | | Growth: | |
|---|---|---|---|
| | | A = adherent | |
| Cell line | Cell line established | S = suspension | Growth medium |
| CPH 54A | University of Copenhagen, | A | MEM (EAGLE) + 10% FCS |
| CPH 54B | Denmark (Engelholm et al., | A | MEM (EAGLE) + 10% FCS |
| | 1986) | | |
| GLC 2 | Groningen Lung Cancer Centre, | A (S) | RPMI + 10% FCS |
| GLC 3 | The Netherlands (de Leij et | S (A) | RPMI + 10% FCS |
| GLC 14 | al., 1986; Berendsen et al., | S | RPMI + 10% FCS |
| GLC 16 | 1988, Bulte et al., 1993) | S | RPMI + 10% FCS |
| GLC 19 | | S | RPMI + 10% FCS |
| GLC 26 | | S | RPMI + 10% FCS |
| GLC 28 | | S | RPMI + 10% FCS |
| DMS 53 | Dartmouth Medical School, | A | Waymouth + 10% FCS |
| DMS 79 | NH, USA (Pettengill et al., | S | RPMI + 10% FCS |
| DMS 92 | 1980) | A (S) | Waymouth + 10% FCS |
| DMS 114 | | A | Waymouth + 10% FCS |
| DMS 153 | | A | Waymouth + 10% FCS |
| DMS 273 | | A | Waymouth + 10% FCS |
| DMS 406 | | A (S) | Waymouth + 10% FCS |
| DMS 456 | | A (S) | Waymouth + 10% FCS |
| NCI H69 | National Cancer Institute, | S | RPMI + 10% FCS |
| NCI N417 | MD, USA (Carney et al., 1985) | S | RPMI + 10% FCS |
| MAR H24 | Philips University, Marburg, | S | RPMI + 10% FCS |
| MAR 86 MI | Germany (Bepler et al., 1987) | S | RPMI + 10% FCS |

All cells were maintained at 37° C. in 5% CO₂ in a humidified atmosphere in medium without antibiotics and passaged twice weekly. All media and serum were obtained from Life Technologies.

Xenografts

0.5-1.2×10⁷ cells were inoculated bilaterally, subcutaneously in the flanks of 12-13 weeks old Balb/c nude mice. The mice were sacrificed and the xenografted tumors were harvested when one of the tumors had reached a maximal diameter of 1 cm. Necrotic tissue was removed. The cell line CPH 136A was only propagated in nude mice by inoculation of a 2 mm tumor block. Tumors for RNA isolation were either processed immediately or stored 24 hours in RNA later (Ambion) followed by storage at −70° C. and processed as described below. Tumors used for lysates for Western blot analyses were processed immediately as described below.

RNA from Normal Tissues

Total RNA from normal, human tissues were obtained from either Clontech (fetal brain, brain, lung, kidney, heart, trachea, adrenal gland, prostate, salivary gland, thyroid) or from Ambion (lung, liver, brain, pancreas, spleen, small intestine, skeletal muscle, colon, stomach, testes). Only one sample was analysed in duplicate (lung RNA from Clontech and Ambion) and one in triplicate (brain RNA from 2 different batches from Clontech and one from Ambion). Fetal brain was included as a reference for embryonal, neuroendocrine tissue.

Isolation of RNA from Cell Lines.

Cells from semi-confluent cultures were harvested (by trypsinisation for adherent cells) and total RNA from approx. 10⁷ cells was isolated using RNeasy Kit (Qiagen) according to manufacturers instructions. Xenografted tumors (fresh or after storage in RNA later) were homogenised in TRIzol (Life Technologies) and RNA purified according to the manufacturers instruction. The TRIzol isolated RNA was further purified using RNAeasy kit (Qiagen).

The concentration of the RNA was estimated by the absorption at 260 nm (A₂₆₀). The integrity of the RNA was verified by measuring the ratio of A_{268/280} to be 1.9 or more and by estimating the ratio of 28S rRNA to 18S rRNA analysed by formaldehyde (denaturing) gel analysis to being approximately 2.

Preparation of cDNA.

10 μg total RNA in 10 μl H₂O was hybridised to 100 pmol T7-(dT)₂₄ primer (HPLC purified 5′-GGCCAGTGAATTGTAATACGACTCACTATAGGGAGGCGGT)₂₄ (SEQ ID NO 401) obtained from GENSET) after denaturation at 70° C. for 10 min. The following reactions were performed using reagents from Gibco BRL, Life Technologies. First strand synthesis was performed using 400 U SuperScript RnaseH⁻ Reverse Transcriptase kit in a 20 μl reaction in first strand buffer (50 mM Tris-HCl (pH 8.3), 75 mM KCl, 3 mM MgCl₂) 10 mM DTT, 0.5 mM dNTPs (each) at 42° C. for 1 hour. The second strand synthesis was performed in a 150 μl reaction in second strand buffer (20 mM Tris-Cl (pH 6.9), 5 mM MgCl₂, 100 mM KCl, 0.15 mM β-NAD⁺, 10 mM(NH₄)₂SO₄ containing 0.26 mM dNTPs, 0.07 U/μl E. coli DNA ligase, 0.27 U/μl E. coli DNA polymerase, 0.013 U/μl E. coli Rnase H by incubation for 2 hours at 16° C. DNA ends were filled out by addition of 0.07 U/μl T4 DNA polymerase and incubation for 5 min at 16° C. The reactions were terminated by addition of EDTA to 33 μM final concentration. The cDNA was purified by extraction with 1 volume phenol:chloroform:isoamylalchbhol (25:24:1) saturated with 10 mM Tris-HCl (pH 8.0), 1′ mM EDTA followed by precipitation in 2.5 M NH₄Ac in 63% ethanol with addition of 2 μl Pellet Paint (Novagen) for visualization of pellet. After 2 consecutive rinsing of the pellet with 80% ethanol, the pellet was air dried and dissolved in 12 μl water. An aliquot was analysed by agarose gel electrophoresis to ensure the length of the cDNA to be in the range of 0.1->10 kb.

Preparation of Biotin Labelled cRNA (IVT-cRNA)

In vitro transcription generating biotin labelled cRNA (complementary RNA) with T7 RNA polymerase using biotin labelled ribonucleotides was performed with the BioArray™, High Yield™RNA transcript labelling kit from Enzo Diagnostics, NY, USA) using 6 μl cDNA (estimated to contain approx. 1 μg cDNA) in a 40 μl reaction according to manufacturers specifications. The biotin labelled cRNA was purified using RNeasy spin columns kit (Qiagen) according to manufacturers specified method for RNA cleanup. An aliquot of the IVT-cRNA was analysed by denaturing agarose gel electrophoresis to ensure full length transcripts (0.1->10 kb). The concentration of the cRNA was estimated by the absorption at 260 nm and corrected for contribution of total RNA initially used for the cDNA reaction. The yield varied from 25-100 μg per reaction.

Fragmentation of IVT-cRNA

22 μg IVT-cRNA was fragmented by incubation in 0.04 M Tris-Acetate (pH 8.1), 0.03 M MgAc, 0.1 M KAc in a 20 μl reaction for 35 min at 94° C. An aliquot of the fragmented IVT-cRNA was analysed by agarose gel electrophoresis to ensure fragmentation to the size of 30-200 bases.

Hybridisation to Affymetrix GeneChip™ and Analysis of Data (CHIPs Analysis)

A hybridisation mixture containing 20 μg of fragmented IVT-cRNA in a volume of 400 μl containing 0.1 M MES, 0.75, [Na⁺], 0.1 mg/ml herring sperm DNA, 0.1 mg/ml acetylated BSA, 0.05 nM biotinylated control oligo B2 (5′-GTCGTCMGATGCTACCGTTCAGGA (SEQ ID NO 402)) and control biotin labelled IVT-cRNA for spiking prepared from the plasmids pglks-bioB (150 μM), pglks-bioC (500 μM), pglks-bioD (2.5 nM) and pglks-cre (10 nM) (American Tissue Culture Collection). The control oligo and control cRNAs were obtained from Affymetrix. 100 μl was hybridised to an Affymetrix test2 CHIP followed by staining with a streptavidin-phycoerythprn conjugate and labelling with biotinylated anti-streptavidin goat antibody followed by a final staining with streptavidin-phycoerythrin conjugate (according to the manufacturers protocol Mini-euk1) or 300 μl was hybridised to an Affymetrix U95A GeneChip and stained according to the manufacturers protocol Eu-kGE-WS2 in an Affymetrix Fluidics station and scanned at 560 nm in a confocal laser scanner (Hewlett Packard GeneArray Scanner G2500A). The digitalized image data was first processed using Affymetrix Microarray Suite™ version 4.0 for evaluation of the quality of the RNA and hybridisation and Affymetrix Data Mining Tool (version 2.0) for selection of candidate genes. The data was re-analysed using Affymetrix Microarray Suite™ version 5 (see results) for selection of surface molecules. Data was only used from analyses where: the control oligos BioB, BioC, BioD and Cre were all detected as present; the scaled noise (Q) was below 10; the ratio of detection of the mRNA levels of the 5′ ends relative to the 3′ end of glyceraldehyde-3-phosphate dehydrogenase (GAPDH) and beta-actin were below 2; at least 40% of all probe sets were identified as present. For comparison between samples, the global intensity was set at 100.

RT-PCR

Semi-quantitative RT-PCR was performed on selected genes for validation of the Chips analysis cDNA prepared as described above was used for the RT-PCR but as an independent preparation than used for Chips analysis. The PCR reaction was performed using cDNA from 350 ng total RNA in a 25 μl reaction with 200 nM primers (DNA Technology A/S), 1.5 mM MgCl₂, 0.2 mM each dNTPs, 0.1 U/μl Platinum Taq Polymerase (Life Technologies) in the buffer provided with the enzyme with 0.008% cresol red and 12% sucrose as loading buffer.

All reactions were run 94° C., 2 min, 1 cycle; 94° C., 30 sec, annealing temperature as indicated for each primer set, 30 sec, 72° C., 30 sec for 25 cycles and a final extension step of 72° C. 10 min. Using only 25 cycles makes the reaction semi-quantitative.

The Primer Sets Used Were:

Glyceraldehyde-3phosphate dehydrogenase (GAPDH)

512 bp PCR product spanning GenBank Acc. no. NM_{—}002046 bp. 608-1119,
Sense: 5′-TCCATGCCATCACTGCCACCCA (SEQ ID NO 403)
Antisense: 5′-TCTTGTGCTCTTGCTGGGGCTG (SEQ ID NO 404) Annealing temp. 56° C.
   One RT-PCR reaction has been performed.
   Pro 30 (KIAA0042)

432 bp PCR product spanning GenBank Acc. no. D26361 bp. 5181-5612
Sense: 5′-GTTTTGAATCTGAAGAAAGCCC (SEQ ID NO 405)
Antisense: 5′-TCAAACTCCTGACCTTGTGATCT (SEQ ID NO 406) Annealing temp. 49° C.

2 independent RT-PCR reactions have been performed.

Pro 41 (MAD2)

525 bp PCR product spanning GenBank Acc no. AJ000186 bp. 643-1167,
Sense: 5′-GTAAATAGCATGGTGGCCTACA (SEQ ID NO 407)
Antisense: 5′-GGTCCAAAGGAGCTATACAGCA (SEQ ID NO 408) Annealing temp. 45° C.

2 independent RT-PCR reactions were performed.

Pro 221 (Insulinoma-Associated Antigen, IA-1)

532 bp PCR product spanning GenBank Acc. no. M93119 bp. 1549-2080,
Sense: 5′-GTGTTCCCCTGCAAGTACTGCCC (SEQ ID NO 409)
Antisense: 5′-CAGAGATTGGTAGGCGAGGCGA (SEQ ID NO 410) Annealing temp. 52° C.

2 independent RT-PCR reactions were performed.

Pro 210 (lamin B1)

439 bp PCR product spanning GenBank Acc. no. L37747 bp. 424-862,
Sense: 5′-ACTGTGTACTGTrCGGAAGGG (SEQ ID NO 411)
Antisense: 5′-TAGAGAAACCCTTCCCTCCC (SEQ ID NO 412) Annealing temp. 46° C.

Only one RT-PCR reaction has been performed. RT-PCR was not performed on testis

Pro 71(p16INK4/MTS 1, CDKN2A)

437 bp PCR product spanning GenBank Acc. no. U26727 bp.176-612.,
Sense: 5′-TGAGGAGCCAGCGTCTAGGG (SEQ ID NO 413)
Antisense: 5′-GTGGCCCTGTAGGACCTTCG (SEQ ID NO 414) Annealing temp. 57° C.

Only one RT-PCR reaction has been performed. RT-PCR was not performed on testis

DR6 (TNFRS12, Tumor Necrosis Factor Receptor Superfamiliy Member 21)

559 bp PCR product spanning GenBank Acc. no. AF068868 bp. 1081-1639,
Sense: 5′-GTGCTTGTGGTGATTGTGGTGTG (SEQ ID NO 415)
Antisense: 5′-TGTTCTTGTCCTGTGGGGAAGG (SEQ ID NO 416) Annealing temp., 56° C.

2 independent RT-PCR reactions were performed.

NCAM1 (Neural Cell Adhesion Molecule)

456 bp PCR product spanning GenBank Acc. no. HSU63041 bp 2045-2500,
Sense: 5′-TATGAGGTCTACGTGGTGGC (SEQ ID. NO 417)
Antisense: 5′-CTCCTGGCACTCTGGCTTTG (SEQ ID NO 418) Annealing temp. 53° C.

Only one RT-PCR reaction has been performed. RT-PCR was not performed on testis

NPTXR (Neuronal Pentraxin Receptor)

482 bp PCR product spanning GenBank Acc. no. HS327J16 bp.46012-493,
Sense: 5′-CACACGCACACATGTTGCAGC (SEQ ID NO 419)
Antisense: 5′-GCTCTGAGAGGCCAAAGCC (SEQ ID NO 420) Annealing temp. 55° C.

Only one RT-PCR reaction has been performed. RT-PCR was not performed on testis

GLUR2 (Ionotropic Glutamate Receptor 2; GRIA2)

522 bp PCR product spanning GenBank Acc. no. L20814 bp. 2449-2970,
Sense: 5′-AGGAACCCCAGTAAATCTTGCAG (SEQ ID NO 421)
Antisense: 5′-TCAGTCACACTGACATTCATTCCC (SEQ ID NO 422) Annealing temp. 51° C.

Only one RT-PCR reaction has been performed. RT-PCR was not performed on testis

ITGAV (Integrin Alpha V Subunit)

533 bp PCR product spanning GenBank Acc. no. M14648 bp. 3867-4399,
Sense: 5′-AATTTTAGGTCAAATCCTTCMGCCAAC (SEQ ID NO 423)
Antisense: 5′-TGACAGCCGAGACTGATTTTACACATTA (SEQ ID NO 424) Annealing temp. 50° C.

Only one RT-PCR reaction has been performed. RT-PCR was not performed on testis

LRP8 (Apolipoprotein E Receptor 2)

459 bp PCR product spanning GenBank Acc. no. HSZ75190 bp. 2016-2474
Sense: 5′-GCTCCATATAGGGAGAACTGCTCAG (SEQ ID NO 425)
Antisense: 5′-CCCCAGCAACCAAACATCTTCT (SEQ ID NO 426) Annealing temp. 50° C.

Only one RT-PCR reaction has been performed. RT-PCR was not performed on testis.

Western Blotting

Protein Samples

Whole cell lysates were extracted from cell lines and xenografted tumors for validation of protein expression of selected genes. The lysates were prepared from semi confluent cultures of cell lines by scraping with a rubber policeman (for adherent cells) and washing in ice-cold 20 mM Tris-Cl pH 7.5. The cell pellet was lysed in ice cold 20 mM Tris-Cl pH 7.5, 2% Triton X-100 containing Protease Inhibitor Cocktail set 11 and III (Calbiochem) diluted 1:100. After vortexing the lysates were cleared by centrifugation at 15.000×g for 5 min, 4° C. Lysates from xenografted tumors was prepared immediately after harvesting of the tumors and a lysate from an adult rat brain was processed in a similar manner. The tumors were weighed and homogenised using a Heindolph DIAX 900 homogenised in 5 volumes (w/w) ice cold 20 mM Tris-Cl pH 7.5, 2% Triton X-100 containing Protease Inhibitor Cocktail set 11 and III (Calbiochem) diluted 1:100. cleared by centrifugation at 15.000×g for 5 min, 4° C. Protein concentration of the lysates was determined using the BCA Protein Assay (Pierce) as recommended by the manufacturer.

Commercial cell lysates of Jurkat (Santa Cruz) and A431 (Neomarkers) were used as positive controls in some western blots.

SDS-PAGE and Blotting.

5-15 μg lysate was loaded per lane in LDS sample buffer with reducing agent (NuPAGE) and separated on 3-8% Tris Acetate SDS gels, run for 150 V 1 hr in Tris-Acetat SDS running buffer (NuPAGE) and transferred to PVDF LC 2002 (Novex) membrane in Transfer Buffer (NuPAGE). Protein size marker was ProSieve colour protein marker. For probing with anti-NCAM1 antibodies, the lysates were pretreated for 5 min at 37° C. with 40 ng/μl recombinant EndoN-HIS (gift from E. Bock) to remove polysialylation.

The membranes were blocked in washing buffer (10 mM Tris-Cl pH 7.5, 100 mM NaCl, 0.1% Tween 20) containing 5% low fat milk for 60 min at room temperature (for antibodies against Integrin αE (CD103), For ITGAE a Tris-Cl buffer pH 10.2 was used for all incubation and washing procedures. The blots were incubated with primary antibodies and secondary antibodies in blocking buffer as described below and bound antibodies visualised by ECL (Amersham) or alkaline phosphatase using NBT/BCIP tablets (Roche) as recommended by the manufacturers.

NCAM1 (Neural Cell Adhesion Molecule)

Primary antibody: Mouse monoclonal anti-NCAM1 clone 123C3 (Santa Cruz) diluted 1:100 Incubation 16 hours at 4° C. Secondary antibody: Alkaline phosphatase conjugated rabbit anti-mouse Ig (DAKO) diluted 1:500. Incubation 1 hour at room temperature. Development by alkaline phosphatase.

GluR2 (Ionotropic Glutamate Receptor 2)

Primary antibody: Mouse monoclonal anti GluR2 and 4 (clone3A11) (Pharmingen) diluted 1:500 Incubation 16 hours at 4° C. Secondary antibody: Alkaline phosphatase rabbit anti mouse Ig (DAKO) diluted 1:500 Incubation 1 hour at room temperature. Development by alkaline phosphatase.

GRM8 (GluR8 (Metabotropic Glutamate Receptor 8))

Primary antibody: Rabbit polyclonal anti-mGluR8 (Upstate Biotechnology, TriChem) diluted 1:500. Incubation 16 hours at 4° C. Secondary antibody: Horseradish peroxidase swine anti rabbit 1 g (DAKO) diluted 1:1000 Incubation 1 hour at room temperature. Development by ECL.

NPTXR (Neuronal Pentraxin Receptor)

Primary antibody: Goat polyclonal anti NPTXR (C-17)(Santa Cruz) diluted 1:500 Incubation 16 hours at 4° C. Secondary antibody: Horseradish peroxidase rabbit anti goat Ig (DAKO) diluted 1:1000 Incubation 1 hour at room temperature. Development by ECL

ITGAE (Integrin Alpha E Subunit)

Primary antibody: Goat polyclonal anti Integrin αE (N-19) (Santa Cruz) diluted 1:1000. Incubation 16 hours at 4° C.

Secondary antibody: Alkaline phosphatase rabbit anti goat Ig (sc-2771) (Santa Cruz) diluted 1:500. Incubation 1 hour at room temperature

Development by alkaline phosphatase.

Cluster Analysis of Obtained Chips Analysis Data.

To interpret the variation in expression patterns seen between the normal tissues analysed and the small cell ling cancer cell lines we took advantage of the properties of both SOMs (self-organising maps) and hierarchical clustering. These were used consecutively to group genes on the basis of similarity in the pattern of expression. Genes used for the analysis were those that had an average difference of more than 50 and were scored present in any one of the samples.

SOMs

Self-Organising maps (SOMs) is a method of cluster analysis that is somewhat related to k-means clustering. The basic principle behind the SOM algorithm is that the weight vectors of neurons, which are first initialised randomly, come to represent a number of original measurement vectors during an iterative data input (Toronen et al, 1999). The following parameters were used in the calculations: Genes: Xdim: 1, Ydim: 10, Iterations: 100000, Samples: Xdim: 1, Ydim: 10, Iterations: 20000.

Hierarchical Clustering

The basic idea behind hierarchical clustering is to assemble a set of items (genes or arrays) into a tree, where items are joined by very short branches if they are very similar to each other, and by increasingly longer branches as their similarity decreases. The output file from the SOM clustering is used for the hierarchical clustering, meaning that the ordering by the SOM clustering is used to guide the flipping of nodes in the hierarchical tree (Eisen et al., 1998). The following parameters were used in the calculations: Genes: Cluster Yes, Calculate weights: Yes, Similarity matrix: correlation uncentered, Samples: Cluster: Yes, Calculate weights: Yes, Similarity matrix: correlation uncentered. Subsequently an Average Linkage cluster analysis was performed.

Results of Clustering Analysis

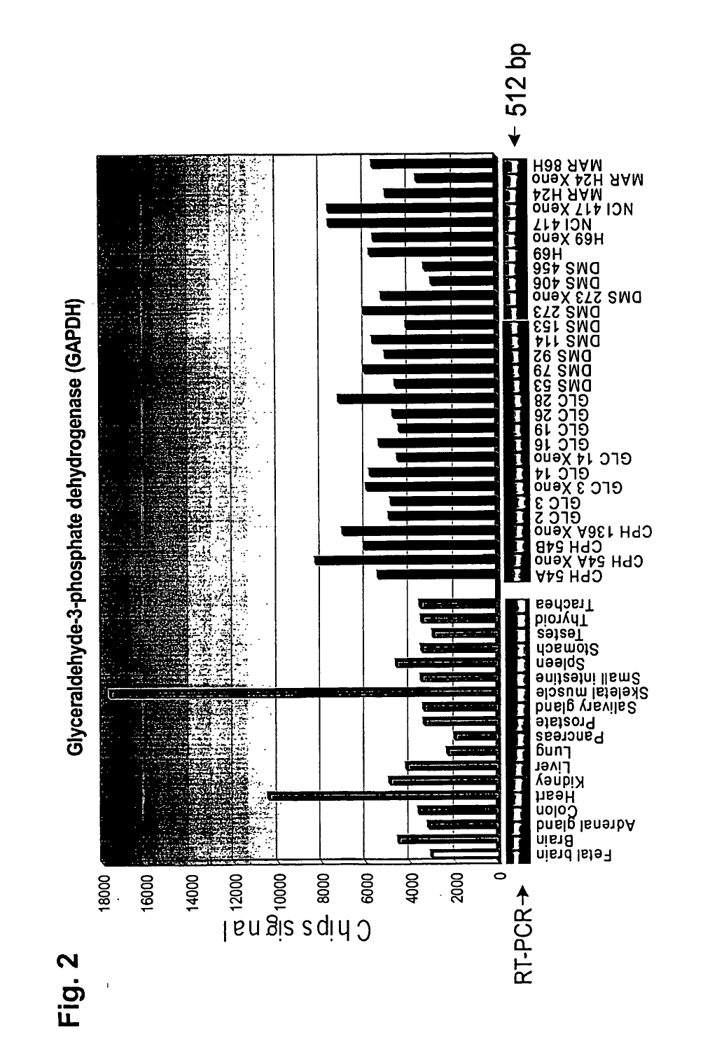

Clustering of the X-axis (samples) (FIG. 2) showed as expected that the SCLC cell lines clustered together, with Mar86MI and CPH54A being furthest apart. CPH54A and B clustered very close (B is a clonal variant of A), as did GLC14, GLC16 and GLC19 (derived from the same patient). Of the normal tissues expression from lung RNA obtained from Ambion and lung RNA obtained from CLONTECH clustered very close, as did 2 different batches of brain RNA obtained from CLONTECH with brain RNA obtained from Ambion. Expression from RNA obtained from fetal brain likewise clustered dose to the mature brain and was the closest of all normal tissues to the SCLC cell lines. This confirms that SCLC lines are of neuro-endocrinal origin. Clustering of the Y-axis (genes) clearly found 4 very distinct clusters of genes with higher expression in the SCLC cell lines. The smallest contained 19 genes, the second and third 65 each, and the fourth and largest gene cluster 268.

Selection Criterias for Candidate Promoters (First Nucleic Acid Sequences).

The candidate promoters were chosen based on expression level of the gene, which the promoter controls. The selection was performed on all 21 SCLC cell lines, but not xenografts and on 7 normal tissues (brain, adrenal gland, lung, kidney, heart, prostate, pancreas).

Selection was based on several criteria. Only genes, which scored present (P) in the absolute call and with an Average difference >50 (level of expression) were included. These output data were further processed in Microsoft Excel 2000. Genes were selected which were scored present in at least 11 of the 21 SCLC lines and if the gene was scored present in one or more normal tissues, the median Average difference value of the SCLC cell lines must be 4 times or more above the median Average difference value of the normal tissue. After a second screening using RNA from more normal tissues, the selected candidates are submitted to the same criteria as above and discarded if they do not fulfil the above requirements.

Validation of Chips Analysis by RT-PCR

Selected genes were analysed by semi-quantitative RT-PCR for verification of expression identified by Chips analysis. The quality of the cDNA was tested using primers for GADPH (Glyceraldehyde-3-phosphate dehydrogenase) (FIG. 2). All cDNA samples were very positive showing that the quality of the cDNA for further analysis was good.

RT-PCR with primers for Pro 221 (IA-1, insulinoma associated antigen 1) (FIG. 3) showed that in normal tissue adrenal gland and brain and fetal brain are weakly positive in both Chips and RT-PCR analysis. 4 SCLC lines or xenografts are negative in both analyses. All others are weak to very strongly positive. The RT-PCR and Chips analysis correlate extremely well.

RT-PCR with primers for Pro 30 (KIA0042) (FIG. 4) showed that in normal tissue testes is positive for both Chips and RT-PCR. Other normal tissues are low or negative by both analysis methods. All SCLC cells and xenografts are positive in both Chips and RT-PCR analysis. There are a few samples where the relative amounts in Chips and RT-PCR do not correlate (e.g. high in one and low in the other analysis).

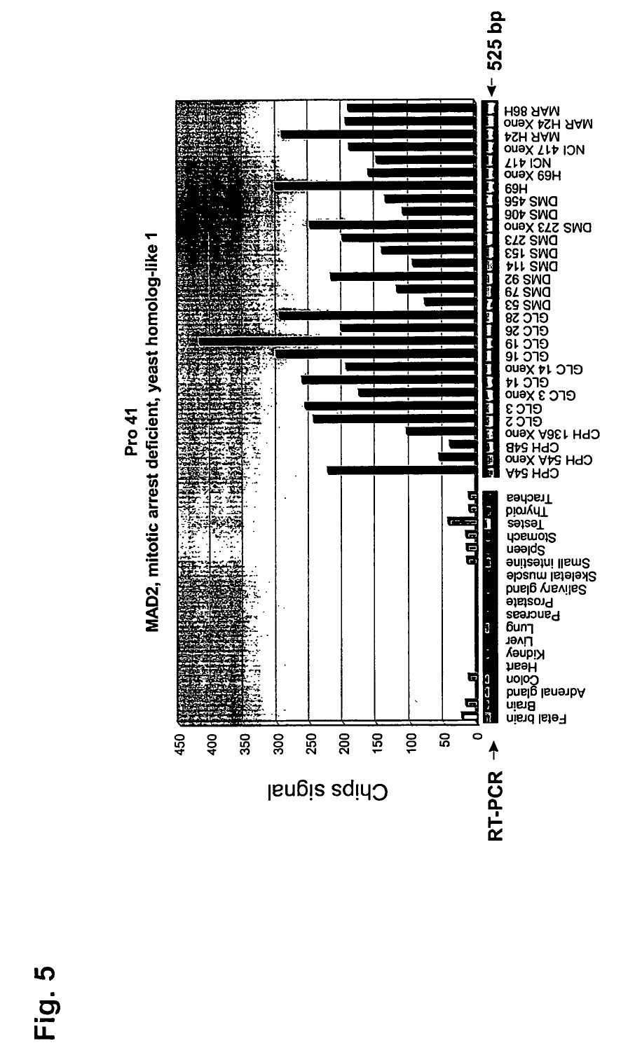

RT-PCR with primers for Pro 41 (MAD2) (FIG. 5) shows low expression in most normal tissues and high expression in testes measured both by Chips analysis and RT-PCR. All SCLC cell lines and xenografts show very high expression by both Chips and RT-PCR analysis.

RT-PCR with primers for Pro 210 (lamin B1) (FIG. 6) showed very low or no expression in normal tissues (colon positive for both assays). All SCLC and xenografts have high expression by Chips analysis—all except 2 are very positive by RT-PCR. RT-PCR values are arbitrarily chosen to match Chips signal.

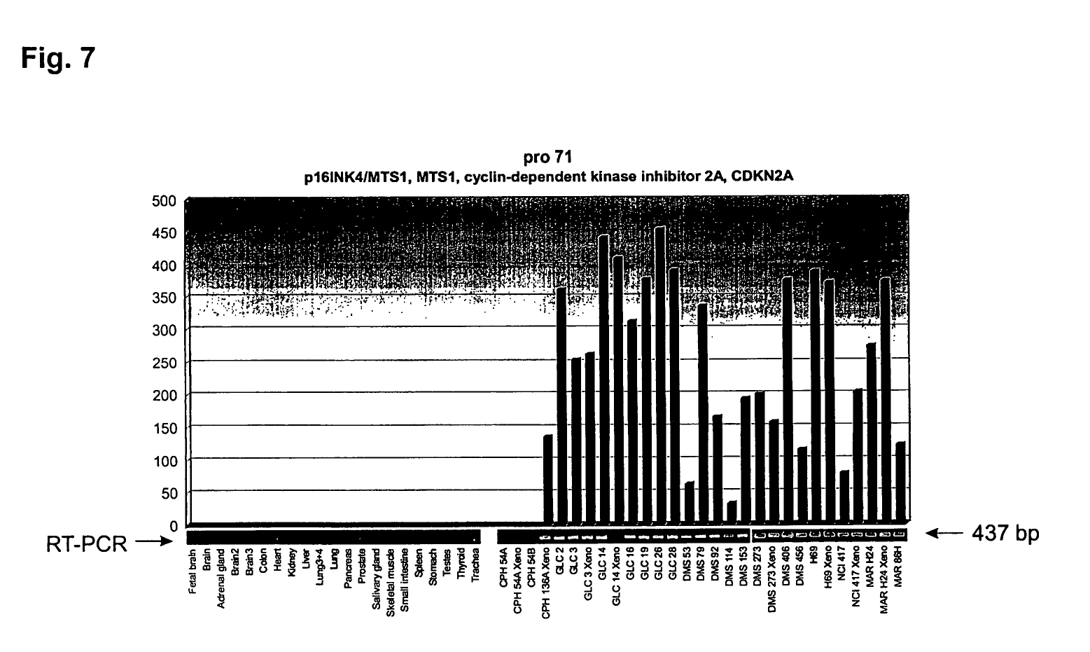

RT-PCR with primers for Pro 71 (CDKN2A) (FIG. 7) showed very low or no expression in normal tissues and high expression in all but 4 SCLC. Excepto for one sample negative in RT-PCR and positive in Chips analysis, the RT-PCR and Chips data correlate very well.

Conclusion on Validation of Chips Analysis by RT-PCR.

The Chips data and RT-PCR data correlate extremely well. The low to none expression in most normal tissues observed by Chips analysis is confirmed by the semi-quantitative RT-PCR reaction. The expression of the selected genes in SCLC cell lines and xenografts are very high and in all or most cell lines. Therefore using Chips analysis for identification of promoters with high and specific expression is an applicable method.

Selection Criteria's for Candidate Cell Surface Molecules Identified by Chips Analysis.

The first generation of candidate cell surface molecules were selected on basis of several criteria. The selection was performed on all 21 SCLC cell lines, but not xenografts and on 7 normal tissues (brain, adrenal gland, lung, kidney, heart, prostate, pancreas). Only genes, which scored present (P) in the absolute call and with an Average difference >50 were included. These output data were further processed in Microsoft Excel 2000. A gene was set to score one point for each cell line or tissue. The total scores for each gene were summarised for normal tissue and the SCLC cell lines, respectively. Genes were selected which were scored present in at least 5 of the 21 SCLC lines. A search was performed among these and candidate genes selected if one of the following words is included in the gene name: “receptor, membrane, adhesion, integrin, surface, antigen, syndecan, transport, channel, hormone, binding, glycoprotein, matrix, CAM, desmosome, gap junction, delta, immunoglobulin, MHC, CD, (HSPG, CSPG, integral, notch)”. The functions and cellular localisations of the proteins were unravelled based on database searches (NCBI: Nucleotide, Protein, Nucleotide, OMIM, PubMed, LocusLink). The best candidate genes were then selected based on these informations with emphasis on the function, cellular localisation and scores on expression (i.e. higher “expression score” for SCLC than for normal tissue). Furthermore, the expression in the different normal tissue is evaluated according to the specific tissues, in order to estimate the theoretical side effects. A second selection was performed on RNA from 21 SCLC cell lines, 8 of the cell lines grown as xenografts and 17 normal tissues (brain, lung, kidney, heart, trachea, adrenal gland, prostate, salivary gland, thyroid, liver, pancreas, spleen, small intestine, skeletal muscle, colon, stomach, testes) using the Affymetrix Microarray Suite™ version 5. Only expressed genes, which scored present (P) in the absolute call and with a signal >20 in at least 6 SCLC cell lines or xenografts were included. Further selection was performed as described above.

Validation of Chips Analysis by RT-PCR

Selected genes were analysed by semi-quantitative RT-PCR for verification of expression identified by Chips analysis. RT-PCR with primers for DR6 (TNFR related death receptor 6) (FIG. 8) shows medium expression in most normal tissues and medium to high in all except one SCLC line or xenograft. Chips analysis shows high expression in 2 normal tissues and high expression in 8 SCLC lines or xenografts. All positive by Chips analysis are also positive in RT-PCR.

RT-PCR % with primers for LRP8 (Apolipoprotein E receptor 2) (FIG. 9) shows low expression in 6 normal tissues and high expression in all SCLC lines and xenografts are positive by RT-PCR. All positives in Chips analysis are also positive in RT-PCR. RT-PCR values are arbitrarily chosen to match Chips signal.

RT-PCR with primers for NTPXR (neuronal pentraxin receptor) (FIG. 10) showed that all positive by Chips analysis are also positive by RT-PCR. There are more tissues and SCLC with positive expression as measured by RT-PCR, but expression is averagely higher in SCLC. Two SCLC samples are negative. RT-PCR values are arbitrarily chosen to match Chips signal.

RT-PCR with primers for NCAM1 (neural cell adhesion molecule) (FIG. 11) showed all samples positive by Chips analysis were also positive by RT-PCR. Several tissues and all except one SCLC are positive by RT-PCR only. One SCLC cell line is negative in both RT-PCR and Chips analysis. RT-PCR values are arbitrarily chosen to match Chips signal.

RT-PCR with primers for GluR2 (ionotropic glutamate receptor 2) (FIG. 12A) showed all samples positive by Chips analysis were also positive by RT-PCR. Both analysis showed very high expression in brain and RT-PCR low expression in adrenal gland. 4 SCLC cell lines are negative in both RT-PCR and Chips analysis. RT-PCR values are arbitrarily chosen to match Chips signal.

RT-PCR with primers for ITGAV (integrin alpha v subunit) (FIG. 12B). 5 samples are positive in Chips analysis, but negative by RT-PCR. Otherwise good correlation between Chips analysis and RT-PCR analysis. High expression in SCLC, but also in many tissues.

RT-PCR values are arbitrarily chosen to match Chips signal

Conclusion on Validation of Chips Analysis of Expression of Surface Molecules by RT-PCR.

Except for ITGAV all genes identified as expressed by Chips analysis were also found expressed when analysed by RT-PCR. More samples were positive when measured by RT-PCR. The expression of the selected genes in SCLC cell lines and xenografts are high and in many cell lines. Therefore using Chips analysis for identification of mRNA for surface molecules expressed by SCLC is an applicable method.

Validation of Chips Analysis by Western Blotting

Expression of selected gene products was analysed by western blotting using specific antibodies for comparison to of expression of mRNA identified by Chips analysis. Western blot analysis was only performed on SCLC cell lines and xenografts. Western blot analysis using antibodies to mGluR8 (metabotropic glutamate receptor 8) (FIG. 13) showed expression of mGluR8 protein in all SCLC cell lines and xenografts, whereas Chips analysis only detected expression in 8 samples. The intensities of the western blot do not correlate to the Chips values, but clearly show expression of mGluR8. Rat brain homogenate was used as positive control.

Western blot analysis using antibodies to NPTXR (neuronal pentraxin receptor) (FIG. 14) showed protein expression in all SCLC samples identified as having expression by Chips analysis. All samples are weak to strongly positive except GLC 28. DMS 153 has a prominent high molecular weight band also present in rat brain, which may be unprocessed or dimerised receptor. The protein amounts do not directly correlate with the Chips data, but clearly show expression in most SCLC. Rat brain homogenate was used as positive control.

Western blot analysis using antibodies to NCAM1 (neural cell adhesion molecule) (FIG. 15) showed expression of two isoforms of NCAM1 by all SCLC cell lines and xenografts except one, whereas Chips analysis identified expression in 14 samples. All samples positive by Chips analysis are positive by western blotting.

There is no obvious correlation between relative amounts in Chips analysis and western blotting.

Western blot analysis using antibodies to GluR2 (ionotropic glutamate receptor 2) (FIG. 16) showed expression in 9 samples. 6 samples were positive by Chips analysis, but negative by western blotting. However, the sensitivity of the antibody was not high. The other positive samples correlate well with the Chips analysis. Western blot analysis using antibodies to ITGAE (integrin alpha E subunit) (FIG. 17) showed expression in most SCLC samples. One sample was positive in Chips analysis and negative by Westren blotting. The relative intensities of expression between Chips analysis and western blotting do not correlate for many samples. A431 cell lysate was used as positive control.

Conclusion of western blot validation of Chips analysis on surface molecules For the selected surface molecules all genes identified as expressed by Chips analysis are also identified as expressed by western blotting showing that gene expression measured by Chips analysis is reflected in protein synthesis. For several genes western blotting identified expression in more samples than Chips analysis. Therefore the Chips analysis is an applicable method to identify surface molecules expressed by SCLC.

### Example 2

Surface Molecules Expressed by SCLC Cell Lines Identified by RT-PCR

Other expressed cell surface molecules were identified by the method of RT-PCR. mRNA was prepared from all 21 of the above listed cell lines using. Quick-Prep®mRNA Purification Kit (Pharmacia) according to manufacturers specifications. mRNA or total RNA from 29 different tissues was obtained from CLONTECH. The RNA was obtained from the following tissues: whole brain, spinal cord, small Intestine, kidney, heart, lung, testis, retina, bladder, stomach, uterus, liver, spleen, leukocyte, adipocyte, pituitary gland, ovary, mammary gland, prostate, trachea, thymus, adrenal gland, colon, pancreas, salivary gland, bone marrow, thyroid, lymph node and skeletal muscle.

Single-stranded cDNA synthesis was performed using the 1^{st} strand cDNA synthesis Kit for RT-PCR (Boehringer Mannheim) according to manufacturers instructions using an oligo-(dT)₁₅ primer.

Subsequent PCR with the cDNAs as template was performed in 10 mM Tris-Cl (pH 8.3), 50 mM KCl, 1 mM MgCl₂, 0.8 mM dNTPs, 0.4 μM primers and 0.12 U/μl Thermoprime plus DNA polymerase (Advanced Biotechnologies) with amplifications of 35 or 40 cycles of 95° C. for 30 sec, 62° C. for 30 sec and 72° C. for 1 min. A control reaction using GADPH primers was performed on all cDNAs. The PCR products 20 were analysed by agarose gel electrophoresis. Listed below are the gene products analysed, the sequence of primers with their position in the nucleotide sequence in the GenBank database and the percent of cell lines or tissues, which were positive for mRNA from the corresponding gene.

| | | | | % RT-PCR positive | |
|---|---|---|---|---|---|
| | GenBank | | | Normal | SLCL |
| Molecule | Acc. No. | Primer sequence | Position | tissue | lines |
| Atrial natriuretic | AF025998 | 5′- | 629-648 | 95.5% | 95.5% |
| peptide clearance | | AGCGGAACTGCTACTTC | | | |
| receptor | | ACC (SEQ ID 427) | | | |
| | | 5′- | 851-832 | | |
| | | TAGTCTCCACTGGTCAT | | | |
| | | GCC (SEQ ID 428) | | | |
| Gastrin/CCK-B | XM_006034 | 5′-GTGCGA- | 994-1015 | 100%^{#} | 100% |
| receptor | | ATGTTGCTGGTGATCG | | | |
| | | (SEQ ID 429) | | | |
| | | 5′-ACGGTGCATGAAGCAG | 1185-1164 | | |
| | | TAGACC (SEQ ID 430) | | | |
| Neuromedin B | M73482 | 5′AGATGGAAACACGGAA | 909-932 | 96.5% | 95.2% |
| receptor | | ACGCCTGG (SEQ ID 431) | | | |
| | | 5′-GGCTGTTGAA- | 1151-1128 | | |
| | | ATGCCTCCTGAAGC | | | |
| | | (SEQ ID 432) | | | |
| Glial cell line | NM_001496 | 5′- | 761-780 | 96.5% | 95.2% |
| derived neurotrophic | | TCTGCTTCTCCGACCCG | | | |
| factor α receptor | | CTT (SEQ ID 433) | | | |
| | | 5′-TAGCTGCA- | 1042-1023 | | |
| | | ATGGCCTCCGTG (SEQ ID 434) | | | |
| Bombesin receptor | XM_010317 | 5′- | 1289-1310 | 100%^{#} | 100% |
| (GRPR) | | CATGCTCCACTTTGTCA | | | |
| | | CCAGC (SEQ ID 435) | | | |
| | | 5′- | 1477-1456 | | |
| | | GAGGTCATGCAGGTTGT | | | |
| | | ACTCC (SEQ ID 436) | | | |
| Metabotropic glutamate | U92459 | 5′CCAGAGCTAAGTGATA | 598-621 | 21.1% | 95.2% |
| receptor 8 | | ACACCAGG (SEQ ID 437) | | | |
| | | 5′- | 801-825 | | |
| | | TTCACGTGGGATTTTCT | | | |
| | | GTGACTG (SEQ ID 438) | | | |

^{#}analysed on RNA from 7 normal tissues

The data from the RT-PCR experiments clearly suggest, that the metabotropic glutamate receptor 8 is a candidate receptor, as it is expressed in 95.2% of the SCLC cell lines, but only in 21.1% of normal tissues. Other receptors are also candidates, as they are expressed in more than 95% of the cell lines. A quantification of the relative levels of the RNA expression by real-time RT-PCR or northern blotting will further identify the suitable receptors.

### Example 3

Surface Molecules expressed by SCLC Cell Lines Identified by Western Blotting

18 of 19 tested SCLC lines from the same panel as above were found to express the surface molecules: the neural cell adhesion molecule (NCAM1) and cadherin (Rygaard et al., 1992). The expression in the SCLC cell lines was determined by western blotting utilizing polyclonal antibodies on protein extracts from the cell lines propagated both in vitro and as xenografts in nude mice. NCAM1 was detected by immunohistochemical methods in 20 of 20 surgically ressected SLCL tumours demonstrating that SCLC cells express NCAM1 in vivo (Kibbelaar, et al., 1991). NCAM1 is widely expressed during embryonic development, but is highly down regulated in the adult (reviewed in Gegelashvili and Bock, 1996), and therefore expressed at low levels in normal tissues from SCLC patients. It has already been demonstrated that NCAM1 expression is in part regulated by endocytosis (Minana et al., 2001) and that NCAM1 can be induced to internalise by antibody binding (Michalides et al., 1994). Cadherins has also been found to be endocytosed under normal (Kamei et al., 1999; Le et al., 1999) and pathological conditions (reviewed in Parkes and Hart, 2000). Therefore, these molecules are potential candidates for surface receptors for gene transfer.

### Example 4

Surface Molecules expressed by SCLC Cell Lines Identified by Other Methods

Several other cell surface receptors have been shown to be expressed by many SCLC cell lines and therefore are also potential candidates for surface receptors for gene transfer.

The expression of high affinity transforming growth factor-beta receptors. (TGF-β R) was demonstrated in several of the cell lines from the above panel by chemical crosslinking (Damstrup et al., 1993) By Northern blot analysis, the presence of the mRNA for TGF-β RI was found in 9 of 9 SCLC, TGF-β RII in 6 of 9 SCLC lines and of TGF-β RIII (betaglycan) in 9 of 9 SCLC lines (Nørgaard et al., 1996). Binding of the ligand to these receptors induce internalisation of the receptor (Anders et al., 1997, Dore et al., 2001).

The presence of insulin-like growth factor receptors (IGF-R) mRNA has been determined by RT-PCR and was found present in 14 of 14 examined SCLC lines (Quinn et al., 1996). The presence of both IGF-RL (Rotsch et al., 1992) and IGF-RII (Schardt et al., 1993) in 11 of 11 SCLC lines was demonstrated by Northern blotting, competitive binding assays and chemical crosslinking. Both receptors are known to internalise after ligand binding (Dore et al., 1997)

The epidermal growth factor receptor (EGF-R) and various homologues, variations or mutants (v-erb-B. HER2/neu (c-erb-2), ErbB3 and ErbB4 and EGF-R vIII) have been found expressed on a large number of cancer cell lines and tumours and several forms internalise after ligand binding (reviewed in Wells, 19990; Huang and Harari, 1999). By Northern blot analysis 11 of the 21 SCLC lines in the above panel were found to express EGF-R. The expression was verified by radioreceptor and affinity labelling analysis in 10 of the cell lines (Damstrup et al., 1992). Indeed, the EGF-R has been demonstrated to mediate targeted gene delivery in several of these SCLC lines (Cristano and Roth, 1996, Frederiksen et al., 2000).

### Example 5

Comparisons of Gene Expression of SCLC Cell Lines with Gene Expression of Additional Types of Normal Human Tissues and Other Tumour Cell Lines.

In order to further compare gene expression profiles between SCLC cell lines and normal tissues, total RNA from normal tissues from leukocyte will be obtained from, commercial sources (CLONTECH, Stratagene, Ambion or ResGen). Biotin labelled CRNA will be prepared as described above.

It is of importance to determine if the genes highly expressed in SCLC cell compared to normal tissues is a phenomena general for cancer cells or is SCLC specific. Therefore total RNA will be isolated from cell lines from other types of human cancers (e.g. commercially available cell lines derived from breast carcinoma, glioma, non small cell lung cancer (NCLC), colon carcinoma, neuroblastoma) and analysed as described above.

Comparison of Gene Expression of SCLC Cell Lines In Vitro with Gene Expression from SCLC Cells In Vivo.

Analysis of Gene Expression of SCLC Lines Propagated In Vivo

Selected cell lines are propagated in vivo as xenografts in both flanks of BALB/c nude mice according to Rygaard et al., 1992. When one of the tumours has reached the size of approximately 1 cm×1 cm the mice will be sacrificed and tumours removed. For total RNA isolation from the tumour, the tumour will be stored for 2448 hours in RNAlater™ (Ambion) and subsequently removed from the storage solution and stored at −70° C. until RNA preparation. Total RNA will be prepared from the tumours by extraction with Trizol (Life Technologies) according to manufacturers specifications. The total RNA will be further purified on RNeasy columns (Qiagen) according to the manufacturers method for RNA cleanup. Analysis of isolated total RNA and preparation of cDNA and biotin labelled cRNA and analysis of gene expression by Affymetrix Chips will be performed as described above. Protein extracts for Western blot analysis are prepared from freshly removed tumours by homogenisation on ice with a teflon pestel in 5 volumes (w/v) of 20 mM Tris-Cl (pH 7.5), 2% Triton X-100 with addition of protease and phophatase inhibitors (Protease Inhibitor Cocktail Set III and Phosphatase Inhibitor Cocktail Set II from Calbiochem) and subsequent clearing by high speed centrifugation (13.000×g).

Analysis of Gene Expression of Biopsies from Patients with Small Cell Lung Cancer

Biopsies from patients with diagnosed small cell lung cancer (obtained from Herlev Hospital) will be stored for 24-72 hours in RNAlater™ (Ambion) and subsequently removed from the storage solution and stored at −70° C. The tumours will be micro dissected by an experienced pathologist and RNA isolated from the tumours as above. RNA from several tumours will be pooled. Should the total RNA amount obtained not be sufficient for direct preparation of biotin labelled cDNA, the labelling procedure will be modified to include 2 further amplification steps as described In Ohyama et al., 2000.

### Example 6

Experimental Procedures for Identification of Cell Surface Molecules

Candidate cell surface molecules (receptors) expressed by SCLC cells are identified by Gene Chip analysis, Northern blotting, RT-PCR or by Western blotting. The specific splice form(s) expressed by the SCLC cells will be determined by RT-PCR and/or by sequencing (performed at GATC Biotech AG, Germany). The protein expression and subcellular localization of molecules, which are identified only on mRNA level, must be verified by other methods. If commercially antibodies are available, identification by western blotting (using protein extracts prepared from SCLC cell lines from the above panel propagated in vitro and in vivo as described above) and immunostaining of SCLC cell lines will be performed using the manufacturers recommendations.

For molecules with known ligands, which are commercially available or can be produced recombinantly (see below), this can additionally or alternatively be accomplished by binding or crosslinking studies. The labelled ligands (e.g. radio-, biotin- or fluorescent labelled ligands) will also be used to determine the affinity of the receptor, number of receptor molecules per cell and their ability for internalisation of the ligand.

For cell surface-molecules without known ligands, both the expression of the surface molecule and identification of ligands must be determined. As the mRNA encoding the cell surface molecule is readily available from the SCLC lines, the cDNA encoding the extracellular part can be cloned by standard RT-PCR methods into an expression vector to allow expression of a recombinant protein to be used for immunization. Preferably expression in a bacterial system (e.g. Qiagen pQE vectors) as a fusion with a suitable tag (e.g. 6×HIS) for easy purification of the recombinant protein will be used. Immunization for generation of polyclonal antibodies in rabbits will be performed at the Department of Experimental Medicine, The Panum Institute, University of Copenhagen. Generation of mouse monoclonal hybridomas will be performed at the Serum Institute, Copenhagen. Sera from immunized animals and conditioned medium from hybridomas will be screened for antigen binding using the recombinantly produced protein as immobilized antigen (in microtiter wells or on membranes). In addition, the specificity of the antibodies on the surface molecule, when expressed by mammalian cells, must be performed. This will be achieved by cloning the cDNA encoding the full length molecule into an eukaryotic expression vector (e.g. pcDNA 3.1 from Invitrogen or pCMV-Tag from CLONETCH) using RT-PCR. After transient transfection of a cell line, which does not endogenously express the molecule, the specificity of the antibodies will be determined using indirect immunofluorescence staining.

When a suitable antibody or serum has been identified, the protein expression will be analysed by immunostaining on the SCLC cell lines grown in vitro and in vivo and additionally on SCLC biopsies to verify of expression both in vitro and in vivo. The expression in normal tissues will be evaluated using a human tissue array containing 200 distinct tissue samples spotted on glass microscope slides (VastArray™ from GenRes).

Alternatively, human single chain antibodies isolated from a phage display library can be utilized (see below).

### Example 7

Experimental Procedures for Identification of Ligands to a Cell Surface Molecule and Determination of Their capacity for Internalisation

Known ligands, which are commercially available, will, when possible, be obtained in either a radio-, biotin- or fluorescent labelled form. For analyses of integrins as candidate surface molecules, the specific integrin alpha and beta subunit combination found in the cell lines must first be determined to identify the extracellular matrix ligand. This can be performed by immunostaining, as many antibodies against specific integrin combinations are commercially available.

If the ligand is commercially available, but not in a labelled form, the ligand can be labelled with ¹²⁵I (e.g. using the chloramine-T method) or with a fluorescent dye or biotin (e.g. using FluoReporter Kits from Molecular Probes). Binding assays will be performed to determine the specificity and capacity of ligand binding to the surface molecule. Using the labelled ligand, the ability of the surface molecule to internalise at 37° C. (with incubation at 0-4° C. as control) can be monitored after stripping of externally bound ligand (e.g. by acid or protease treatment) and measurement of internalised radioactivity for radio-labelled ligand; staining with enzyme or fluorescent labelled streptavidine for biotin labelled ligand or direct evaluation for fluorescent labelled ligand by microscopy.

If the ligand is known, but not commercially available, the gene encoding the ligand will be cloned into an expression vector using RT-PCR or obtaining a cDNA library from a suitable tissue or cell line or (when available) obtain the clone from commercial sources (GeneStorm® clones from Invitrogen or GeneConnection™ from CLONTECH). A suitable tag (e.g. 6×HIS) should be included in the recombinant ligand for easy purification. A bacterial expression system will be preferred. Recombinant expression will also enable the possibility to express the ligand as a fusion with EGFP for facilitating the analysis of binding and internalisation. Alternatively, antibodies against the tag can be used for analysis of binding and internalisation However, should posttranslational modifications such as glycosylation or sulfatation be essential for binding of the ligand to its receptor, expression as a secreted protein can be achieved in a yeast system (Pichia pastorius), in a insect system (Baculovirus) or in mammalian cells (e.g. HEK293, COS-7 or CHO cells).

If the ligand of a cell surface molecule is unknown, homology studies based on the genomic sequence or amino acid sequence of the receptor may result in identification of a superfamily of receptors to which the particular receptor belongs. A panel of ligands specific for this superfamily can then be tested using the methods described above. Alternatively, screening with a bacterial peptide expression library (e.g. FliTrx Random Peptide Display Library from Invitrogen) may identify of one or more peptide ligands. These peptide ligands can subsequently either be cloned for recombinant expression or obtained commercially. For this screening it would be optimal to use a cell line, which does not express the candidate surface molecule as, screening for non-specific binding and the same cell line transfected with an expression plasmid for the surface molecule for identification of specific peptide ligands.

If mouse monoclonal antibodies towards the cell surface molecule, have been generated, an alternative is to screen these antibodies for the capacity of internalising by detection of endocytosed antibodies by fluorescent labelled anti-mouse antibodies. Recombinantly expressed single chain antibodies cloned from the antibody producing hydbridoma will also be tested. For clinical trials, these antibodies must be humanized for example by the method described in Losman et al., 1999. If no internalising monoclonal antibodies are available, a phage library expressing human single chain antibody fragments can be used for isolation of internalising antibodies. By removing unspecific binding of phage displayed antibodies by incubation with a cell line negative for the cell surface molecule in question and selection with a transfected cell line expressing the molecule (as described above) specific and internalising antibodies can be identified and subsequently cloned from the phagemid DNA taken up by the cell after endocytosis (Nielsen and. Marks, 2000; Heitner et al., 2001) (collaboration with Prof. J, Engberg, Royal Danish School of Pharmacy).

### Example 8

Identification of a Promoter for Expression of a Therapeutic Gene.

The promoter region from genes, whose expression by GeneChips analysis has been found to be high in SCLC cell lines and xenografts and low or negative in normal tissues, are potential candidates to control and mediate expression of a therapeutic gene in targeted gene therapy. The expression by candidate promoters determined by GeneChips analysis will first be verified by RT-PCR or Northern blotting using several different primer sets or probes covering the entire molecule on the same RNAs used for GeneChips analysis (from SCLC cells and normal tissues) to ensure the cancer cell specificity of the promoter (as alternatively spliced variants expressed by the same promoter in normal tissues may not be recognized by the Affymetrix Chip). As the activity and specificity of a promoter can be encoded in a very large portion of DNA, it is essential to define the region(s) of the promoter, which are sufficient for specific and high expression in SCLC cells in order to limit the size of the DNA encoding the therapeutic gene to enhance delivery by a surface molecule. We set this limit to 15 kb, which is within the feasible size for cloning by PCR. Initially, a region of approx 15 kb upstream from the coding region of the candidate gene, including the region coding for the 5′ untranslated part of the mRNA, will be cloned by PCR using a thermostabile polymerase, which is capable of extending large PCR products with genomic DNA as template (e.g. Herculase from Stratagene). The primers used for PCR will be designed from the genomic sequence in the HUGO database and will be designed to contain either rare restriction sites for cloning by restriction cleavage or to contain loxP sites for direct cloning without restriction cleavage by addition of Cre recombinase. The vector to be used for testing the promoter regions will be constructed to contain a, promoterless gene encoding the Enhanced Green Fluorescent Protein (EGFP) from CLONTECH preceded by rare restriction sites in the multiple cloning sites (e.g. pd2EGFP-1 from CLONTECH) and/or a loxP site. The activity of the promoter will be estimated visually in a semi quantitative manner after transfection into the SCLC lines (e.g. using Lipfectamine Plus™ from Life Technologies) using fluorescence microscopy or quantitatively using a fluorometer (e.g. Victor 1420 from Wallac). As control for transfection efficiency, a low amount of plasmid encoding a red fluorescent protein under the control of a CMV promoter (pDsRed2-N1 from CLONTECH) will be used.

Promoters, which are active in the above assay, will be subcloned into smaller fragments (by PCR as described above or by standard restriction enzyme digestion) and tested for promoter activity as above. The relative activities of the promoters and subclones thereof can be determined quantitatively by recloning into a promoterless vector encoding a firefly luciferase and as transfection control, co-transfection with a plasmid encoding a renilla luciferase expressed from a SV40 promoter. Using the Dual-Luciferase® Reporter Assay System from Promega, the transcription from both plasmids in an extract of transiently transfected cells will be quantified using a luminometer (Lumat LB9507 from EG&G). In a similar manner, chimerics of the active parts of different, strong SCLC specific promoters can be tested for optimal expression and regulation. Alternatively, addition of enhancer sequences from other genes (e.g. viral enhancers) can be inserted. To ensure that the specificity of the selected promoter regions for SCLC cells compared to normal tissues is not lost in the various constructions, these will additionally be tested by transfection into commercially available cell lines of various origin derived from normal tissues. If higher specificity is needed, an additional specificity for cancer cells with mutations in p53 gene will be incorporated in the system. By inserting loxP sites adjacent to the promoter for the therapeutic gene and inserting the gene encoding Cre recombinase under the control of a p53 activated promoter, normal cells expressing wt p53 will express Cre recombinase which excises the promoter for the therapeutic gene, which therefore is not expressed.

If the transcriptional activity of the tumour specific promoter is not sufficient to achieve high enough levels of transcript encoding the therapeutic gene, it will be possible to utilize the specific promoter for activation of a second tissue-unspecific, but highly active promoter e.g. CMV. An example of this system is the encoding of Cre recombinase by the specific promoter, which after expression in the tumour tissue activates a CMV promoter by recombinational removal of a silencing element flanked by loxP sequences (Kijama et al., 1999).

In addition, the presence of endogenous transcriptional enhancers (e.g. steroid hormone receptor binding regions and receptors) will be determined. This will be analysed by transfection with the promoter controlling expression of EGFP or luciferase as described above, after addition of steroid hormones (e.g. retinoic acid, estrogen, progesteron or glucocorticoids). If present, these will give the opportunity to enhance the expression of the therapeutic gene by adjuvant administration of the hormone. Alternatively, these sequences can be inserted into the promoter for enhancement of transcriptional activity if the corresponding receptor is expressed by the SCLC cells.

### Example 9

Optimisation Methods for Complexing DNA with a Ligand

A complex formation between the DNA encoding the tissue specific promoter controlling expression of a therapeutic gene and the ligand must be achieved for specific internalisation. Several different possibilities will be tested. Biotin labelled ligand bound via streptavidine to biotin labelled poly-cationic poly-L-lysine (PLL) will complex with negatively charged DNA, thus forming a compacted ligand/DNA polyplex, which can be internalised via the ligand (Frederiksen et al., 2000). Biotinylation of ligand and poly-L-lysin of different sizes can be performed as described by Cristiano et al., 1996 or Wagner et al., 1990.

Alternatively, the commercially available branched cationic polymer polyethylenimine (PEI) can be used for forming the ligand/DNA complex. PEI/DNA complexes in themselves have a low activity of gene transfer. However, the activity and specificity can be substantially increased by covalent crosslinking of a ligand to PEI (Kircheis et al., 1997). Another possibility will be to test biotin labelled PEI combined with biotin labelled ligand and streptavidine, as described for PLL above.

A further advantage of this system over using PLL is that inclusion of an endosomal lysis agent in the complex is unnecessary (see below).

If the ligand is produced recombinantly, a different approach will also be tested. By including peptide sequences in the recombinant ligand, which can bind strongly to specific DNA sequences encoded in the DNA containing a therapeutic gene, it is possible to achieve a DNA/ligand complex, which then can be neutralized and compacted by PLL. The DNA binding domain from the yeast transcriptional activator GAL4 produced as a recombinant fusion with the ligand will be tested in this manner using DNA, where tandem repeats of the GAL4 recognition sequences have been incorporated into the DNA.

The above described complexes will initially be tested using DNA encoding EGFP controlled by a CMV promoter with a ligand known to bind a cell surface receptor capable of internalisation. The efficacy and specificity of will be determined by visual evaluation by fluorescence microscopy and/or by fluorometric quantification after administering to cells with and without expression of the receptor for the ligand.

### Example 10

Optimisation of Endosomal Lysis of Complex.

To avoid lysosomal degradation of the endocytosed complex, it is essential to include a endosomal lysis agent in the complex for release of the DNA into the cytoplasm or an agent such as Chloroquine, which raises the endosomal pH and thereby inhibits degradation by lysosomal enzymes (reviewed in Guy et al., 1995). Replication deficient adenovirus has been demonstrated as a potent endosmolytic agent, when directly coupled to the ligand/DNA polyplex (Yoshimura et al., 1993). However, the drawbacks of using deficient adenovirus or viral capsides is unwanted immunological response, unspecific uptake of the complex via viral receptors, safety precautions and difficulty in preparation and stability. Therefore, to avoid these disadvantages and in order to reduce the size of the complex, smaller, preferably non viral endosomolytic agents will be tested. The influenza virus hemagglutinin HA-2 N-terminal fusogenic peptides (Wagner et al., 1992), N-terminal rhino virus peptides, the pseudomonas exotoxin A translocation domain (Fominaya and Wels, 1996) and synthetic peptides (Gottschalk et al., 1996) have been found to mediate endosomal lysis or endosomal escape. Biotin labelled endosomolytic peptides can be included in the ligand/DNA complex, when generated by biotin labelled poly-L-lysine (PLL) coupled to streptavidin. Alternatively, when the ligand is produced recombinantly, the peptide sequences can be included in the N- or C-terminal part of the ligand. The efficiency of these peptides (added either separately or incorporated into a recombinant ligand) will be tested using DNA encoding a reporter gene (EGFP or luciferase) controlled by a CMV promoter complexed to a ligand known to internalise and the endosomal lysis monitored by evaluation of expression of the reporter gene. If the ligand/DNA complex is assembled by PEI, this agent can alone mediate endosomal swelling and subsequent lysis and release of the complex (Boussif et al., 1995).

### Example 11

Optimisation of Methods for Protection and Nuclear Targeting of the Therapeutic Gene.

To enhance the transport of endosomally released DNA encoding the therapeutic gene to the nucleus, the DNA will be covalently linked to a peptide encoding a nuclear targeting sequence (NLS—nuclear localization sequence). By excision of the therapeutic gene together with the promoter with restriction enzymes, protection of the DNA ends from digestion by exonucleases can be achieved by hybridisation to and ligation of oligonucleotides, which generate a protective stem-loop cap at the double stranded DNA ends. By including an amino-modified nucleotide in the oligonucleotide, this residue can be used for covalent crosslinking to a C-terminal amidated peptide encoding a nuclear localization signal (Zanta et al., 1999) (the peptides can be commercially obtained from e.g. Genosys, Tex., USA). A number of potential sequences are mentioned herein above. Initially, the enhancement of expression by coupling of a NLS peptide of simian virus 40 large tumour antigen to the DNA will be tested using a DNA fragment encoding EGFP with a CMV promoter and expression analysed by transient transfection of SCLC cell lines. Other peptides encoding NLS from other proteins (see herein above) will be tested for determination of the most efficient nuclear transport.

### Example 12

Experimental Procedures for Selection of Therapeutic Gene.

Potential therapeutic genes will be selected from the group of apoptosis inducing gene products, toxic gene products, gene products which introduce sensitivity towards harmless drugs, antisense RNA for oncogenes, Ribozymes targeted against oncogenes or genes encoding antibodies against oncogenes. The cDNA encoding the gene products for expression of protein or antisense RNA will either be obtained by cloning via RT-PCR, PCR on a cDNA library or obtained from commercial sources. To evaluate the efficacy of therapeutic genes for promoting cell death, these will inserted into a vector under the control of a CMV promoter and the effect of expression tested after transient transfection (e.g. using LipofectaminePlus, Life Technologies) into SCLC cell lines from the above panel, using a plasmid expressing EGFP for co-transfection for identification of transfected cells. For apoptosis inducing genes the effect of expression on transfected cells will be monitored by specific staining (e.g. by use of Vybrant Apoptosis Assay Kit from Molecular Probes). In addition, cell death of transfected cells will be monitored by the use of fluorescent ‘live stains’ (e.g. LIVE/DEAD Viability/Cytotoxicity Kit from Molecular Probes). Therapeutic genes selected from the experiments above will subsequently be recloned to be expressed under the control of one or more SCLC specific promoters and the efficiency of the expression analysed by transfection as described above.

### Example 13

Transduction Experiments In Vivo.

Once potential surface molecules and their ligands have been selected, a DNA/ligand complexing method including an endosomal lysis agent and nuclear targeting of a gene has been developed, the specificity and efficiency of the delivery system will be tested in vivo by administration of the complex to SCLC tumour xenografts of selected cell lines from the list above propagated in nude mice. A complex containing a reporter gene (e.g. β-galactosidase or EGFP) with a CMV promoter in an appropriate pharmaceutical formulation will be administered to the tumour xenografted mice by intravenous injection in the tail vein. After 24, 48′ or 72 hours, the mice will be sacrificed and the tumours and tissues from lung, liver, heart, brain, spleen, kidney and, skeletal muscle will be excised and stained or analysed for the product of the reporter gene (e.g. β-galactosidase).

Transduction Experiments Using Therapeutic Genes In Vivo.

To test the ability of the DNA/ligand to deliver a therapeutic gene in vivo, transduction experiments using therapeutic genes selected by in vitro experiments and DNA/ligand complexes selected from in vivo experiments will be performed as described above. If the therapeutic gene encodes a thymidine kinase, it will be accompanied by administration of a nucleotide analogue (e.g. gangcyclovir). Tumour development will be monitored by size determination, flow cytometry of cells from biopsies and after sacrifice of the mice, the tumours will be analysed for apoptosis and necrosis.

### REFERENCES

Anders R A, Arline S L, Dore J J, Leof E B.
Distinct endocytic responses of heteromeric and homomeric transforming growth factor beta receptors.
Mol Biol Cell. 1997 November;8(11):2133-43
Batt D G, Petraits J J, Houghton G C, Modi D P, Cain G A, Corjay M H, Mousa S A, Bouchard P J, Forsythe M S, Harlow P P, Barbera F A, Spitz S M, Wexler R R and Jadhav, P K (2000) Disubstituted Indazoles As Potent Antagonists of the Integrin Alpha(v)Beta(3). J Med Chem 43: pp 41-58.
:Bepler G, Jaques G, Neumann K, Aumuller G, Gropp C, Havemann K.
Establishment, growth properties, and morphological characteristics of permanent human small cell lung cancer cell lines.
J Cancer Res Clin Oncol. 1987;113(1):31-40.
Berendsen H H, de Leii L, de Vries E G, Mesander G, Mulder N H, de Jong B, Buys CH, Postmus P E, PopDema S, Sluiter H J, et al.
Characterization of three small cell lung cancer cell lines established from one patient during longitudinal follow-up.
Cancer Res. 1988 Dec. 1;48(23):6891-9.
Boger D L, Goldberg J, Silletti S, Kessler T and Cheresh D A (2001) Identification of a Novel Class of Small-Molecule Antangiogenic Agents Through the Screening of Combinatorial Libraries Which Function by Inhibiting the Binding and Localization of Proteinase MMP2 to Integrin Alpha(V)Beta(3). J Am Chem Soc 123: pp 1280-1288.
Brenner, M. B. and Cepek, K L Methods and compositions for modulating heterotypic Ecadherin interactions with T lymphocytes. [Patent Number: US6300080]. 2001.
Bruno V, Battaglia G, Copani A, D'Onofrio M, Di Iorio P, De Blasi A, Melchiorri D, Flor P J and Nicoletti F (2001) Metabotropic Glutamate Receptor Subtypes As Targets for Neuro-protective Drugs. J Cereb Blood Flow Metab 21: pp 1013-1033.
Bräuner-Osborne H. Egebjerg J, Nielsen E O, Madsen U and Krogsgaard-Larsen P (2000) Ligands for Glutamate Receptors: Design and Therapeutic Prospects. J Med Chem 43: pp 2609-2645.
Boussif O. Lezoualc'h F, Zanta M A, Mergny M D, Scherman D, Demeneix B, Behr J P.
A versatile vector for gene and oligonucleotide transfer into cells in culture and in vivo: polyethylenimine.
Proc Natl Acad Sci USA 1995 Aug. 1;92(16):7297-301
Brenner, M. B. and Cepek, K L. Methods and compositions for modulating heterotypic E-cadherin interactions with T lymphocytes. [Patent Number: US6300080]. 2001.
Bruno V, Battaglia G, Copani A, D'Onofrio M, Di Iorio P, De Blasi A, Melchiorri D, Flor P J and Nicoletti F (2001) Metabotropic Glutamate Receptor Subtypes As Targets for Neuroprotective Drugs. J Cereb Blood Flow Metab 21: pp 1013-1033.
Bräuner-Osborne H, Egebjerg J, Nielsen E O, Madsen U and Krogsgaard-Larsen P (2000) Ligands for Glutamate Receptors: Design and Therapeutic Prospects. J Med Chem 43: pp 2609-2645.
:Bulte J W, Go K G, Zuiderveen F, The TH, de Leji L.
Intracerebral and subcutaneous xenografts of human SCLC in the nude rat comparison of monoclonal antibody localization and tumor infiltrating lymphocytes.
J Neurooncol. 1993 April;16(1):11-8.
:Camey D N, Gazdar A F, BeDler G, Guccion J G, Maranoos P J, Moody T W, Zweig M H, Minna J D.
Establishment and identification of small cell lung cancer cell lines having classic and variant features.
Cancer Res. 1985 June;45(6):2913-23.
Cavalheiro E A and Olney J W (2001) Glutamate Antagonists: Deadly Liaisons With Cancer. Proc Natl Acad Sci USA 98: pp 5947-5948.
Chen C Y, Chang Y N, Rvan P, Linscott M, McGarritv G J, Chiang Y L.
Effect of herpes simplex virus thymidine kinase expression levels on ganciclovirmediated cytotoxicity and the “bystander effect”.
Hum Gene Ther. 1995 November;6(11):1467-76
:Chomczgnski P, Sacchi N.
Single-step method of RNA isolation by acid guanidinium thiocyanate-phenolchloroform extraction.
Anal Biochem. 1987 April,162(1):156-9.
Cristiano R J. Roth J A.
Epidermal growth factor mediated DNA delivery into lung cancer cells via the epidermal growth factor receptor.
Cancer Gene Ther. 1996 January-February;3(1):4-10
:Damstrup L, Ryaaard K, SDang-Thomsen M, Poulsen H S.
Expression of the epidermal growth factor receptor in human small cell lung cancer cell lines.
Cancer Res. 1992 Jun. 1;52(11):3089-93.
:Damstrup L, Ryaaard K, Spang-Thomsen M, Skovgaard Poulsen H.
Expression of transforming growth factor beta (TGF beta) receptors and expression of TGF beta 1, TGF beta 2 and TGF beta 3 in human small cell lung cancer cell lines.
Br J Cancer. 1993 May;67(5):1015-21.
:de Leji L, Postmus P E, Buys C H, Elema J D, Ramaekers F, PopDema S, Brouwer M, van der Veen A Y, Mesander G, The TH.
Characterization of three new variant type cell lines derived from small cell carcinoma of the lung.
Cancer Res. 1985 December;45(12 Pt 1):6024-33.
Dodds D C, Omeis I A, Cushman S J, Helms J A and Perin M S (1997) Neuronal Pentraxin Receptor, a Novel Putative Integral Membrane Pentraxin That Interacts With Neuronal Pentraxin 1 and 2 and Taipoxin—Associated Calcium-Binding Protein 49. J Biol Chem 272: pp 21488-21494.
Dore S, Kar S, Quirion R.
Presence and differential internalization of two distinct insulin-like growth factor receptors in rat hippocampal neurons.
Neuroscience. 1997 May;78(2):373-83
:Dore J J Jr, Yao D, Edens M, Garamszeai N, Sholl E L, Leof E B.
Mechanisms of Transforming Growth Factor-beta Receptor Endocytosis and Intracellular Sorting Differ between Fibroblasts and Epithelial Cells.
Mol Biol Cell. 2001 March;12(3):675-84.
:Engelholm S A, Spang-Thomsen M, Vindelov L L, Brunner N, Nielsen M H, Hirsch F, Nielsen A, Hansen H H.
Comparison of characteristics of human small cell carcinoma of the lung in patients, 15 in vitro and transplanted into nude mice.
Acta Pathol Microbiol Immunol Scand [A]. 1986 September;94(5):325-36.
Evans J P (2001) Fertilin Beta and Other ADAMs As Integrin Ligands: Insights into Cell Adhesion and Fertilization. Bioessays 23: pp 628-639.
Foley A G, Hartz B P, Gallagher H C, Ronn L C, Berezin V, Bock E and Regan C M (2000) A Synthetic Peptide Ligand of Neural Cell Adhesion Molecule (NCAM) Igl Domain Prevents NCAM Internalization and Disrupts Passive Avoidance Learning. J Neurochem:74: pp 2607-2613.
:Fominaya J. Wels W.
Target cell-specific DNA transfer mediated by a chimeric multidomain protein. Novel non-viral gene delivery system.
J. Biol. Chem. 1996 May 3;271(18):10560-8.
Frederiksen K S, Petri A. Abrahamsen N, Poulsen H S.
Gene therapy for lung cancer.
Lung Cancer. 1999 March;23(3):191-207. Review
Frederiksen K S, Abrahamsen N, Cristiano R J, Damstrup L, Poulsen H S.
Gene delivery by an epidermal growth factor/DNA polyplex to small cell lung cancer cell lines expressing low levels of epidermal growth factor receptor.
Cancer Gene Ther. 2000 February;7(2):262-8
Gegelashvill, G. and Bock, E. (1996),
Cell recognition molecules of the immunoglobulin superfamily in the nervous system., Biomembranes, vol 3, 33-75
Gottschalk S, Cristiano R J, Smith L C, Woo S L.
Folate receptor mediated DNA delivery into tumor cells: potosomal disruption results in enhanced gene expression.
Gene Ther. 1994 May,1 (3):185-91.
Gottschalk S, Sparrow J T, Hauer J, Mims M P, Leland F E, Woo S L, Smith L C.
A novel DNA-peptide complex for efficient gene transfer and expression in mammalian cells.
Gene Ther. 1996 May;3(5):48-57.
Goula D, Benoist C, Mantero S, Merlo G, Levi G, Demeneix B A.
Polyethylenimine-based intravenous delivery of transgenes to mouse lung.
Gene Ther. 1998 September;5(9):1291-5.
Gunji Y, Ochiai T, Shimada H, Matsubara H.
Gene therapy for cancer.
Surg Today. 2000;30(11):967-73. Review.
Guy J, Drabek D, Antoniou M.
Delivery of DNA Into mammalian cells by receptor-mediated endocytosis and gene therapy.
Mol Biotechnol. 1995 June;3(3):23748. Review.
Hartman G D and Duggan M E (2000) Alpha(v)Beta(3) Integrin Antagonists As Inhibitors of Bone Resorption. Expert Opin Investig Drugs 9: pp 1281-1291.
Heitner T, Moor A, Garrison J L, Marks C, Hasan T, Marks J D.
Selection of cell binding and internalizing epidermal growth factor receptor antibodies from a phage display library.
J Immunol Methods. 2001 Feb. 1;248(1-2):17-30.
Huana S M, Harari P M.
Epidermal growth factor receptor Inhibition in cancer therapy: biology, rationale and preliminary clinical results.
Invest New Drugs. 1999;17(3):259-69. Review
Huang T F (1998) What Have Snakes Taught Us About Integrins? Cell Mol Life Sci 54: pp 527-540.
Jane D E, Hoo K, Kamboj R, Deverill M, Bleakman D and Mandeizys A (1997) Synthesis of Willardiine and 6-Azawillardiine Analogs: Pharmacological Characterization on Cloned Homomeric Human AMPA and Kainate Receptor Subtypes. J Med Chem 40: pp 3645-3650.
Jane. D. AMPA/Kainate receptors. WWW.2001. http://www.bris.ac.uk/synaptic/info/pharmacology/AMPA.html
:Kamei T, Matozaki T, Sakisaka T, Kodama A, Yokovama S, Pena Y F, Nakano K Takaishi K, Takai Y.
Coendocytosis of cadherin and c-Met coupled to disruption of cell-cell adhesion in MDCK cells-regulation by Rho, Rac and Rab small G proteins.
Oncogene. 1999 Nov. 18;18(48):677-684.
Kang I C, Lee Y D and Kim D S (1999) A Novel Disintegrin Salmosin Inhibits Tumor Angiogenesis. Cancer Res 59: pp 3754-3760.
Karecla P I, Green S J, Bowden S J, Coadwell J and Kilshaw P J (1996) Identification of a Binding Site for Integrin AlphaEbeta7 in the N-Terminal Domain of E-Cadherin. J Biol Chem 271: pp 30909-30915.
Kaufmann, O., Georgi, T., and Dietel, M. Utility of 123C3 monoclonal antibody against CD56 (NCAM) for the diagnosis of small cell carcinomas on paraffin sections. Hum. Pathol. 28[12], 1373-1378.1997.
Kerr J S, Slee A M and Mousa S A (2000) Small Molecule Alpha(v) Integrin Antagonists: Novel Anticancer Agents. Expert Opin Investig Drugs 9: pp 1271-1279.
Kibbelaar, R. E., Moolenaar, K E., Michalides, R. J., Van Bodegom, P. C., Vanderschueren, R. G., Wagenaar, S. S., Dingemans, K P., Bitter-Suermann, D., Dalesio, O., van Zandwijk, N., and. Neural cell adhesion molecule expression, neuroendocrine differentiation and prognosis in lung carcinoma. Eur. J. Cancer 27[4], 431-435. 1991.
Kijima T, Osaki T, Nishino K, Kumagai T, Funakoshi T, Goto H, Tachibana I, Tanio Y, Kishimoto T.
Application of the Cre recombinase/loxP system further enhances antitumour effects in cell type-specific gene therapy against carcinoembryonic antigen-producing cancer.
Cancer Res. 1999 Oct. 1;59(19):4906-11
Kircheis R, Kichler A, Waliner G, Kursa M, Ogris M, Felzmann T, Buchbermer M, Waaner E.
Coupling of cell-binding ligands to polyethylenimine for targeted gene delivery.
Gene Ther. 1997 May;4(5):409-18
Kirkpatrick L L, Matzuk M M, Dodds D C and Petin M S (2000), Biochemical Interactions of the Neuronal Pentraxins. Neuronal Pentraxin (NP) Receptor Binds to Taipoxin and Taipoxin-Associated Calcium-Binding Protein 49 Via NP1 and NP2. J Biol Chem 276: pp-17786-17792.
Kiselyov W, Berezin V, Maar T E, Soroka V, Edvardsen K, Schousboe A and Bock E (1997) The First Immunoglobulin-Like Neural Cell Adhesion Molecule (NCAM) Domain Is Involved In Double-Reciprocal Interaction With the Second Immunoglobulin-Like NCAM Domain and in Heparin Binding. J Biol Chem 272: pp 10125-10134.
Krogsgaard-Larsen, P., Brehm, L., Johansen, J. S., Vinzents, P., Lauridsen, J., and Curtis, D. R. Synthesis and structure-activity studies on excitatory amino acids structurally related to ibotenic acid. J Med Chem. 28[5], 673-679. 1985.
:Le T L, Yap A S, Stow J L.
Recycling of E-cadherin: a potential mechanism for regulating cadherin dynamics.

J. Cell Biol. 1999 Jul. 12;146(1):219-32
:Losman M J, Qu Z, Krishnan I S, Wang J. Hansen H J, Goldenberg D M, Leung S O.
Generation and monitoring of cell lines producing humanized antibodies.
Clin Cancer Res. 1999 October;5(10 Suppl):3101s-3105s.
Madsen U, Stensbøl T B and Krogsgaard-Larsen P (2001) Inhibitors of AMPA and Kainate Receptors. Curr Med Chem 8: pp 1291-1301.
Mercer B, Markland F and Minkin C (1998) Contortrostabin, a Homodimeric Snake Venom Disintegrin, Is a Potent Inhibitor of Osteoclast Attachment J Bone Miner Res 13: pp 409-414.
Michalides R, Kwa B, Springall D, van Zandwijk N, Koopman J, Hilkens J and Mooi W (1994) NCAM and Lung Cancer. Int J Cancer Suppl 8: pp 34-37.

Minana R, Duran J M, Tomas M, Renau-Piqueras J and Guerri C (2001) Neural Cell Adhesion Molecule Is Endocytosed Via a Clathrin-Dependent Pathway. Eur J Neurosci 13: pp 749-756.
Montgomery A M, Becker J C, Siu C H, Lemmon V P, Cheresh D A, Pancook J D, Zhao X and Reisfeld R A (1996) Human Neural Cell Adhesion Molecule L1 and Rat Homologue NILE Are Ligands for Integrin Alpha v Beta 3. J Cell Biol 132: pp 475-485.
Mountain A.
Gene therapy: the first decade.
Trends Biotechnol. 2000 March;18(3):119-28. Review.
Nettelbeck D M, Jerome V, Muller R.
Gene therapy: designer promoters for tumour targeting.
Trends Genet 2000 April;16(4):174-81. Review
Nielsen L L, Maneval D C.
P53 tumor suppressor gene therapy for cancer.
Cancer Gene Ther. 1998 January-February;5(1):5263. Review
Nielsen U B, Marks J D.
Internalizing antibodies and targeted cancer therapy: direct selection from phage display libraries.
Pharm. Sci. Technol. Today. 2000 August;3(8):282-291.
:Norcaard P, Spang-Thomsen M, Poulsen H S.
Expression and autoregulation of transforming growth factor beta receptor mRNA In small-cell lung cancer cell lines.
Br J Cancer. 1996 May;73(9):1037-43.
:Ohyama H, Zhana X, Kohno Y, Alevizos I, Posner M, Wona D T, Todd R.
Laser capture microdissection-generated target sample for high-density oligonucleotide array hybridization.
Biotechniques. 2000 September;29(3):530-6.
:Parkes R J, Hart S L.
Adhesion molecules and gene transfer.
Adv Drug Deliv Rev. 2000 Nov. 15;44(2-3):135-62. Review
:Pettengill O S, Sorenson G D, Wurster-Hill D H, Curphey T J, Noll W W, Cate C C, Maurer L H.
Isolation and growth characteristics of continuous cell lines from small-cell carcinoma of the lung.
Cancer. 1980 Mar. 1;45(5):906-18.
Quinn K A, Treston A M, Unsworth E J, Miller M J, Vos M. Grimley C. Baftev J, Mulshine J L, Cutfitta F.
Insulin-like growth factor expression in human cancer cell lines.
J. Biol. Chem. 1996 May 10;271(19):11477-83.
Riddell D R, Vinogradov D V, Stannard A K, Chadwick N and Owen J S (1999) Identification and Characterization of LRP8 (ApoER2) in Human Blood Platelets. J Lipid Res 40: pp 1925-1930.
Roth J A, Ncauven D, Lawrence D D, Kemp B L, Carrasco C H, Ferson D Z, Hong W K, Komaki R, Lee J J, Nesbitt J C, Pisters K M, Putnam J B, Schea R. Shin D M, Walsh G L, Dolormente M M, Han C I, Martin F D, Yen N, Xu K, Stephens L C, McDonnell T J, Mukhopadhyay T, Cai D.
Retrovirus-mediated wild-type p53 gene transfer to tumors of patients with lung cancer.
Nat Med. 1996 September;2(9):985-91.
:Rotsch M, Maasbemg M, Erbil C, Jacues G, Worsch U, Havemann K.
Characterization of insulin-like growth factor I receptors and growth effects in human lung cancer cell lines.
J Cancer Res Clin Oncol. 1992; 118(7):502-8.
:Rygaard K, Moller C, Bock E, Spang-Thomsen M.
Expression of cadherin and NCAM in human small cell lung cancer cell lines and xenografts.
Br J Cancer. 1992 April;65(4):573-7.
Rønn L C, Olsen M, Ostergaard S, Kiselyov V, Berezin V, Mortensen M T, Lerche M H, Jensen P H. Soroka V, Saffell J L, Doherty P, Poulsen F M, Bock E, Holm A and Saffells J L (1999) Identification of a Neuritogenic Ligand of the Neural Cell Adhesion Molecule Using a Combinatorial Library of Synthetic Peptides. Nat Biotechnol 17: pp 1000-1005.
:Schardt C, Rotsch M, Erbil C, Goke R, Richter G, Havemann K.
Characterization of insulin-like growth factor II receptors in human small cell lung cancer cell lines.
Exp Cell Res. 1993 January;204(1):22-9.
Taraszka K S, Higgins J M, Tan K, Mandelbrot D A, Wang J H and Brenner M B (2000) Molecular Basis for Leukocyte Integrin Alpha(E)Beta(7) Adhesion to Epithelial (E)-Cadherin. J Exp Med 191: pp 1555-1567.
Thomas N K, Wright R A, Howson P A, Kingston A E, Schoepp D D and Jane D E (2001) (S)-3,4-DCPG, a Potent and Selective MGlu8a Receptor Agonist, Activates Metabotropic Glutamate Receptors on Primary Afferent Terminals in the Neonatal Rat Spinal Cord. Neuropharmacology 40: pp 311-318.
Turner J P and Salt T E (1999) Group III Metabotropic Glutamate Receptors Control Corticothalamic Synaptic Transmission in the Rat Thalamus in Vitro. J Physiol 519 Pt 2: pp 481-491.
Yoshimura K, Rosenfeld M A, Seth P, Crystal R G.
Adenovirus-mediated augmentation of cell transfection with unmodified plasmid vectors.
J. Biol. Chem. 1993 Feb. 5;268(4):2300-3
Wagner E, Zenke M, Cotten M, Beuq H, Bimsfiel M L,
Transferrin-polycation conjugates as carriers for DNA uptake into cells.
Proc Natl Acad Sci USA. 1990 May;87(9):3410-4
Wagner E, Plank C, Zatloukal K, Cotten M, Birnstiel M L.
Influenza virus hemagglutinin HA-2 N-terminal fusogenic peptides augment gene transfer by transferrin-polylysine-DNA complexes: toward a synthetic virus-like gene-transfer vehicle.
Proc Natl Acad Sci USA. 1992 Sep. 1;89(17):7934-8
Wells A.
EGF receptor.
Int J Biochem Cell Biol. 1999 June;31(6):63743. Review
Yoneda Y, Kawajiri S, Sugimura M, Osanai K, Kito F, Ota E and Mimura T (2001) Synthesis of Diaminobutane Derivatives As Potent Ca(2+)-Permeable AMPA Receptor Antagonists. Bioorg Med Chem Lett 11: pp 2663-2666.
Yu A, Choi J, Ohno K, Levin B, Rom W N and Meruelo D (2000) Specific Cell Targeting for Delivery of Toxins into Small-Cell Lung Cancer Using a Streptavidin Fusion Protein Complex. DNA Cell Biol 19: pp 383-388.
Zanta M A, Belauise-Valladier P, Behr J P.
Gene delivery a single nuclear localization signal peptide is sufficient to carry DNA to the cell nucleus.
Proc Natl Acad Sci USA. 1999 Jan. 5;96(1):91-6

1. A method for identifying a plurality of cell surface molecules, which are expressed at a different level in malignant cells compared with normal cells, comprising the steps of:
i) providing at least 3 malignant cell lines selected from the group consisting of CPH 54 A, CPH 54 B, GLC 2, GLC 3, GLC 14, GLC 16, GLC 19, GLC 26, GLC 28, DMS 53, DMS 79, DMS 92, DMS 114, DMS 153, DMS 273, DMS 406, DMS 456, NCI H69, NCI N417, MAR H24, MAR 86 MI, SHP-77, NCI-H2171, NCI-H2195, NCI-H2196, NCI-H2198, NCI-H2227, NCI-H2286, NCI-H2330, NCI-H735, NCI-H1339, NCI-H1963, NCI-H2107, NCI-H2108, NCI-H1304, NCI-H1341, NCI-H1417, NCI-H1436, NCI-H1522, NCI-H1618, NCI-H1672, NCI-H1694, NCI-H1836, NCI-H1870, NCI-H1876, NCI-H1882, NCI-H1926, NCI-H1930, NCI-H1994, NCI-H2029, NCI-H2059, NCI-H2066, NCI-H2081, NCI-H2141, NCI-H211, NCI-H220, NCI-H250, NCI-H524, NCI-H592, NCI-H711, NCI-H719, NCI-H740, NCI-H748, NCI-H774, NCI-H841, NCI-H847, NCI-H865, NCI-H1048, NCI-H1059, NCI-H1092, NCI-H1105, NCI-H1184, NCI-H1238, NCI-H1284, NCI-H1688, NCI-H187, NCI-H378, NCI-H526, NCI-H660, NCI-H889, NCI-H60, NCI-H196, NCI-H446, NCI-H209, NCI-H146, NCI-H82, NCI-H460, NCI-H345, NCI-H510A, NCI-128, NCI-446, SW 1271; and
ii) providing at least 3 total RNA samples derived from normal tissue selected from the group consisting of liver, heart, kidney, lung, adrenal gland, colon, pancreas, small intestine, spleen, skeletal muscle, trachea, prostate, placenta, salivary gland, testes, leucocytes, brain, adipose tissue, bladder, breast, cervix, esophagus, larynx, ovary, rectum, skin, spinal cord, stomach, thymus, thyroid and uterus; and
iii) comparing the expression of mRNA in the cell lines according to i) and tissue samples according to ii); and
iv) identifying nucleic acid sequences, wherein
a) there is a difference between the amount of mRNA expressed in one or more cell lines according to i) and the amount of mRNA expressed in one or more tissues according to ii); and/or
b) there is essentially no difference in the amount of mRNA expressed in at least two cell lines according to i); and/or
c) there is essentially no difference in the amount of mRNA expressed in at least two tissue samples according to ii); and
v) selecting among the nucleic acid sequences according to iv), nucleic acid sequences encoding for potential cell surface molecules.

2. (Canceled)

3. The method according to claim 1, wherein step ii) involves tissue samples derived from lung, liver, heart, and kidney.

4.-12. (Canceled)

13. The method according to claim 1, wherein nucleic acid sequences encoding for potential cell surface molecules according to step v) are selected according to information available in commonly accessible databases selected from the group consisting of PubMed (NCBI), Nucleotide (NCBI), Protein (NCBI), Structure (NCBI), OMIM (NCBI) and LocusLink (NCBI).

14.-16. (Canceled)

17. A method of identifying first nucleic acid sequences, which are capable of directing expression of second nucleic acid sequences operably linked thereto, wherein the level of said expression is different in malignant cells compared with normal cells comprising the steps of:
i) providing at least 3 malignant cell lines selected from the group consisting of CPH 54 A, CPH 54 B, GLC 2, GLC 3, GLC 14, GLC 16, GLC 19, GLC 26, GLC 28, DMS 53, DMS 79, DMS 92, DMS 114, DMS 153, DMS 273, DMS 406, DMS 456, NCI H69, NCI N417, MAR H24, MAR 86 MI, SHP-77, NCI-H2171, NCI-H2195, NCI-H2196, NCI-H2198, NCI-H2227, NCI-H2286, NCI-H2330, NCI-H735, NCI-H1339, NCI-H1963, NCI-H2107, NCI-H2108, NCI-H1304, NCI-H1341, NCI-H1417, NCI-H1436, NCI-H1522, NCI-H1618, NCI-H1672, NCI-H1694, NCI-H1836, NCI-H1870, NCI-H1876, NCI-H1882, NCI-H1926, NCI-H1930, NCI-H1994, NCI-H2029, NCI-H2059, NCI-H2066, NCI-H2081, NCI-H2141, NCI-H211, NCI-H220, NCI-H250, NCI-H524, NCI-H592, NCI-H711, NCI-H719, NCI-H740, NCI-H748, NCI-H774, NCI-H841, NCI-H847, NCI-H865, NCI-H1048, NCI-H1059, NCI-H1092, NCI-H1105, NCI-H1184, NCI-H1238, NCI-H1284, NCI-H1688, NCI-H187, NCI-H378, NCI-H526, NCI-H660, NCI-H889, NCI-H60, NCI-H196, NCI-H446, NCI-H209, NCI-H146, NCI-H82, NCI-H460, NCI-H345, NCI-H510A, NCI-128, NCI-446 and SW 1271, and
ii) providing at least 3 RNA samples derived from normal tissue samples derived from the group consisting of liver, heart, kidney, lung, adrenal gland, colon, pancreas, small intestine, spleen, skeletal muscle, trachea, prostate, placenta, salivary gland, testes, leucocytes, brain, adipose tissue, bladder, breast, cervix, esophagus, larynx, ovary, rectum, skin, spinal cord, stomach, thymus, thyroid and uterus; and
iii) comparing the expression of mRNA in the cell lines according to i) and tissue samples according to ii); and
iv) identifying second nucleic acid sequences, wherein
a) there is a difference between the amount of mRNA expressed in one or more cell lines according to i) and the amount of mRNA expressed in one or more tissues according to ii); and/or
b) there is essentially no difference in the amount of mRNA expressed in at least two cell lines according to i); and/or
c) there is essentially no difference in the amount of mRNA expressed in at least two tissue samples according to ii); and
v) identifying first nucleic acid sequences operably linked to the second nucleotide sequences identified in step iv)

18. (Canceled)

19. The method according to claim 17, wherein step ii) involves tissue samples derived from the group consisting of lung, liver, heart and kidney.

20.-30. (Canceled)

31. The method according to claim 17, wherein any first nucleic acid sequence operably linked to a second nucleic acid sequence comprises up to up to 5000 base pairs upstream of the translation start codon of said second nucleic acid sequence on the chromosome.

32. (Canceled)

33. The method according to claim claim 17, wherein any first nucleic acid sequence operably linked to a second nucleic acid sequence comprise intron sequences found downstream of the translation start codon of said second nucleic acid sequence on the chromosome.

34. The method according to claim 17, wherein any first nucleic acid sequence operably linked to a second nucleic acid sequence comprise an enhancer sequence located more than 10,000 base pairs upstream or downstream from the translation start codon of said second nucleic acid sequence on the chromosome.

35.-36. (Canceled)

37. A targeting complex comprising:
vi) a binding partner capable of binding a cell surface molecule identified by the method according to claim 1, wherein said cell surface molecule is selected from the group consisting of GRIA2, GRM8, ITGAV, ITGAE, NCAM1, NPTXR, LRP8 and CHRNA5; and
vii) a bioreactive species

38.-41. (Canceled)

42. The targeting complex according to claim 37, wherein the binding partner is selected from the group consisting of L-glutamate, kainate, 5-(bromomethyl)-4-isoxazolepropionic acid, analogues of glutamate, substituted quinoxaline 2,3 diones, GYKI52466, 5-I-Willardine, 5-F-Willardine, agonist and antagonist ligands to the AMPA ((RS)-αx-Amino-3-hydroxy-5-methyl-4-isoxazolepropionic acid, NBQX, CNQX, DNQX, GYKI 52466, 6-Chlorokynurenic acid, JSTX, L-APA, L-SOP, ACPT, (R,S)-PPG, CPPG, MAP4, (S)-3,4-DCPG, vitronectin, cytactin, fibronectin, fibrinogen, laminin, MMP-2, osteopontin, prothrombin, thrombospondin, von Willebrandts Factor, recombinant fragments of L1CAM, salmosin, E-cadherin and peptides thereof, including the peptide: NRDKETKV, NCAM1 domain Ig I+II, NCAM1 domain IgIII and peptides thereof, peptides C3: ASKKPKRNIKA, D3: AKKERQRKDTU, D4: ARALNWGAKP, monoclonal antibody 123C3, NPTX1, NPTX2, taipoxin, TCBP49, Oxynor, ApoE2, ApoE3, ApoE4, peptides from ApoE (E_{141;-155}; LRKLRKRLLRDADDL and its tandem E_{(141;-155)2}; LRKLRKRLLRDADDLLRKLRKRLL RDADDL) reelin, nicotine, acetylcholine, α-bungarotoxin, carbachol and specific antibodies against any of said surface molecules.

43. The targeting complex according to claim 37, wherein said cell surface molecule is capable of internalising the targeting complex.

44. The targeting complex according to claim 37, wherein said bioreactive species comprises a nucleic acid.

45. The targeting complex according to claim 44, wherein the nucleic acid comprises a second nucleic acid operably linked to a first nucleic acid sequence comprising an expression signal.

46. A targeting complex comprising
i) a binding partner capable of binding a cell surface molecule identified by the method according to claim 1, wherein said cell surface molecule is capable of internalising the targeting complex; and
ii) a bioreactive species comprising a nucleic acid sequence comprising a second nucleic acid operably linked to a first nucleic acid sequence comprising an expression signal, wherein said first nucleic acid sequence has been identified by the method according to claim 17.

47. The targeting complex according to claim 46, wherein the cell surface molecule comprises or essentially consists of a cell surface molecule selected from the group consisting of NCAM1, NPTXR, LRP8 and CHRNA5.

48. The targeting complex according to claim 46, wherein the cell surface molecule comprises or essentially consists of NCAM1.

49. The targeting complex according to claim 46, wherein the cell surface molecule comprises or essentially consists of NPTXR.

50.-51. (Canceled)

52. The targeting complex according to claim 46, wherein the binding partner is selected from the group consisting of NCAM1 domain Ig I+II, NCAM1 domain IgIII and peptides thereof, peptides C3: ASKKPKRNIKA, D3: AKKERQRKDTU, D4: ARALNWGAKP, monoclonal antibody 123C3, NPTX1, NPTX2, taipoxin, TCBP49, Oxynor, ApoE2, ApoE3, ApoE4, peptides from ApoE (E_{141;-155}; LRKLRKRLLRDADDL and its tandem E_{(141;-155)2}; LRKLRKRLLRDADDL-LRKLRKRLL RDADDL) reelin, nicotine, acetylcholine, α-bungarotoxin, carbachol and specific antibodies to said surface molecules.

53. The targeting complex according to claim 46, wherein said first nucleic acid sequence comprises an expression signal which direct a higher level of expression of said second nucleic acid sequence in malignant cells compared with non-malignant cells.

54. (Canceled)

55. The targeting complex according to claim 46, wherein said first nucleic acid sequence is selected from the group consisting of pro221, pro210, pro71, pro41, pro30, pro2, pro209, pro14, pro4, pro8, pro246, pro16, pro27, pro5, pro49, pro19, pro140, pro139, pro207, pro81, pro273 and pro362.

56. The targeting complex according to claim 46, wherein said first nucleic acid sequence comprises fragments of nucleotide sequences selected from the group consisting of pro221, pro210, pro71, pro41, pro30, pro2, pro209, pro14, pro4, pro8, pro246, pro16, pro27, pro5, pro49, pro19, pro140, pro139, pro207, pro81, pro273 and pro362.

57.-60. (Canceled)

61. The targeting complex according to claim 46, wherein said first nucleic acid sequence further comprises nucleic acid sequences not natively associated therewith.

62. (Canceled)

63. The targeting complex according to claim 46, wherein said second nucleic acid sequence encodes a therapeutic protein.

64.-69. (Canceled)

70. The targeting complex according to claim 37, wherein said bioreactive species is a toxin.

71. (Canceled)

72. The targeting complex according to claim 37, wherein said bioreactive species is an inducer of apoptosis.

73. (Canceled)

74. The targeting complex according to claim 37, wherein said bioreactive species comprises a radioisotope.

75. The targeting complex according to claim 37, wherein said bioreactive species comprises a cytostatica.

76. The targeting complex according to claim 37, wherein said bioreactive species comprises or essentially consists of a polypeptide.

77.-85. (Canceled)

86. The targeting complex according to claim 63, which comprises more than one first nucleotide sequence encoding a therapeutic protein or more than one therapeutic protein.

87.-89. (Canceled)

90. The targeting complex according to claim 37, wherein the complex further comprises a endosomal lytic agent selected from the group consisting of polyethylenimine (PEI), a replication defective virus and a viral protein capside.

91.-93. (Canceled)

94. The targeting complex according to claim 37, wherein the binding partner associates with the bioreactive species via a nucleic acid binding agent covalently attached to said binding partner.

95. The targeting complex according to claim 94, wherein the nucleic acid binding agent is selected from the group consisting of poly-L-lysine (PLL), spermine, spermidine and histone proteins.

96. (Canceled)

97. The targeting complex according to claim 37, wherein the binding partner associates with the bioreactive species indirectly via a pair of specific interacting components wherein one component is covalently attached to the bioreactive species and the second component is covalently attached to the binding partner.

98. (Canceled)

99. A method of using a cell surface molecule identified according to claim 1 as a drug target, wherein said drug target is capable of binding a binding partner and internalising said binding partner into cells expressing said cell surface molecule.

100. The method according to claim 99, wherein the cell surface molecule is selected from the group consisting of NCAM1, NPTXR, LRP8, CHRNA5, GRIA2, GRM8, ITGAV, ITGAE, TNFRSF12, L1CAM, GPR49 and TMEFF1.

101.-129. (Canceled)

130. A complex comprising a cell surface molecule identified according to claim 1 and a targeting complex comprising
i) a binding partner capable of binding said cell surface molecule, wherein said cell surface molecule is selected from the group consisting of GRIA2, GRM8, ITGAV, ITGAE, NCAM1, NPTXR, LRP8 and CHRNA5; and
ii) a bioreactive species.

131. The complex according to claim 130, wherein the cell surface molecule is selected from the group consisting of NCAM1, NPTXR, LRP8, CHRNA5, GRIA2, GRM8, ITGAV, ITGAE, TNFRSF12, L1CAM, GPR49, TMEFF.

132. (Canceled)

133. A pharmaceutical composition comprising of the targeting complex according to claim 37 together with a pharmaceutically acceptable carrier.

134. A method of treatment of a premalignant and/or malignant conditions in an individual in need thereof, comprising administering to said individual a pharmaceutically effective amount of the targeting complex according to claim 37.

135.-137. (Canceled)

138. The method according to claim 134, wherein said condition is a cancer selected from the group consisting of melanoma, brain tumour, neuroblastoma, breast cancer, lung cancer, prostate cancer, cervix cancer, uterine cancer, ovarian cancer, leukaemia, colon cancer, rectum cancer and bladder cancer.

139.-145. (Canceled)

146. The method according to claim 134, wherein said method further comprises one or more second treatments.

147.-159. (Canceled)

160. A method of using a pharmaceutically effective amount of a cell surface molecule identified according to claim 1, or a nucleic acid encoding said cell surface molecule, for the preparation of a vaccine.

161. (Canceled)

162. The method according to claim 160, wherein the cell surface molecule is selected from the group consisting of NCAM1, NPTXR, LRP8, CHRNA5, GRIA2, GRM8, ITGAV, ITGAE, TNFRSF12, L1CAM, GPR49 and TMEFF1.

163.-166. (Canceled)

167. The use method according to claim 160, wherein said vaccine is suitable for ameliorating and/or curative and/or prophylactic treatment of a premalignant and/or malignant conditions.

168. The targeting complex according to claim 45, wherein said second nucleic acid sequence encodes a therapeutic protein.
